(19) United States
(12) Patent Application Publication
(10) Pub. No.: US 2010/0041077 A1
(43) Pub. Date: Feb. 18, 2010

(54) PESTICIDE BIOMARKER

(76) Inventors: Jon Owen Nagy, Missoula, MT (US); Charles Mark Thompson, Missoula, MT (US)

Correspondence Address: Angelo Castellino, 5018 Merrimac Court, San Diego, CA, 92117, US

(21) Appl. No.: 12/514,797

(22) PCT Filed: Nov. 14, 2007
(86) PCT No.: PCT/US07/23954

§ 371 Date: May 13, 2009

Related U.S. Application Data
(60) Provisional application No. 60/858,849, filed on Nov. 13, 2006.

(51) Int. Cl. G01N 33/545; G01N 33/00; C12M 1/34; C40B 40/10; G01N 21/00

(52) U.S. Cl. 435/7.92; 435/287.2; 506/18; 436/531; 422/57

(57) ABSTRACT

Provided are methods, compositions and articles of manufacture for detecting biomarkers indicative of exposure of a mammal to organophosphate compounds. The interaction of such a biomarker with a receptor bound to a biopolymer results in an optical readout that reports the presence of the biomarker.

### CROSS-REFERENCE TO RELATED APPLICATIONS

This application is a national phase application under 35 USC §371 of international patent application no. PCT/US07/23954 filed Nov. 14, 2007, which claims priority from U.S. provisional application No. 60/858,849 filed on Nov. 13, 2006, both of which are incorporated by reference in their entireties.

### FIELD OF THE INVENTION

The invention relates to detection of a biomarker derived from interaction of a polypeptide with a compound or a metabolite thereof that covalently modifies the polypeptide. A particular embodiment disclosed is the detection of a biomarker resulting from an interaction of a serine hydrolase, such as acetylcholinesterase, with a organophosphoryl pesticide or a reactive organophosphoryl compound using an optical sensor incorporating an antibody that recognizes the biomarker and is immobilized onto a biopolymer material which undergoes a change in an optical property upon binding of the biomarker. Additionally disclosed are biosensor devices and optical sensor modules that are used or incorporated into these devices for biomarker detection.

### BACKGROUND OF THE INVENTION

The invention methods and devices for detection of a biomarker derived from interaction of a polypeptide with a compound or a metabolite thereof that covalently modifies the polypeptide. A particular embodiment disclosed is the detection of a biomarker resulting from an interaction of a serine hydrolase, such as acetylcholinesterase, with a organophosphoryl pesticide or a reactive organophosphoryl compound using an optical sensor incorporating an antibody that recognizes the biomarker and is immobilized onto a biopolymer material which undergoes a change in an optical property upon binding of the biomarker. Additionally disclosed are biosensor devices and optical sensor modules that are used or incorporated into these devices for biomarker detection.

In a particular embodiment of the invention a biomarker results from covalent modification of a serine hydrolase after interaction of the enzyme with a suicide inhibitor. In more particular embodiments the biomarker results from interaction of a serine hydrolase such as an acetylcholine esterase with an organophosphate compound or a metabolite thereof, which acts as the suicide inhibitor. The term “organophosphate” is used in the art to describe chemical classes of compounds comprising insecticides and pesticides. Such compounds are capable of modifying proteins and polypeptides, including cholinesterases, either directly or after activation by one or more metabolic processes. Organophosphoryl (OP) insecticides are organophosphate compounds, which include malathion, diazinon, chlorpyriphos and others (shown in Table 1), are the most widely used agrochemicals for the control of insect pests in the world and represents an exposure route for such environmental toxins to field workers. OP insecticides in structure 1a (see below) represents one class of organophosphoryl pesticides (organophoshothionate pesticide) having a P═S moiety in comparison to some examples of reactive organophosphoryl compounds represented by structures 1b and 2 which have a P═O moiety. Typically, an organophosphoryl pesticides requires metabolic processing, as shown in the conversion 1a (thionate) to 1b (P═O, oxon) to become active and toxic whereas reactive organophosphoryl compounds represented by structure (2) are in the oxon form and are directly toxic. The oxon forms (1b & 2) are primarily responsible for their neurotoxic mechanism of action (described further below).

Between 150,000 and 300,000 OP-related toxicity incidences are reported yearly in the US (Rosenstock, 1991) and several million people are

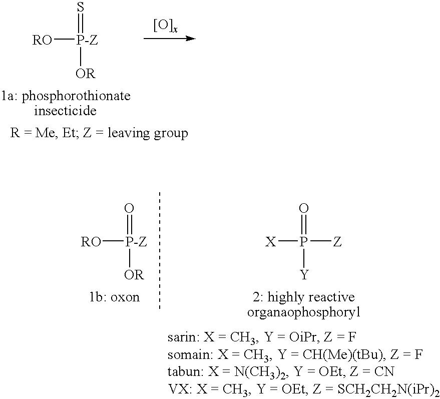

treated worldwide for exposure to OP insecticides. Owing to the mode of inhalation and toxic neurochemical mechanism of action, individuals at high risk to OP exposure include children and seniors, those with airway disabilities like asthma, neurological diseases, and/or mental illness. Subpopulations may be a greater risk owing to prior exposure to OP agents, such as farmers, agrochemical workers, applicators and other occupations that handle OP compounds. Between 1993 and 1996, about 65,000 cases of OP poisoning were reported to the US Poison Control Center and of these, 25,000 incidents involved children under age six. It is estimated that more than 1 million children age five and under (1 in 20) consume an unsafe dose of OPs in the US (Goldman, 2000).

The devices and methods described herein address particular needs relating to OP exposure, which includes monitoring for chronic or acute exposure from an organophosphate compound such as an organophosphoryl pesticide or a reactive organophosphoryl compound

The mechanism of OP action which is discussed for an acetylcholinesterase applies to other cholinesterases such as butyryl-cholinesterase and other proteins which provide biomarkers for OP exposure and is therefore not meant to be limiting of the inventions disclosed herein to a particular cholinesterase or protein. The key event in the mechanism of OP poisoning is the reaction of the OP compound with AChE to afford structurally unique products that represent mechanistically precise biomarkers of exposure. Henceforth, the term OP-AChE conjugates refers to the initially formed OP-conjugate from OP reaction with an acetylcholinesterase or a catalytically competent fragment thereof (a primary organophosphate biomarker) and subsequently formed aged derivatives (secondary organophosphate biomarkers) unless indicated otherwise. The present disclosure provides novel method to identify OP-AChE conjugates (i.e., organophosphate biomarkers) whose structures may be predicted from mechanistic considerations and represents an advance to the art.

Therefore, a need exists for detection of organophosphate biomarkers that is addressed by the present disclosure which provides for detection systems and methods for assessing the amounts, type and structure of a biomarker, such as an OP-AChE conjugate and its aged product, in order to assist with proper therapeutic intervention from exposure of a mammal to an environmental toxin such as an OP-compound and to evaluate threats from widespread dissemination of such toxins.

### SUMMARY OF THE INVENTION

Biosensor devices and optical sensor employed in such devices are described for detecting and discriminating bio-molecular products termed, in general, “biomarkers” resulting from exposure to a chemical compound. Specific biomarkers characteristic of organophosphate (OP) compound exposure are referred to as “OP-protein conjugates” or OP-polypeptide conjugates. The OP-protein or polypeptide conjugates are formed when an organophosphate (OP) compound such as an agricultural pesticide, including but not limited to malathion, diazinon and chlorpyriphos (and others shown in Table 1) modifies a polypeptide or protein such as an acetylcholinesterase (AChE). In those cases where the protein modified is an AChE, the resulting conjugates are referred to as “OP-AChE conjugates”. The biosensor devices and methods for detecting and quantifying exposure to an OP compound are novel since analysis is conducted for specific biomarkers that are represented by a distinct set of OP-protein conjugates. Therefore, the biosensor devices of the instant invention can identify individual OP-protein conjugates that together distinguish exposure to one OP compound from exposure to a different OP compound which presents a different set of OP-protein conjugates.

Novel biosensor devices employing receptor-modified PDA polymers which provide an efficient and rapid means of detecting OP-AChE conjugates that are biomarkers for exposure of an OP compound to an acetylcholinesterase or a catalytically competent fragment thereof are described. Receptors, including antibodies, Fab fragments, and other immunoglobulin fragment that contain a hyper-variable domain, and are capable of recognizing protein conjugates resulting from OP exposure to an acetylcholinesterase or a catalytically competent fragment thereof, are described and is one class of biomarker receptors. A biomarker receptor when immobilized to a biopolymer material such as PDA biopolymer films provides an optical sensor for detection of a biomarker. Also described are novel analytical methods using receptor-modified PDA polymers for monitoring the course of acute and chronic exposure to an OP compound. One embodiment of a biosensor device uses a fluorogenic, antibody-modified PDA polymer (an Ab-PDA biopolymer material). One embodiment of the invention that is described relates to exposure of acetylcholinesterase to an OP compound and applies to other embodiments involving other cholinesterases such as butyryl-cholinesterase and other proteins that provide biomarkers for OP exposure. Novel receptor-modified PDA polymers, which are comprised of specific recognition receptors such as antibodies that detect OP-AChE conjugates, are described. Also described are OP sensor modules which are comprised of receptor-modified PDA polymers. Further described is a biosensor device which uses one or more OP optical sensors or optical sensor modules for analyzing exposure to an OP compound and is useful for assessing the extent of exposure of a subject to an OP compound to provide useful information to guide therapeutic intervention.

### BRIEF DESCRIPTION OF THE DRAWINGS

FIG. 1A. An optical sensor having a biomarker receptor (e.g. anti OP-AchE antibody) immobilized onto a biopolymer material (e.g. a polydiacetylene polymer film)

FIGS. 1B and 1C. Example of a biomarker binding to a biomarker receptor immobilized onto a PDA-biopolymer film that induces a fluorescence change in the biopolymer material (250×250 μm views are captured using a standard rhodamine excitation and red LP emission filter set).

FIG. 2A. Flow chart for construction an OP-optical sensor module.

FIG. 2B. Diagram of a biosensor device.

### DETAILED DESCRIPTION

Definitions As used herein and unless otherwise stated or implied by context, terms that are used herein have the meanings defined below. Unless otherwise contraindicated or implied, e.g., by including mutually exclusive elements or options, in these definitions and throughout this specification, the terms “a” and “an” mean one or more and the term “or” means and/or.

At various locations in the present disclosure, e.g., in any disclosed embodiments or in the claims, reference is made to compounds, compositions, compositions, or methods that “comprise” one or more specified components, elements or steps. Invention embodiments also specifically include those compounds, compositions, compositions or methods that are or that consist of or that consist essentially of those specified components, elements or steps. The terms “comprising”, “consist of” and “consist essentially of” have their normally accepted meanings under U.S. patent law unless otherwise specifically stated. The term “comprised of” is used interchangeably with the term “comprising” and are stated as equivalent terms. For example, disclosed compositions, devices, articles of manufacture or methods that “comprise” a component or step are open and they include or read on those compositions or methods plus an additional component(s) or step(s). Similarly, disclosed compositions, devices, articles of manufacture or methods that “consist of” a component or step are closed and they would not include or read on those compositions or methods having appreciable amounts of an additional component(s) or an additional step(s).

“About” as used here in describing a numerical value or a range of a value means the numerical value or range is intended to encompass uncertainty in measurement of the value. The uncertainty will depend on the type of value to be measured and the method employed for determining the value. Such an uncertainty will be known or is readily determined by the skilled artisan by establishing the accuracy and precision of instrumentation and-or method used in determining the value.

“Alkyl” as used here means linked normal, secondary, tertiary or cyclic carbon atoms, i.e., linear, branched, cyclic or any combination thereof. Alkyl moieties, as used herein, may be saturated, or unsaturated, i.e., the moiety may comprise one, two, three or more independently selected double bonds or triple bonds. Unsaturated alkyl moieties include moieties as described below for alkenyl, alkynyl, cycloalkyl, and aryl moieties. Saturated alkyl groups contain saturated carbon atoms (sp³) and no aromatic, sp² or sp carbon atoms. The number of carbon atoms in an alkyl group or moiety can vary and typically is 1 to about 50, e.g., about 1-30 or about 1-20, unless otherwise specified, e.g., C₁₋₈ alkyl or C1-C8 alkyl means an alkyl moiety containing 1, 2, 3, 4, 5, 6, 7 or 8 carbon atoms and C₁₋₆ alkyl or C1-C6 means an alkyl moiety containing 1, 2, 3, 4, 5 or 6 carbon atoms.

When an alkyl group is specified, species may include methyl, ethyl, 1-propyl (n-propyl), 2-propyl (iso-propyl, —CH(CH₃)₂), 1-butyl (n-butyl), 2-methyl-1-propyl (iso-butyl, —CH₂CH(CH₃)₂), 2-butyl (sec-butyl, —CH(CH₃)CH₂CH₃), 2-methyl-2-propyl (t-butyl, —C(CH₃)₃), amyl, isoamyl, sec-amyl and other linear, cyclic and branch chain alkyl moieties. Unless otherwise specified, alkyl groups can contain species and groups described below for cycloalkyl, alkenyl, alkynyl groups, aryl groups, arylalkyl groups, alkylaryl groups and the like.

Cycloalkyl as used here is a monocyclic, bicyclic or tricyclic ring system composed of only carbon atoms. The number of carbon atoms in an cycloalkyl group or moiety can vary and typically is 3 to about 50, e.g., about 1-30 or about 1-20, unless otherwise specified, e.g., C₃₋₈ alkyl or C3-C8 alkyl means an cycloalkyl moiety containing 3, 4, 5, 6, 7 or 8 carbon atoms and C₃₋₆ alkyl or C3-C6 means an cycloalkyl moiety containing 3, 4, 5 or 6 carbon atoms. Cycloalkyl groups will typically have 3, 4, 5, 6, 7, 8, 9, 10, 11, 12, 13, 14, 15, 16, 17, 18, 19 or 20 carbon atoms and may contain exo or endo-cyclic double bonds or endo-cyclic triple bonds or a combination of both wherein the endo-cyclic double or triple bonds, or the combination of both, do not form a cyclic conjugated system of 4n+2 electrons; wherein the bicyclic ring system may share one (i.e., spiro ring system) or two carbon atoms and the tricyclic ring system may share a total of 2, 3 or 4 carbon atoms, typically 2 or 3.

When a cycloalkyl group is specified, species may include cyclopropyl, cyclopentyl, cyclohexyl, adamantly or other cyclic all carbon containing moieties. Unless otherwise specified, cycloalkyl groups can contain species and groups described for alkenyl, alkynyl groups, aryl groups, arylalkyl groups, alkylaryl groups and the like and can contain one or more other cycloalkyl moities. When cycloalkyl is used as a Markush group the cycloalkyl is attached to a Markush formula with which it is associated through an carbon involved in a cyclic carbon ring system carbon of the cycloalkyl group.

“Alkenyl” as used here means a moiety that comprises one or more double bonds (—CH═CH—), e.g., 1, 2, 3, 4, 5, 6 or more, typically 1, 2 or 3 and can include an aryl moiety such as benzene, and additionally comprises linked normal, secondary, tertiary or cyclic carbon atoms, i.e., linear, branched, cyclic or any combination thereof unless the alkenyl moiety is vinyl (—CH═CH₂). An alkenyl moiety with multiple double bonds may have the double bonds arranged contiguously (i.e. a 1,3 butadienyl moiety) or non-contiguously with one or more intervening saturated carbon atoms or a combination thereof, provided that a cyclic, contiguous arrangement of double bonds do not form a cyclically conjugated system of 4n+2 electrons (i.e., aromatic). The number of carbon atoms in an alkenyl group or moiety can vary and typically is 2 to about 50, e.g., about 2-30 or about 2-20, unless otherwise specified, e.g., C₂₋₈ alkenyl or C2-8 alkenyl means an alkenyl moiety containing 2, 3, 4, 5, 6, 7 or 8 carbon atoms and C₂₋₆ alkenyl or C2-6 alkenyl means an alkenyl moiety containing 2, 3, 4, 5 or 6 carbon atoms. Alkenyl groups will typically have 2, 3, 4, 5, 6, 7, 8, 9, 10, 11, 12, 13, 14, 15, 16, 17, 18, 18 or 20 carbon atoms.

When an alkenyl group is specified, species include, e.g., any of the alkyl or cycloalkyl moieties described above that has one or more double bonds, methylene (═CH₂), methylmethylene (═CH—CH₃), ethylmethylene (═CH—CH₂—CH₃), ═CH—CH₂—CH₂—CH₃, vinyl (—CH═CH₂), allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl and other linear, cyclic and branched chained all carbon containing moieties containing at least one double bond. When alkenyl is used as a Markush group the alkenyl is attached to a Markush formula with which it is associated through an unsaturated carbon of a double bond of the alkenyl group.

“Alkynyl” as used here means a moiety that comprises one or more triple bonds (—C≡C—), e.g., 1, 2, 3, 4, 5, 6 or more, typically 1 or 2 triple bonds, optionally comprising 1, 2, 3, 4, 5, 6 or more double bonds, with the remaining bonds (if present) being single bonds and comprising linked normal, secondary, tertiary or cyclic carbon atoms, i.e., linear, branched, cyclic or any combination thereof, unless the alkynyl moiety is ethynyl. The number of carbon atoms in an alkenyl group or moiety can vary and typically is 2 to about 50, e.g., about 2-30 or about 2-20, unless otherwise specified, e.g., C₂₋₈ alkynyl or C2-8 alkynyl means an alkynyl moiety containing 2, 3, 4, 5, 6, 7 or 8 carbon atoms. Alkynyl groups will typically have 2, 3, 4, 5, 6, 7, 8, 9, 10, 11, 12, 13, 14, 15, 16, 17, 18, 18 or 20 carbon atoms.

When an alkynyl group is specified, species include, e.g., any of the alkyl moieties described above that has one or more double bonds, ethynyl, propynyl, butynyl, iso-butynyl, 3-methyl-2-butynyl, 1-pentynyl, cyclopentynyl, 1-methyl-cyclopentynyl, 1-hexynyl, 3-hexynyl, cyclohexynyl and other linear, cyclic and branched chained all carbon containing moieties containing at least one triple bond. When an alkynyl substituent is used as a Markush group the alkynyl is attached to a Markush formula with which it is associated through an unsaturated carbon of a triple bond of the alkynyl group.

“Aryl” as used here means an aromatic ring system or a fused ring system with no ring heteroatoms comprising 1, 2, 3 or 4 to 6 rings, typically 1 to 3 rings; wherein the rings are composed of only carbon atoms; and refers to a cyclically conjugated system of 4n+2 electrons (Huckel rule), typically 6, 10 or 14 electrons some of which may additionally participate in exocyclic conjugation (cross-conjugated). When an aryl group is specified, species may include phenyl, naphthyl, phenanthryl and quinone. When aryl is used as a Markush group the aryl is attached to a Markush formula with which it is associated through an aromatic carbon of the aryl group.

“Alkylaryl” as used here means a moiety where an alkyl group is bonded to an aryl group, i.e., -alkyl-aryl, where alkyl and aryl groups are as described above, e.g., —CH₂—C₆H₅ or —CH₂CH(CH₃)—C₆H₅.

“Arylalkyl” as used here means a moiety where an aryl group is bonded to an alkyl group, i.e., -aryl-alkyl, where aryl and alkyl groups are as described above, e.g., —C₆H₄—CH₃ or —C₆H₄—CH₂CH(CH₃).

“Substituted alkyl”, “substituted cycloalkyl”, “substituted alkenyl”, “substituted alkynyl”, substituted alkylaryl”, “substituted arylalkyl”, “substituted heterocycle”, “substituted aryl”, “substituted monosaccharide” and the like mean an alkyl, alkenyl, alkynyl, alkylaryl, arylalkyl heterocycle, aryl, monosaccharide or other group or moiety as defined or disclosed herein that has a substituent(s) that replaces a hydrogen atom(s) or a substituent(s) that interrupts a carbon atom chain. Alkenyl and alkynyl groups that comprise a substituent(s) are optionally substituted at a carbon that is one or more methylene moiety removed from the double bond.

“Optionally substituted alkyl”, “optionally substituted alkenyl”, “optionally substituted alkynyl”, “optionally substituted alkylaryl”, “optionally substituted arylalkyl”, “optionally substituted heterocycle”, “optionally substituted aryl”, “optionally substituted heteroaryl”, “optionally substituted alkylheteroaryl”, “optionally substituted heteroarylalkyl”, “optionally substituted monosaccharide” and the like mean an alkyl, alkenyl, alkynyl, alkylaryl, arylalkyl heterocycle, aryl, heteroaryl, alkylheteroaryl, heteroarylalkyl, monosaccharide or other group or moiety as defined or disclosed herein that has a substituent(s) that optionally replaces a hydrogen atom(s) or a substituent(s) that interrupts a carbon atom chain. Such substituents are as described above. For a phenyl moiety, the arrangement of any two substituents present on the aromatic ring can be ortho (o), meta (m), or para (p).

For any group or moiety described by a given range of carbon atoms, the designated range means that any individual number of carbon atoms is described. Thus, reference to, e.g., “C₁-C₄ optionally substituted alkyl”, “C₂₋₆ alkenyl optionally substituted alkenyl”, “C₃-C₈ optionally substituted heterocycle” specifically means that a 1, 2, 3 or 4 carbon optionally substituted alkyl moiety as defined herein is present, or a 2, 3, 4, 5 or 6 carbon alkenyl, or a 3, 4, 5, 6, 7 or 8 carbon moiety comprising a heterocycle or optionally substituted alkenyl moiety as defined herein is present. All such designations are expressly intended to disclose all of the individual carbon atom groups and thus “C1-C4 optionally substituted alkyl” includes, e.g., 3 carbon alkyl, 4 carbon substituted alkyl and 4 carbon alkyl, including all positional isomers and the like are disclosed and can be expressly referred to or named.

The organic moieties and substitutions described here, and for other any other moieties described herein, usually will exclude unstable moieties except where such unstable moieties are transient species that one can use to make a compound with sufficient chemical stability for the one or more of the uses described herein.

The term “phosphorous containing moiety” means a moiety that contains a phosphorous atom that is covalently bonded to a molecule or entity, such as a polypeptide or a biopolymer material, through a carbon atom or a heteroatom, such as O, N or S, of the molecule or entity. Typically a phosphorous containing moiety will have a P═O bond and includes, by way of example and not limitation, moieties such as —P(O)(O)—OR^{PR}, —P(O)(R)(OR^{PR}), —P(O)(OR^{PR})(OR^{PR}), wherein R^{PR} are independently selected —H or an organic moiety containing 1-50 carbon atoms, 1-20 carbon atoms or 1-8 carbon atoms and 0 to 10 independently selected heteroatoms (e.g., O, S, N, P, Si), typically 0-2. In some embodiments the phosphorous containing moiety is derived form an organophosphate compound, or an organophosphoryl compound having the structure 1b or 2 by combining with a heteroatom from a molecule or entity. In some embodiments the phosphorous containing moiety is covalently bonded through a nitrogen or oxygen of a polypeptide to provide a biomarker.

“Heterocycle” or “heterocyclic” as used here is a cycloalkyl or aromatic ring system wherein one or more, typically 1, 2 or 3, but not all of the carbon atoms comprising the ring system are replaced by a heteroatom which is an atom other than carbon, including, N, O, S, Se, B, Si, P, typically N, O or S wherein two or more heteroatoms may be adjacent to each other or separated by one or more carbon atoms, typically 1-17 carbon atoms, 1-7 atoms or 1-3 atoms.

The term C-linked heterocycle means a heterocycle that is bonded to a molecule through a carbon atom and include moieties such as —(CH₂)ₙ-heterocycle where n is 1, 2 or 3 or —C<heterocycle where C< represents a carbon atom in a heterocycle ring. Moieties that are N-linked heterocycles mean a heterocycle that is bonded a heterocycle ring nitrogen described as —N<heterocycle where N< represents a nitrogen.

“Heteroaryl” as used here means an aryl ring system wherein one or more, typically 1, 2 or 3, but not all of the carbon atoms comprising the aryl ring system are replaced by a heteroatom which is an atom other than carbon, including, N, O, S, Se, B, Si, P, typically, usually oxygen (—O—), nitrogen (—NX—) or sulfur (—S—) where X is —H, a protecting group or C₁₋₆ optionally substituted alkyl, wherein the heteroatom participates in the conjugated system either through pi-bonding with an adjacent atom in the ring system or through a lone pair of electrons on the heteroatom and may be optionally substituted on one or more carbons or heteroatoms, or a combination of both, comprising the heterocycle in a manner which retains the cyclically conjugated system. Examples are as described for heterocycle.

Heterocycles and heteroaryls, includes by way of example and not limitation, heterocycles and heteroaryls described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 1960, 82:5545-5473 particularly 5566-5573). Examples of heteroaryls include by way of example and not limitation pyridyl, thiazolyl, pyrimidinyl, furanyl, thienyl, pyrrolyl, pyrazolyl, purinyl, imidazolyl, benzofuranyl, indolyl, isoindoyl, quinolinyl, isoquinolinyl, benzimidazolyl, pyridazinyl, pyrazinyl, benzothiopyran, benzotriazine, isoxazolyl, pyrazolopyrimidinyl, quinoxalinyl, thiadiazolyl, triazolyl and the like. Examples of heterocycles that are not heteroaryls include by way of example and not limitation tetrahydrothiophenyl, tetrahydrofuranyl, indolenyl, piperidinyl, pyrrolidinyl, 2-pyrrolidonyl, tetrahydroquinolinyl, tetrahydroisoquinolinyl, decahydroquinolinyl, octahydroisoquinolinyl, 2H-pyrrolyl, 3H-indolyl, 4H-quinolizinyl, imidazolidinyl, imidazolinyl, pyrazolidinyl, piperazinyl, quinuclidinyl, morpholinyl, oxazolidinyl and the like.

“Alkylheteroaryl” as used here means a moiety where an alkyl group is bonded to a heteroaryl group, i.e., -alkyl-heteroaryl, where alkyl and heteroaryl groups are as described above.

“Heteroarylalkyl” as used here means a moiety where an heteroaryl group is bonded to an alkyl group, i.e., -heteroaryl-alkyl, where heteroaryl and alkyl groups are as described above.

“Alcohol” as used herein means an alcohol that comprises a C₁₋₁₂ alkyl moiety substituted at a hydrogen atom with one hydroxyl group. Alcohols include methanol, ethanol, n-propanol, i-propanol, n-butanol, i-butanol, s-butanol and t-butanol. The carbon atoms in alcohols can be straight, branched or cyclic. Alcohol includes any subset of the foregoing, e.g., C₁₋₄ alcohol (or C2-4 alcohol), meaning an alcohol having 1, 2, 3 or 4 carbon atoms or C₂₋₈ alcohol or (C2-8 alcohol), meaning an alcohol having 2, 3, 4, 5, 6, 7 or 8 carbon atoms.

“Halogen” or “halo” as used here means fluorine, chlorine, bromine or iodine.

“Protecting group” as used here means a moiety that prevents or reduces the ability of the atom or functional group to which it is linked from participating in unwanted reactions. For example, for —OR^{PR}, R^{PR} may be hydrogen or a protecting group for the oxygen atom found in a hydroxyl, while for —C(O)—OR^{PR}, R^{PR} may be hydrogen or a carboxylic acid protecting group; for —SR^{PR}, R^{PR} may be hydrogen or a protecting group for sulfur in thiols and for —NHR^{PR} or —N(R^{PR})₂—, R^{PR} may be hydrogen or a nitrogen atom protecting group for primary or secondary amines. Hydroxyl, amine, ketones and other reactive groups may require protection against reactions taking place elsewhere in the molecule. The protecting groups for oxygen, sulfur or nitrogen atoms are usually used to prevent unwanted reactions with electrophilic compounds, such as acylating agents. Typical protecting groups for atoms or functional groups are given in Greene (1999), “Protective groups in organic synthesis, 3^{rd} ed.” Wiley Interscience.

“Ester” as used here means a moiety that contains a —C(O)—O— structure wherein the carbon atom of the structure is not directly connected to another heteroatom and is directly connected to —H or another carbon atom. Typically, esters as used here comprise an organic moiety containing 1-50 carbon atoms, 1-20 carbon atoms or 1-8 carbon atoms and 0 to 10 independently selected heteroatoms (e.g., O, S, N, P, Si), typically 0-2 where the organic moiety is bonded through the —C(O)—O— structure and include ester moieties such as organic moiety-C(O)—O— and organic moiety-O—C(O)—. The organic moiety usually comprises one or more of any of the organic groups described herein, e.g., C₁₋₂₀ alkyl moieties, C₂₋₂₀ alkenyl moieties, C₂₋₂₀ alkynyl moieties, aryl moieties, C₂₋₉ heterocycles or substituted derivatives of any of these, e.g., comprising 1, 2, 3, 4 or more substituents, where each substituent is independently chosen. Exemplary substitutions for hydrogen or carbon atoms in these organic groups are as described above for substituted alkyl and other substituted moieties and are independently chosen. The substitutions listed above are typically substituents that one can use to replace one or more carbon atoms, e.g., —O— or —C(O)—, or one or more hydrogen atom, e.g., halogen, —NH₂ or —OH. Exemplary esters include by way of example and not limitation, one or more independently selected acetate, propionate, isopropionate, isobutyrate, butyrate, valerate, isovalerate, caproate, isocaproate, hexanoate, heptanoate, octanoate, phenylacetate or benzoate esters. Ester also includes ester moieties such as polypeptide-O—C(O)—, polymer-O—C(O)—, —O—C(O)-polypeptide or —O—C(O)-polymer.

“Thioester” as used here means a moiety that contains a —C(O)—S— structure. Typically, thioesters comprise an organic moiety containing 1-50 carbon atoms, 1-20 carbon atoms or 1-8 carbon atoms and 0 to about 10 independently selected heteroatoms (e.g., O, S, N, P, Si), typically 0-2, where the organic moiety is bonded through the —C(O)—S— structure and include thioester moieties such as organic moiety-C(O)—S— and —C(O)—S-organic moiety where the organic moiety is as described herein for ester, optionally substituted alkyl or alkyl group. Thioester also includes thioester moieties such as polypeptide-C(O)—S—, polymer-C(O)—S—, —C(O)—S-polypeptide or —C(O)—S-polypeptide.

“Thionoester” as used here means a moiety that contains a —C(S)—O— structure. Typically, thionoesters comprise an organic moiety containing about 1-50 carbon atoms (e.g., about 1-20 carbon atoms) and 0 to about 10 independently selected heteroatoms (e.g., O, S, N, P, Si) where the organic moiety is bonded through the —C(O)—S— structure and include thionoester moieties such as organic moiety-C(S)—O—, organic moiety-O—C(S)—, where the organic moiety is as described herein for esters, alkyl groups and optionally substituted alkyl groups. Thionoester also includes thionoester moieties such as —C(S)—O-polypeptide, polypeptide-C(S)—O—, polymer-C(S)—O— or —C(S)—O-polymer.

“Acetal”, “thioacetal”, “ketal”, “thioketal” and the like as used here is a moiety having a carbon to which is bonded two of the same or different heteroatoms wherein the heteroatoms are independently selected S and O. For acetal the carbon has two bonded oxygen atoms, a hydrogen atom and an organic moiety. For ketal, the carbon has two bonded oxygen atoms and two independently selected organic moieties where the organic moiety is as described herein for ester, alkyl or optionally substituted alkyl group. For thioacetals and thioketals one or both of the oxygen atoms in acetal or ketal, respectively, is replaced by sulfur. The oxygen or sulfur atoms in ketals and thioketals are sometimes linked by an optionally substituted alkyl moiety. Typically, the alkyl moiety is an optionally substituted C₁₋₈ alkyl or branched alkyl structure such as —C(CH₃)₂—, —CH(CH₃)—, —CH₂—, —CH₂—CH₂—, —C[(C2-C4 alkyl)₂]_{1, 2, 3}- or —[CH(C2-C4 alkyl)]_{1, 2, 3}-. Some of these moieties can serve as protecting groups for an aldehyde or ketone, e.g., acetals for aldehydes and ketals for ketones and contain —O—CH₂—CH₂—CH₂—O— or —O—CH₂—CH₂—O— moieties that form a spiro ring with the carbonyl carbon, and can be removed by chemical synthesis methods or by metabolism in cells or biological fluids.

“Phosphoester” or “phosphate ester” as used here means a moiety that contains a —O—P(OR^{PR})(O)—O—, —O—P(O)(OR^{PR})—OR^{PR}, or —O—P(O)(OR^{PR})—O— structure or a salt thereof, where R^{PR} independently are —H, a protecting group or an organic moiety where the organic moiety is as described herein for ester, alkyl or optionally substituted alkyl group. Typically, phosphoesters comprise a hydrogen atom, a protecting group or an organic moiety containing 1-50 carbon atoms, 1-20 carbon atoms or 1-8 carbon atoms and 0 to about 10 independently selected heteroatoms (e.g., O, S, N, P, Si), typically 0-2, bonded through the —O—P(O)(O)—O— structure, e.g., organic moiety-O—P(O)(OH)—O— where the organic moiety is as described for ester, optionally substituted alkyl or alkyl group. Exemplary phosphoesters include —O—P(O)(OH)—O—CH₃, —O—P(O)(OCH₃)—O—CH₃, —O—P(O)(OH)—O—CH₂—CH₃, —O—P(O)(OC₂H₅)—O—CH₂—CH₃, —O—P(O)(OH)—O—CH₂—CH₂—CH₃, —O—P(O)(OH)—O—CH(CH₃)—CH₃, —O—P(O)(OH)—O—CH₂—CH₂—CH₂—CH₃, —O—P(O)(O(CH₃)₃)—O—C(CH₃)₃, —O—P(O)(OH)—O—C(CH₃)₃, —O—P(O)(O-optionally substituted alkyl)-OR^{PR} and —O—P(O)(O-optionally substituted alkyl)-O-optionally substituted alkyl, where optionally substituted alkyl moieties are independently chosen. Phosphoesters also include phosphoester moieties such as a polypeptide-O—P(OR^{PR})(O)—O, polypeptide-O—P(O)(OR^{PR})—OR^{PR}, polymer-O—P(OR^{PR})(O)—O— or polymer-O—P(O)(OR^{PR})—OR^{PR}.

“Phosphonate”, “phosphonate ester” or the like as used here means a moiety that contain a —O—P(O)(OR^{PR})— or a —O—P(O)(O-optionally substituted alkyl)- structure or a salt thereof having a carbon atom directly attached to the phosphorous atom of the structure, wherein R^{PR} independently are —H, a protecting group or an organic moiety as described for esters, optionally substituted alkyl or alkyl group. Typically, phosphonates or phosphonate esters comprise a hydrogen atom, a protecting group or an organic moiety containing 1-50 carbon atoms, 1-20 carbon atoms or 1-8 carbon atoms and 0 to 10 independently selected heteroatoms (e.g., O, S, N, P, Si), typically 0-2, bonded through —P(O)(O)—, e.g., organic moiety-P(O)(OH)—O—, —P(O)(OR^{PR})—O-organic moiety or —O—P(O)(OR^{PR})—C₁₋₈ optionally substituted alkyl where the organic moiety and optionally substituted alkyl is as described for esters, optionally substituted alkyl or alkyl group. Exemplary phosphonate esters include —O—P(O)(OH)—CH₃, —O—P(O)(OCH₃)—CH₃, —O—P(O)(OH)—CH₂—CH₃, —O—P(O)(OC₂H₅)—CH₂—CH₃, —O—P(O)(OH)—CH₂—CH₂—CH₃, —O—P(O)(OH)—CH(CH₃)—CH₃, —O—P(O)(OH)—CH₂—CH₂—CH₂—CH₃, —O—P(O)(O(CH₃)₃)—C(CH₃)₃, —O—P(O)(OH)—C(CH₃)₃, —O—P(O)(OR^{PR})-optionally substituted heteroaryl, —O—P(O)(O-optionally substituted alkyl)-optionally substituted alkyl, —P(O)(OH)—OCH₃, —P(O)(OCH₃)—OCH₃, —P(O)(OH)—OCH₂—CH₃, —P(O)(OC₂H₅)—OCH₂—CH₃, —P(O)(OR^{PR})—O—C₁₋₈ optionally substituted alkyl, —O—P(O)(OR^{PR})-optionally substituted aryl, —P(O)(OR^{PR})—O-optionally substituted aryl, —O—P(O)(OR^{PR})—C₆H₅—P(O)(OR^{PR})—O—C₆H₅, —O—P(O)(OC₂H₅)—C₁₋₈ optionally substituted alkyl, —P(O)(O—C₁₋₈ optionally substituted alkyl)-O—C₁₋₈ optionally substituted alkyl, where optionally substituted alkyl moieties are independently chosen. Phosphonate also includes phosphonate moieties such as polypeptide-O—P(O)(OR^{PR})—, polypeptide-O—P(O)(O-optionally substituted alkyl)-, polymer-O—P(O)(OR^{PR})—, polymer-O—P(O)(O-optionally substituted alkyl)-, —O—P(O)(OR^{PR})-polypeptide, —O—P(O)(O-optionally substituted alkyl)-polypeptide, —O—P(O)(OR^{PR})-polymer or —O—P(O)(O-optionally substituted alkyl)-polymer.

“Phosphothioester” or “thiophosphate” as used here means a moiety that contains a —O—P(SR^{PR})(O)—O—, —O—P(O)(OR^{PR})—S—, —O—P(O)(SR^{PR})—O—, —O—P(O)(SR^{PR})—O-optionally substituted alkyl structure or a salt thereof where R^{PR} is —H, a protecting group or an organic moiety as described for esters, optionally substituted alkyl or alky group. Typically, phosphothioesters as used here comprise a hydrogen atom, a protecting group or an organic moiety containing 1-50 carbon atoms, 1-20 carbon atoms or 1-8 carbon atoms and 0 to 10 independently selected heteroatoms (e.g., O, S, N, P, Si), typically 0-2, bonded through —O—P(O)S— or —O—P(SH)—O—, e.g., organic moiety-O—P(O)(SH)—O— or —O—P(O)(OR^{PR})—S-organic moiety where the organic moiety is described herein for ester, alkyl or optionally substituted alkyl group. Exemplary phosphothioesters are as described for phosphoesters, except that sulfur replaces the appropriate oxygen atom. Phosphothioester also include phosphothioester moieties such as polypeptide-O—P(O)(SH)—O—, —O—P(O)(OR^{PR})—S-polypeptide, polymer-O—P(O)(SH)—O—, —O—P(O)(OR^{PR})—S-polymer.

“Phosphoramidate”, “phosphoramidate ester” or the like as used here means a moiety that contains a —O—P(O)(N(R^{PR})₂)—O—, —O—P(O)(OR^{PR})N(R^{PR})—, —O—P(O)(N(optionally substituted alkyl)₂)—O—, —O—P(O)(O-optionally substituted alkyl)N(optionally substituted alkyl)-, or a salt thereof, with optionally substituted alkyl groups independently selected, where R^{PR} is —H, a protecting group, an organic moiety as described for esters, alkyl groups or optionally substituted alkyl groups. Phosphoamidates or phosphoamidate esters as used herein may comprise a hydrogen atom, a protecting group or an organic moiety containing 1-50 carbon atoms, 1-20 carbon atoms or 1-8 carbon atoms and 0 to 10 independently selected heteroatoms (e.g., O, S, N, P, Si), typically 0-2, bonded through a suitable structure such as —O—P(O)(N(R^{PR})₂)—O— or —O—P(O)(OR^{PR})N— and include phosphoramidates or phosphoramidate moieties such as organic moiety-O—P(O)(N(R^{PR})₂)—O—, —O—P(O)(OR^{PR})N(R^{PR})-organic moiety, where R^{PR} and organic moiety are independently selected and where the organic moiety is as described herein for ester, alkyl or optionally substituted alkyl group. Phosphoamidates also include phosphoamidate moieties such as polypeptide-O—P(O)(N(R^{PR})₂)—O—, —O—P(O)(OR^{PR})N(R^{PR})-polypeptide, polymer-O—P(O)(N(R^{PR})₂)—O— or —O—P(O)(OR^{PR})N(R^{PR})-polymer.

“Thiophosphonate”, “thiophosphonate ester” and the like as used here means a moiety that contains a —O—P(S)(OR^{PR}) structure where R^{PR} is —H, a protecting group or an organic moiety as described for esters, alkyl groups or optionally substituted alkyl groups. Typically, thiophosphonate esters as used here comprise a protecting group or an organic moiety containing 1-50 carbon atoms, 1 to 20 carbon atoms or 1-8 carbon atoms and 0 to about 10 independently selected heteroatoms (e.g., O, S, N, P, Si), typically 0-2, bonded through a suitable structure suitable structure such as —O—P(S)(OR^{PR})— and include organic moiety-P(S)(OR^{PR})—O— or —P(S)(OR^{PR})(O)-organic moiety where R^{PR} is as previously described and the organic moiety is as described herein for ester, alkyl or optionally substituted alkyl group. Exemplary thiophosphothioesters are as described for phosphonates except that sulfur replaces the appropriate oxygen atom. Thiophosphonates also include moieties such as polypeptide-P(S)(OR^{PR})—O—, —P(S)(OR^{PR})—O-polypeptide, polymer-P(S)(OR^{PR})—O— or —P(S)(OR^{PR})—O-polymer.

“Sulfate ester” as used here means a moiety that contains a —O—S(O)(O)—O— structure. Typically, sulfate esters as used here comprise a hydrogen atom, a protecting group or an organic moiety containing 1-50 carbon atoms, 1 to 20 carbon atoms or 1 to 8 carbon atoms and 0 to 10 independently selected heteroatoms (e.g., O, S, N, P, Si), typically 0-2, bonded through —O—S(O)(O)—O—, e.g., organic moiety-O—S(O)(O)—O— where the organic moiety is as described herein for ester, alkyl or optionally substituted alkyl group. Sulfate esters include —O—S(O)(O)—O-optionally substituted alkyl, —O—S(O)(O)—O—CH₃, —O—S(O)(O)—O-optionally substituted aryl, —O—S(O)(O)—O-optionally substituted heteroaryl, —O—S(O)(O)—O—C₆H₅ and the like. Sulfate ester also includes sulfate ester moieties such as polypeptide-O—S(O)(O)—O— or polymer-O—S(O)(O)—O—.

“Sulfamate ester”, “sulfamate derivative”, “sulfamate” and the like as used here means a moiety that contains a —O—S(O)(O)—NH—, —O—S(O)(O)—NH₂, —O—S(O)(O)—NH-optionally substituted alkyl or —O—S(O)(O)—N-(optionally substituted alkyl)₂ structure, where each optionally substituted alkyl moiety is independently selected. Typically, sulfamate derivatives as used here comprise an organic moiety containing 1-50 carbon atoms, 1-20 atoms or 1-8 carbon atoms and 0 to 10 independently selected heteroatoms (e.g., O, S, N, P, Si), typically 0-2, bonded through —O—S(O)(O)—N— and include moieties such as organic moiety-O—S(O)(O)—NH—, —O—S(O)(O)—NH-organic moiety, —O—S(O)(O)—NH—C₁₋₈ alkyl, —O—S(O)(O)—N(C₁₋₈ alkyl)₂, —O—S(O)(O)—NHR^{PR}, —NH—S(O)(O)—OH or —O—S(O)(O)—NH₂, where alkyl groups are independently chosen and the organic moiety is as described herein for ester, alkyl or optionally substituted alkyl moiety. Sulfamate also includes sulfamate moieties such as polypeptide-O—S(O)(O)—NH—, —O—S(O)(O)—NH-polypeptide, polymer-O—S(O)(O)—NH— or —O—S(O)(O)—NH-polymer.

“Sulfamide” and the like as used here means a moiety that contains a —NH—S(O)(O)—NH— or —NH—S(O)(O)—NH₂ structure. Typically, sulfamide moieties comprise an organic moiety containing 1-50 carbon atoms, 1-20 carbon atoms or 1-8 carbon atoms and 0 to 10 independently selected heteroatoms (e.g., O, S, N, P, Si), typically 0-2, bonded through —NH—S(O)(O)—NH—, e.g., —NH—S(O)(O)—NH-organic moiety, —NH—S(O)(O)—NH₂, —NH—S(O)(O)—NHR^{PR} or —NH—S(O)(O)—N(R^{PR})₂, where R^{PR} independently or together are a protecting group such as C₁₋₈ optionally substituted alkyl and the organic moiety is as described herein for ester, alkyl or optionally substituted alkyl group.

“Sulfinamide” and the like as used here means a moiety that comprises a —C—S(O)—NH— structure. Typically, sulfinamide moieties comprise an organic moiety containing 1-50 carbon atoms, 1 to 20 carbon atoms or 1-8 carbon atoms and 0 to 10 independently selected heteroatoms (e.g., O, S, N, P, Si), typically 0-2, bonded through a suitable structure such as —S(O)—NH-organic moiety, —NH—S(O)-organic moiety, organic moiety-S(O)—NH₂, organic moiety-S(O)—NHR^{PR} or organic moiety-S(O)—N(R^{PR})₂, where R^{PR} independently or together are a protecting group such as C₁₋₈ optionally substituted alkyl and the organic moiety is as described herein for ester, alkyl or optionally substituted alkyl group.

“Sulfurous diamide” and the like as used here means a moiety that comprises a —NH—S(O)—NH— or —NH—S(O)—NH₂ structure. Typically, sulfurous diamide moieties comprise an organic moiety containing 1-50 carbon atoms, 1 to 20 carbon atoms or 1-8 carbon atoms and 0 to 10 independently selected heteroatoms (e.g., O, S, N, P, Si), typically 0-2, bonded through —NH—S(O)—NH— e.g., —NH—S(O)—NH-organic moiety, —NH—S(O)—NH₂, —NH—S(O)—NHR^{PR} or —NH—S(O)—N(R^{PR})₂, where R^{PR} independently or together are a protecting group such as C₁₋₈ optionally substituted alkyl and the organic moiety is as described herein for ester, alkyl or optionally substituted alkyl group. Sulfurous diamide includes sulfurous diamide moieties such as polypeptide-NH—S(O)—NH— or polymer-NH—S(O)—NH—.

“Sulfonate ester”, “sulfonate derivative”, “sulfonate” and the like as used here means a moiety that comprises a —O—S(O)(O)— or —S(O)(O)—OR^{PR} structure and a carbon atom directly attached to the sulfur atom of the structure where R^{PR} is —H or a protecting group. Typically, sulfonate derivatives comprise an organic moiety containing 1-50 carbon atoms, 1-20 carbon atoms or 1-8 carbon atoms and 0 to 10 independently selected heteroatoms (e.g., O, S, N, P, Si), typically 0-2, bonded through —S(O)(O)—O—, e.g., —S(O)(O)—O-organic moiety, where the organic moiety is as described herein for ester, alkyl or optionally substituted alkyl group, —S(O)(O)—O—C₁₋₈ optionally substituted alkyl, —O—S(O)(O)—C₁₋₈ optionally substituted alkyl, —O—S(O)(O)-heteroaryl, —S(O)(O)—O-aryl or —S(O)(O)—O-heteroaryl where the aryl or heteroaryl moiety is optionally substituted with 1, 2, 3, 4 or 5 independently selected substitutions, —O—S(O)(O)-organic moiety, where the organic moiety is as described herein for ester, alkyl or optionally substituted alkyl group, —O—S(O)(O)-heteroaryl or —O—S(O)(O)-aryl, where the aryl or heteroaryl moiety is optionally substituted with 1, 2, 3, 4 or 5 independently selected substitutions, —O—S(O)(O)—CH₃, —O—S(O)(O)—C₆H₅ and the like. Sulfate ester also includes sulfate ester moieties such as polypeptide-O—S(O)(O)—, —O—S(O)(O)—, or polymer-O—S(O)(O)—.

“Sulfonamide” as used here means a moiety that contain a —S(O)N(R^{PR})₂, —S(O)N(optionally substituted alkyl)-, or a —S(O)N(optionally substituted alkyl)₂ structure and a carbon atom directly attached to the sulfur atom of the structure, where R^{PR} and optionally substituted alkyl are independently selected and R^{PR} are —H, a protecting group or an organic moiety as described herein for esters, optionally substituted alkyl or alkyl group. Typically, sulfonamides comprise a protecting group or an organic moiety containing 1-50 carbon atoms, 1-20 carbon atoms or 1-8 carbon atoms and 0 to about 10 independently selected heteroatoms (e.g., O, S, N, P, Si), typically 0-2, bonded through a suitable structure such as —S(O)N(R^{PR})— e.g., organic moiety-S(O)N(R^{PR})— or —S(O)N(R^{PR})-organic moiety, where the organic moiety and is as described herein for esters, optionally substituted alkyl or alkyl group and R^{PR} is previously described. Exemplary sulfonamides include C₁₋₈ optionally substituted alkyl S(O)N(R^{PR})—, aryl-S(O)N(R^{PR})—, heteroaryl-S(O)N(R^{PR})—, where the aryl or heteroaryl moiety is optionally substituted with 1, 2, 3, 4 or 5 independently selected substitutions and R^{PR} is previously described or C₆H₅—S(O)NH—. Sulfonamides also include sulfonamide moieties such as polypeptide-NH—S(O)—, polymer-NH—S(O)— or polymer-S(O)NH—. Sulfonamides are typically prepared by condensing a sulfonyl chloride with a molecule having a primary or secondary amine group.

“Amide”, “amide derivative” and the like as used here means an moiety that contains a —C(O)—NR^{PR}— or —C(O)—NH— structure with no other heteroatom directly attached to the carbon of the structure and where R^{PR} is —H, a protecting group or an organic moiety where the organic moiety is as described herein for ester, alkyl or optionally substituted alkyl group. Typically, amide derivatives comprise an organic moiety containing 1-50 carbon atoms, 1-20 carbon atoms or 1-8 carbon atoms and 0 to 10 independently selected heteroatoms (e.g., O, S, N, P, Si), typically 0-2, bonded through a suitable structure such as —C(O)NR^{PR}—. In some embodiments, the —C(O)NR^{PR}— group is organic moiety-C(O)NR organic moiety-C(O)—NH— or —C(O)NR^{PR}-organic moiety where R^{PR} and organic moiety are independently selected and the organic moiety is as described herein for ester, alkyl or optionally substituted alkyl group and R^{PR} is previously described. Amide also includes amide moieties such as —C(O)^{NR}-polypeptide, —C(O)NH-polypeptide, polymer-C(O)NR^{PR}—, polymer-C(O)—NH— or —C(O)NR^{PR}-polymer. Amides are prepared by condensing an acid halide, such an acid chloride with a molecule containing a primary or secondary amine. Alternatively amide coupling reactions well known in the art of peptide synthesis, which oftentimes proceed through an activated ester of a carboxylic acid-containing molecule, are used. Examples for preparing amide bonds is provided in Benoiton (2006) Chemistry of peptide synthesis CRC Press, Bodansky (1988) Peptide synthesis: A practical textbook, Springer-Verlag, Frinkin, M. et al. (1974) Peptide synthesis, Ann. Rev. Biochem. 43:419-443. Reagents used in the preparation of activated carboxylic acids is provided in Han, et al. (2004) Recent development of peptide coupling agents in organic synthesis, Tet. 60:2447-2476.

“Ether” as used here means an organic moiety that comprises 1, 2, 3, 4 or more —O— moieties, usually 1 or 2, wherein no two —O— moieties are immediately adjacent (i.e., directly attached) to each other. Typically, ether derivatives comprise an organic moiety containing 1-50 carbon atoms, 1-20 carbon atoms or 1-8 carbon atoms and 0 to 10 independently selected heteroatoms (e.g., O, S, N, P, Si), typically 0-2. Ether moiety includes organic moiety-O— where the organic moiety is as described herein for ester, alkyl or optionally substituted alkyl group. Ether also includes ether moieties such as polypeptide-O— or polymer-O—.

“Thioether” as used here means an organic moiety as described for ester, optionally substituted alkyl or alkyl group that comprises 1, 2, 3, 4 or more —S— moieties, usually 1 or 2, wherein no two —S— moieties are immediately adjacent to one another. Thioether moieties include organic moiety-S—, organic moiety-S—CH₂—S— where the organic moiety is as described herein for ester, alkyl or optionally substituted alkyl group. Thioether includes thioether moieties such as polypeptide-S— or polymer-S—.

“Disulfide” as used herein means an organic moiety that comprises a —S—S— or —S—S—R^{PR} structure where R^{PR} is —H, a protecting group or an organic moiety where the organic moiety is as described herein for ester, alkyl or optionally substituted alkyl group. Typically, disulfide derivatives comprise an organic moiety containing about 1-50 carbon atoms, 1-20 carbon atoms or 1-8 carbon atoms and 0 to 10 independently selected heteroatoms (e.g., O, S, N, P, Si), typically 0-2, linked through a suitable structure such as —S—S—, e.g., organic moiety-S—S—, where the organic moiety is as described herein for ester, alkyl or optionally substituted alkyl group, —S—S—C₁₋₈ optionally substituted alkyl, —S—S-aryl or —S—S-heteroaryl, where the aryl or heteroaryl moiety is optionally substituted with 1, 2, 3, 4 or 5 independently selected substitutions. Disulfide also includes disulfide moieties such as polypeptide-S—S— or polymer-S—S—. Sometimes a disulfide moiety such as polypeptide-S—S— is prepared using a sulfhydryl group of a cysteine amino acid residue that comprises a polypeptide and another sulfhydryl containing molecule or is prepared by exchange of a sulfhydryl moiety in a disulfide containing compound with another sulfhydryl moiety.

“Hydrazide” as used herein means an organic moiety that contains a —C(O)N(R^{PR})—N(R^{PR})—, —C(O)N(R^{PR})—NH—, —C(O)NH—N(R^{PR})₂— or —C(O)NH(R^{PR})NH₂, where R^{RP} are independently —H, a protecting group or an organic moiety where the organic moiety is described herein for ester, alkyl or optionally substituted alkyl group. Typically, hydrazone derivatives comprise an organic moiety containing 1-50 carbon atoms, 1-20 carbon atoms or 1-8 carbon atoms and 0 to 10 independently selected heteroatoms (e.g., O, S, N, P, Si), typically 0-2, bonded through a suitable structure such as —C(O)NH—NH— or —C(O)N(R^{PR})NH, e.g., organic moiety-C(O)NH—NH—, —C(O)NH—NH(organic moiety), —C(O)NH—NH(organic moiety)₂, where organic moiety is independently selected and is as described herein for ester, alkyl or optionally substituted alkyl group and where R^{PR} is previously described. Hydrazide also includes hydrazide moieties such as polymer-C(O)NH—NH—, —C(O)NH—NH-polymer or polypeptide-C(O)NH—NH—. Typically, hydrazones are formed by condensing a hydrazine with an entity or a molecule containing a carboxylic acid derivative such as an acid chloride or an activated carboxylic acid ester.

“Hydrazone” as used herein means an organic moiety that contains a >C═N—N(R^{PR})₂, >C═N—N(R^{PR})(optionally substituted alkyl), >C═N—N(optionally substituted alkyl)₂ or >C═N—N— structure where >C represents a carbon atom and —H substituents or two other carbon atom substituents attached and where R^{RP} and optionally substituted alkyl are independently selected and R^{PR} independently are —H, a protecting group or an organic moiety where the organic moiety is as described herein for ester, alkyl or optionally substituted alkyl group. Typically, hydrazone derivatives comprise an organic moiety containing about 1-50 carbon atoms, 1-20 carbon atoms or 1-8 carbon atoms and 0 to 10 independently selected heteroatoms (e.g., O, S, N, P, Si), typically 0-2, linked through a suitable structure such as —C(═N—N(R^{PR}))— or >C═N—N—, e.g., organic moiety-C(═N—NH₂)— or >C═N—NH-organic moiety, where the organic moiety is described herein for ester, alkyl or optionally substituted alkyl group. Hydrazone also includes hydrazone moieties such as >C(O)NH—NH-polypeptide or >C(O)NH—NH-polymer. Hydrazones are sometimes prepared by condensing an aldehyde or a ketone with a molecule containing a hydrazine or hydrazide having the structure >C—NHNH₂ or >C(O)NH—NH₂, where >C oftentimes represents a carbon atom having two carbon atoms attached, or through exchange of carbonyl moieties between two different hydrazone containing molecules. Sometimes an aldehyde is introduced into a polypeptide and is condensed with a hydrazine or hydrazide containing molecule to form a hydrazone.

“Acyl group” or “acyl” as used here means an moiety that contain a —C(O)— group when the moiety is attached to a heteroatoms such as S or O. In some embodiments, the acyl moiety is organic moiety-C(O)—.

“Thioacyl” as used here means an organic moiety as described for ester that comprises a —C(S)— groups when attached to a heteroatoms such as S or O. In some embodiments, the —C(S)— group is organic moiety-C(S)— where the organic moiety is as described for ester, optionally substituted alkyl or alkyl group.

“Carbonate” as used here means a moiety that contains a —O—C(O)—O— structure. Typically, carbonate groups as used here comprise an organic moiety containing 1-50 carbon atoms, 1-20 carbon atoms or 1-8 carbon atoms and 0 to 10 independently selected heteroatoms (e.g., O, S, N, P, Si), typically 0-2, bonded through the —O—C(O)—O— structure, e.g., organic moiety-O—C(O)—O. Carbonate also includes carbonate moieties such as polypeptide-O—C(O)—O— or polymer-O—C(O)—O—.

“Carbamate” or “urethane” as used here means an organic moiety that contains a —O—C(O)N(R^{PR})—, —O—C(O)N(R^{PR})₂, —O—C(O)NH(optionally substituted alkyl) or C(O)N(optionally substituted alkyl)-2-structure where R^{PR} and optionally substituted alkyl are independently selected and R^{PR} are independently —H, a protecting group or an organic moiety as described for ester, alkyl or optionally substituted alkyl. Typically, carbamate groups as used here comprise an organic moiety containing about 1-50 carbon atoms, 1-20 carbon atoms or 1-8 carbon atoms and 0 to 10 independently selected heteroatoms (e.g., O, S, N, P, Si), typically 0-2, bonded through the —O—C(O)—NR^{PR}— structure, e.g., organic moiety-O—C(O)—NR^{PR}— or —O—C(O)—NR^{PR}-organic moiety. Carbamate also include carbamate moieties such as polypeptide-O—C(O)—NR^{PR}—, —O—C(O)—NR^{PR}-polypeptide, polymer-O—C(O)—NR^{PR}— or —O—C(O)—NR^{PR}-polymer.

“Urea” as used here means an organic moiety that contains a —N(R^{PR})—C(O)—N(R^{PR})—, —N(optionally substituted alkyl)-C(O)—N(optionally substituted alkyl)-, —NH—C(O)N(optionally substituted alkyl)₂-, —N (optionally substituted alkyl)-C(O)N(optionally substituted alkyl)₂- structure where R^{PR} and optionally substituted alkyl are independently selected and R^{PR} are independently —H, a protecting group or an organic moiety as described for ester, alkyl or optionally substituted alkyl. Typically, urea groups as used here comprise an organic moiety containing about 1-50 carbon atoms, 1-20 carbon atoms or 1-8 carbon atoms and 0 to 10 independently selected heteroatoms (e.g., O, S, N, P, Si), typically 0-2, bonded through a suitable structure such as —NH—C(O)—NR^{PR}— structure, e.g., organic moiety-NH—C(O)—NR^{PR}—. Ureas also include urea moieties such as polypeptide-NH—C(O)—NR^{PR}—, and polymer-NH—C(O)—NR^{PR}—.

Spiro ring substituents as used here refers to cyclic structures that are usually 3, 4, 5, 6, 7 or 8 membered rings, e.g., they include 3, 4-, 5-, 6-, 7- or 8-sided rings. Spiro structures may also be defined by a cyclic ketal, thioketal, lactones or orthoesters.

As used herein, “monosaccharide” means a polyhydroxy aldehyde or ketone having the empirical formula (CH₂O)ₙ where n is 3, 4, 5, 6, 7 or 8. Typically, monosaccharides as used herein will contain 3, 4, 5, 6, 7 or 8 carbon atoms. Monosaccharide includes open chain and closed chain forms, but will usually be closed chain forms. Monosaccharide includes hexofuranose and pentofuranose sugars such as 2′-deoxyribose, ribose, arabinose, xylose, their 2′-deoxy and 3′-deoxy derivatives and their 2′,3′-dideoxy derivatives. Monosaccharide also includes the 2′,3′ dideoxydidehydro derivative of ribose. Monosaccharides include the D-, L- and DL-isomers of glucose, fructose, mannose, idose, galactose, allose, gulose, altrose, talose, fucose, erythrose, threose, lyxose, erythrulose, ribulose, xylulose, ribose, arabinose, xylose, psicose, sorbose, tagatose, glyceraldehyde, dihydroxyacetone and their monodeoxy or other derivatives such as rhamnose and glucuronic acid or a salt of glucuronic acid. Monosaccharides are optionally protected or partially protected. Exemplary monosaccharides include

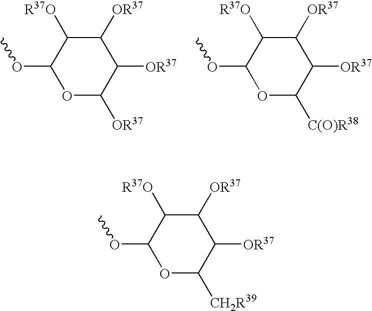

where R³⁷ independently is hydrogen, a protecting group, acetamido (—NH-Ac), optionally substituted alkyl such as methyl or ethyl, or an ester such as acetate or proprionate, R³⁸ is hydrogen, hydroxyl, —NH₂, —NHR^{PR}, optionally substituted alkyl such as methyl or ethyl, or a cation such as NH₄⁺, Na⁺ or K⁺ and R³⁹ is hydrogen, hydroxyl, acetate, proprionate, optionally substituted alkyl such as methyl, ethyl, methoxy or ethoxy.

Optionally substituted “monosaccharide” comprise any C₃-C₇ sugar, D-, L- or DL-configurations, e.g., erythrose, glycerol, ribose, deoxyribose, arabinose, glucose, mannose, galactose, fucose, mannose, glucosamine, N-acetylneuraminic acid, N-acetylglucosamine, N-acetylgalactosamine that is optionally substituted at one or more hydroxyl groups or hydrogen or carbon atoms. Suitable substitutions are as described above for substituted alkyl moieties and include independently selected hydrogen, hydroxyl, protected hydroxyl, carboxyl, azido, cyano, —O—C₁₋₆ alkyl, —S—C₁₋₆ alkyl, —O—C₂₋₆ alkenyl, —S—C₂₋₆ alkenyl, ester, e.g., acetate or proprionate, optionally protected amine, optionally protected carboxyl, halogen, thiol or protected thiol.

Optionally substituted “oligosaccharide” comprises two, three, four or more of any C3-C7 sugars that are covalently linked to each other. The linked sugars may have D-, L- or DL-configurations. Suitable sugars and substitutions are as described for monosaccharides. The linkage between the monosaccharides that comprise the oligosaccharide is α or β. Adjacent monosaccharides may be linked by, e.g., 1→2, 1→3, 1→4, and/or 1→6 glycosidic bonds. Oligosaccharide also includes oligosaccharide moieties typically found on the Fc region of an antibody such as an IgG antibody. In some embodiments an antibody is immobilized onto a biopolymer material through its carbohydrate moieties as described elsewhere in the specification to provide an optical sensor

As used herein, “polymer” is a molecule formed by the joining of smaller monomer units in a regular pattern or is a molecule having a repeating arrangement of one or more types of monomer units. Polymers includes biocompatible synthetic organic polymers, e.g., polyethyleneglycols (“PEGs”), polypropyleneglycol ethers, poloxalenes, polyhydroxyalkyl polymers, poloxamers or ethoxylated/propoxylated block polymer. PEG means an ethylene glycol polymer that contains 2-50 or more linked ethylene glycol monomers. Average PEG molecular weights can be about 80, 100, 200, 300, 400, 500, 600, 1000, 1200, 1500, 2000, 8000, 10,000, 20,000 or 30,000 and mixtures thereof are included, e.g., PEG100 and PEG200, PEG200 and PEG300, PEG100 and PEG300, PEG100 and PEG400 or PEG200 and PEG400. PEG polymers include methyl or alkyl ethers such as H(OCH₂HC₂)ₙ—OH, H(OCH₂HC₂)ₙ—CH₃, H(OCH₂HC₂)ₙ—OR^{PR} and analogs containing thiol, amine, azido (as amine surrogate) and carboxylic acids groups and their protected derivatives such as CH₃(OCH₂HC₂)ₙ—SH, CH₃(OCH₂HC₂)ₙ—S—S—(CH₂CH₂O)ₙ—CH₃, H(OCH₂HC₂)ₙ—N₃, H(OCH₂HC₂)ₙ—COOR^{PR}. PEG polymers also include homo- and hetero-bifunctional PEG derivatives having thiol, amine and carboxylic acid functional groups and their protected derivatives such as HOOC—CH₂CH₂—(OCH₂CH₂)ₙ—S—S—CH₂CH₂COOH, H(OCH₂HC₂)ₙ—OCH₂CH₂COOR^{PR}, HOOC—CH₂CH₂—(OCH₂CH₂)ₙO—CH₂CH₂COOH, NH₂—CH₂CH₂—(OCH₂CH₂)ₙ—NHR^{PR}, HS—(CH₂CH₂O)ₙ—COOH, HOOC—CH₂CH₂—(OCH₂CH₂)ₙ—OCH₂CH₂NHR^{PR}, where R^{PR} is a protecting group and n or the average value of n is about 2, 3, 4, 5, 6, 7, 8, 9, 10, 11, 12, 13, 14, 15, 20, 25, 30, 35, 40, 45 or 50. Lower molecular weight PEG polymers containing up to 28 monomer units may be obtained in monodispersed form. Various monodispersed, homo and heterobifunctional PEG polymers may be obtained from CreativeBiochem, Winston Salem, N.C. Preparation of heterobifunctional PEG polymers and their use in attaching to proteins is disclosed in US 20070238656 (Harder, et al.), US 20050176896 (Bentley) and U.S. Pat. No. 7,217,845 (Rosen). Various heterobifunctional PEG are disclosed elsewhere in the specification, particularly in Table 4.

Poloxamers typically have average molecular weights of one, two or more of about 1000, 2000, 4000, 5000, 6000, 8000, 10,000, 12,000, 14,000, 15,000 and/or 16,000, with structures such as HO(CH₂CH₂O)ₐ—(CH(CH₃)CH₂OH)_{b}—(CH₂CH₂O)_{c}—H, R^{PR}HN—(CH₂CH₂O)ₐ—(CH(CH₃)CH₂OH)_{b}—(CH₂CH₂O)_{c}—H, HS(CH₂CH₂O)ₐ—(CH(CH₃)CH₂OH)_{b}—(CH₂CH₂O)_{c}—H or R^{PR}O(CH₂CH₂O)ₐ—(CH(CH₃)CH₂OH)_{b}—(CH₂CH₂O)_{c}—H, where R^{PR} is a protecting group and n or the average value of b is at least about 15 or 20 and a+c varies from about 20% to about 90% by weight of the molecule, e.g., a and/or c is about 5, 7, 10, 15, 20, 25, 30, 35, 40, 45, 50, 55, 60, 65, 70, 75 and/or 80. Exemplary poloxamers include pluronic L62LF where a is about 7, b is about 30 and c is about 7, pluronic F68 where a is about 75, b is about 30 and c is about 75 and pluronic L101 where a is about 7, b is about 54 and c is about 7. Exemplary poloxalenes include structures such as HO(CH₂CH₂O)ₐ—(CH(CH₃)CH₂OH)_{b}—(CH₂CH₂O)_{c}—H or R^{PR}O(CH₂CH₂O)ₐ— (CH(CH₃)CH₂OH)_{b}—(CH₂CH₂O)_{c}—H, where R^{PR} is a protecting group and the average value for a is about 12, b is about 34 and c is about 12 or the average molecular weight is about 3000. Polymers also include derivatives of any of these molecules where one or both terminal hydroxyl groups and/or one, two, three or more internal hydroxyl groups are derivatized, e.g., to independently selected moieties such as —C(O)—OR^{PR}—C(O)—OH, —C(S)—OH, —SH, —SR^{PR}, —C(O)—SH, —C(O)—SR^{PR}, —NH₂, —NHR^{PR}, —N(R^{PR})₂, —C(O)NH₂, —C(O)NHR^{PR}, —C(O)N(R^{PR})₂ or a salt, where R^{PR} independently or together are a protecting group or C1-C8 optionally substituted alkyl.

“Biopolymer” as used here is a type of polymer comprised of monomer units found in polymers of biological origin. Examples of biopolymers are found in polysaccharide, nucleic acid polymers (e.g. DNA, RNA), and polypeptides, which are comprised of monosaccharide, nucleic acid and amino acid monomer units, respectively. Biopolymer also includes lipids, which in biological origin have methylene (—CH₂—) as a repeating monomer unit group and may contain one or more alkenyl moieties (i.e., —C═C—). A biopolymer may be derived from a biological source or prepared synthetically. Synthetic biopolymers may be comprised or consist of monomer units that are natural or un-natural or a combination of both. Thus, a polypeptide biopolymer may contain natural or un-natural amino acids as is described elsewhere in the specification for polypeptide, and a polysaccharide may contain natural or unnatural monosaccharides or a combination of both, which are described elsewhere in the specification for monosaccharide. Similarly a synthetic lipid biopolymer has methylene (—CH₂—) as a repeating monomer unit group and may contain one or more alkenyl moieties (i.e., —C═C—) or alkynyl moieties or other unsaturated carbon-based moieties. In one embodiment a biopolymer contains methylene monomer units and at least one unsaturated carbon based moiety that is capable of cross-linking to another unsaturated carbon-based moiety located in a physically adjacent biopolymer. The unsaturated carbon based moiety capable of crosslinking to another such moiety is referred to as a polymerization unit

A lipid biopolymer will often contain a functional group as a head group. Examples of such biopolymer functional head groups are, by way of example and not limitation, a carboxylic acid, hydroxyl, amino, sulfhydryl, ketone or aldehyde group, either free or in protected form. Sometimes a lipid biopolymer will contain a surrogate head group which can be transformed synthetically or enzymatically to one of the biopolymer functional head groups given above after assembly of the lipid biopolymers to provide a biopolymer material.

Typically, a synthetic lipid biopolymer comprises a head group, 2-50 methylene monomer units and a polymerization unit which allows for crosslinking of the lipid polymers to provide a biopolymer material. Sometimes the polymerization unit is comprised or consists of two adjacent alkynyl moieties, referred to as a di-acetylene moiety (i.e., —CC—CC—) and resides in a polymer chain having from 15-25 or 20-30 carbon atoms in length. A biopolymer containing a di-acetylenic moiety as the polymerization unit is referred to as a DA-monomer. In one embodiment a DA-monomer has a di-acetylene moiety and a lipid head group wherein the di-acetylene moiety is positioned in the polymer chain of the DA monomer from between positions 18-20 from the lipid head group to positions 3-5. In another embodiment the di-acetylene moiety is between positions 10-12 to positions 4-6. In yet another embodiment the di-acetylene groups are positioned at about the 5-7 position. Example DA-monomers with a carboxylic acid head group include, but are not limited to, 5,7-docosadiynoic acid (5,7-DCDA), 5,7-pentacosadiyonic acid (5,7-PCA) or 10,12-pentacosadiynoic acid (10,12-PCA). Variation in sensitivity (i.e. the maximal change in an optical property observable for a biopolymer material subsequent to binding of a biomarker to a biomarker receptor immobilized thereto) are sometimes observed that are dependent on the position of the polymerization unit within the carbon chain of the lipid biopolymer. It will be within the ability of the skilled artisan to vary the position of a polymerization unit such as a di-acetylene moiety, to achieve maximum sensitivity for an optical sensor based upon such polymerization units.

“Biopolymer material” refers to materials composed of polymerized biopolymers. A biopolymer material may have a physical form including, but not limited to, films, vesicles, liposomes, tubules, braided assemblies, lamellar assemblies, helical assemblies, multilayers, aggregates, membranes, and solvated polymer aggregates such as rods and coils in solvent. A Biopolymer material can further contain molecules that are not part of the matrix of the polymerized biopolymer (i.e., molecules that are not polymerized). In one embodiment, the biopolymer material is in the form of vesicles or liposomes. In another embodiment, the biopolymer material is in the form of a film. Films include monolayers, bilayers, and multilayers. Monolayers and films, in this context, are solid state materials that are supported by an underlying substrate (i.e., a biopolymer support). Such monolayers and films have been reviewed in Ulman (1991) and Gaines (1966) among others. In contrast to films and monolayers, liposomes are three-dimensional vesicles that enclose an aqueous space. These materials are described in New (1989) and Rosoff (1996) among others. Liposomes can be constructed so that they entrap materials within their aqueous compartments. Films and monolayers do not enclose an aqueous space.

In one embodiment a biopolymeric film is prepared by layering self-assembling organic monomers over a support. In some embodiments the support is a standard Langmuir-Blodgett trough and the self-assembling organic monomers are biopolymers that are layered onto an aqueous surface created by filling the trough with an aqueous solution. The biopolymer is then compressed and polymerized to form a biopolymer film. Films so produced are referred to as Langmuir-Blodgett films. In one embodiment the compression is conducted in a standard Langmuir-Blodgett trough using moveable barriers to compress the biopolymers. Compression is carried out until a tight-packed layer of the biopolymers are formed which are then polymerized. In some embodiments, lipid biopolymers, comprised of di-acetylene moieties (i.e., a di-acetylene monomer), are used as the self-assembling monomer. The di-acetylene monomers are polymerized to give a polydiacetylene (PDA) biopolymer film using ultraviolet irradiation. In some embodiments a Langmuir-Blodgett film (LB film) prepared from a lipid biopolymer monomer, such as a DA-monomer, is transferred to a hydrophobized biopolymer support, which is described elsewhere in the specification, such that the lipid head groups are exposed at the film-ambient interface (Charych 1993).

A biopolymer material may be prepared from polymerization of one or more different biopolymer monomers that have a common polymerization unit. Oftentimes two or more biopolymers, typically two, will have different head groups wherein at least one head group will provide for a reactive functional group (or a surrogate thereof) that will permit covalent attachment of a biomarker receptor, and will be mixed in a proportion to provide a desired density of each head group. The density of the head group that provides the reactive functional group, either directly or after further chemical manipulations (i.e. a surrogate of the reactive functional group) and will be an important consideration and dependent on the size of the biomarker receptor to be immobilized, the type of head group, and the physical form of the biopolymer material. For example, in preparation of a Langmuir-Blodgett film (LB film) incorporating DA-monomers, the maximum proportion of a DA-monomer having a head group that is charged under conditions required for formation of the film, such as a carboxylic acid head group, that may be used will be lower than if a DA-monomer is used having a non-charged, hydrophilic head group, such as an ester, due to electrostatic repulsion. Furthermore, for immobilization of more sterically demanding biomarker receptors to a PDA-biopolymer film, such as a biomarker receptor comprised of multiple polypeptides, a lower density of biomarker receptors will be achievable than if a biomarker receptor having a single polypeptide is used and will thus serve as a guide to the relative amount of the DA-monomer having the reactive functional group (or a surrogate thereof) to be used.

An alternative to immobilization of a biomarker receptor after formation of a PDA-biopolymer material is to use a DA-monomer in which the biomarker receptor is already attached. Another alternative is to attach a biomarker receptor to a hydrophobic polymer than can self-assemble with a collection of DA-monomers that when polymerized to form a LB film results in entrainment of the biomarker receptor-bearing polymer. In essence, the biomarker receptor-bearing polymer serves as a dopant in the formation of the LB film resulting in a non-covalent attachment (i.e., immobilization) of a biomarker receptor to a biopolymer material. As a general guideline preparations of optimal biopolymer materials will typically have 5-15% of the theoretical maximum density of biomarker receptors either from their incorporation into DA-monomers or as a biomarker receptor-bearing dopant incorporated into a collection of biopolymers to be polymerized into a biopolymer material.

Liposomes are prepared by dispersal of amphiphilic molecules in an aqueous media and remain in the liquid phase. Liposomes exist within homogenous aqueous suspensions and may be created in a variety of shapes such as spheres, ellipsoids, squares, rectangles, and tubules. Thus, the surface of a liposome is in contact with liquid only—primarily water. In some respects, liposomes resemble the three-dimensional architecture of natural cell membranes.

Methods to prepare Langmuir-Blodgett films, liposomes and sol-gels from DA-monomers are given in US patent application 2003/0129618 (Moronne), and U.S. Pat. Nos. 6,395,561, 6,468,759, 6,485,987, 6,180,135, 6,183,772, 6,103,217, 6,080,423, 6,001,556 and 6,022,748.

“Biopolymer substrate” or “biopolymer support” as used herein refers to a solid object or surface upon which a biopolymer material or optical sensor is immobilized and may be comprised of a rigid or flexible material. Biopolymer supports include plastics (e.g., polystyrene or polyethylene, mica, filter paper (e.g., nylon, cellulose, and nitrocellulose), glass beads and slides, gold and all separation media such as silica gel or sephadex, and other chromatographic media. In some embodiments, a biopolymer material is immobilized in silica glass using the sol-gel process. In one embodiment the biopolymer support is rigid or resists deformation to a greater extent than a biopolymer material or an optical sensor to be immobilized onto the biopolymer support. Sometimes an inert material to be used as a biopolymer support is chemically treated to provide a biopolymer support having hydrophobic groups (i.e., hydrophobized) that will immobilize a biopolymer material through hydrophobic interactions. A material so chemically treated is referred to as hydrophobized biopolymer support.

In some embodiments, a biopolymer support is composed of a glass, quartz or a plastic or any other material having a thickness that provides the necessary resistance to deformation and remains capable of permitting the detection of optical energy emitted by an optical sensor which is comprised of the biopolymer substrate and the biopolymer material. Thus, a biopolymer support onto which a biopolymer material is immobilized will provide physical support for the biopolymer material and be transparent to a first wavelength range that encompasses the wavelength of optical energy to be emitted by a biopolymer material upon incorporation of the biopolymer material to provide an optical sensor. Typically, a biopolymer material will be immobilized to a surface of a biopolymer support, referred to as a biopolymer support surface, at a surface of the biopolymer material, referred to as a biopolymer material surface, that will minimize interference with binding of a biomarker to a biomarker receptor that is or will be immobilized to the biopolymer material. In one embodiment the biopolymer material is a Poly-di-acetylenic (PDA) biopolymer film that is immobilized to a biopolymer substrate surface from the opposing surface of the PDA-biopolymer film on which the biomarker receptor is or will be immobilized. In another embodiment a biopolymer substrate is a glass slide wherein the glass slide includes but not limited to a quartz or borosilicate glass slide. To immobilize a biopolymer material to glass or quartz support a surface of the support is often derivitized with a silylating agent having a reactive functional group that is subsequently used to covalently bind a hydrophobic molecule. In one embodiment a silylating agent used to derivatize a glass support has an amino group (i.e., an aminosilyating agent) as the reactive functional group. In one embodiment the aminosilyating agent is 3-aminopropyltriethoxy silane. Silyation of glass with aminosilylating agents and silylating agents having other reactive functional groups is described in U.S. Pat. No. 4,024,235 (Weetall) and U.S. Pat. No. 3,519,538 (Messing) which are incorporated by reference herein.

A primary function of the biopolymer support is to provide physical support for the biopolymer material. The biopolymer support may also serve a secondary purpose whereby an optical property to be produced by a biopolymer material or optical material so supported is manipulated by the biopolymer material. The secondary purpose of manipulation of an optical property includes but is not limited to focusing, redirecting, filtering or amplifying.

In one embodiment a biopolymer support for a biopolymer or an optical sensor is a glass vial including but not limited to a quartz or borosilicate glass vial. In another embodiment the biopolymer support for a biopolymer material or an optical sensor comprises the inside bottom surface of a microtiter plate well or inside surface of a wall of a cuvette wherein the cuvette wall is capable of receiving incident light and permitting detection of a detectable change in an optical property of the biopolymer material supported by the cuvette. Microtiter plates to provide support for a biopolymer material or an optical sensor film include but are not limited to 96, 384, 1536 well plates having square or rounded well sides and flat, V-shape or rounded bottoms. Lower density microtiter plates such as 24 or 48 well plates may also be used to provide increased sensitivity, but requiring a greater volume of a fluid of biological origin suspected of having a biomarker to be detected. Microtiter plates preferred as supports for optical sensors intended for use in an automated biosensor device will conform to ANSI-SBS standards. Microtiter plates suitable for use as supports for optical sensors have wells composed of plastic, including but not limited to polyvinyl, polypropylene, polystyrene, or are composed of borosilicate glass or quartz or have bottom surfaces composed of plastic, borosilicate glass or quartz. Appropriate choice of microtiter plate format will depend on requirements of sensitivity, throughput, sample volume and optical property to be detected and are familiar parameter to be optimized to one of ordinary skill in the art. For example, a microtiter plate with flat bottom wells provides low background absorbance, a microtiter plate with round bottom wells provides enhance sensitivity in fluorescence applications and microtiter plates with plastic wells are useful for immobilization of a hydrophobic biopolymer material having an optical property to be measured at a wavelength within the visible light spectrum. Other biopolymer substrates that provide a surface for supporting a biopolymer material further include an absorbance flow cell or a fluorescence flow cell.

“Polypeptide” as used herein refers to a single polypeptide or a complex of two, three, four, or more polypeptides having the same or different amino acid sequences (e.g., tumor necrosis factor (TNF) is a complex of three identical polypeptides, a homotrimer). A cellular receptor or ligand often is a native polypeptide, which then is modified and immobilized onto a biopolymer material to comprise an optical sensor and then becomes a biomarker receptor. The cellular receptor or ligand sometimes is characterized as having a molecular weight between about 10 kDa and about 50 kDa, and sometimes between about 15 kDa to about 35 kDa. The cellular receptor or ligand sometimes is a fragment of a native polypeptide, where the fragment at times is a domain of the polypeptide or a portion thereof. A polypeptide fragment is a portion of the polypeptide that and performs a function of the polypeptide (e.g., a cellular receptor domain that binds a ligand or ligand fragment, or a ligand domain that binds a cellular receptor or cellular receptor fragment). A polypeptide fragment can comprise a non-domain region of a native polypeptide and part of a domain, and sometimes is between about 20 and about 200 amino acids in length, and often between about 50 and about 100 amino acids in length. The cellular receptor or ligand sometimes is a polypeptide mimetic, which includes non-native amino acids and/or non-amino acid moieties, examples of which are known in the art and described hereafter.

For polypeptide molecules, an amino acid sequence or single amino acid may be deleted, inserted, or substituted using standard molecular biology techniques or peptide synthetic techniques. Methods for preparing amide bonds in polypeptides are additionally provided in the definition of amide. Any amino acid in a cellular receptor, a biomarker receptor, ligand or biomarker may be substituted or deleted or an insert may be introduced at any position, and a substitution may be by one of the other nineteen naturally occurring amino acids or a non-classical or unnatural amino acid. Amino acid substitutions may be made on the basis of similarity in polarity, charge, solubility, hydrophobicity, hydrophilicity, and/or the amphipathic nature of the residues as long as an attenuated function of the resulting polypeptide or peptide is retained. For example, negatively charged amino acids include aspartic acid and glutamic acid; positively charged amino acids include lysine and arginine; and amino acids with uncharged polar head groups having intermediate hydrophilicity values include leucine, isoleucine, valine, glycine, alanine, asparagine, glutamine, serine, threonine, phenylalanine, and tyrosine. Amino acid modifications often are performed using standard methods (e.g., Current Protocols In Molecular Biology Ausubel, F. M., et al., eds. (2000) and Sambrook et al., “Molecular Cloning: A Laboratory Manual,” 2nd ed. (1989)).

Conservative substitutions may be made, for example, according to Table A. Amino acids in the same block in the second column and in the same line in the third column may be substituted for one another other in a conservative substitution. Conservative substitutions sometimes are performed by replacing an amino acid in one row of the third column corresponding to a block in the second column with an amino acid from another row of the third column within the same block in the second column.

In certain embodiments homologous substitution may occur, which is a substitution or replacement of like amino acids, such as basic for basic, acidic for acidic, polar for polar amino acids, for example. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Amino acid substitutions sometimes are selected to enhance the hydrophobicity of the variant peptide, the amphipathic nature of a variant peptide, and to enhance or decrease the probability that a variant peptide forms an alpha-helical structure or substructure.

TABLE A
Conservative amino acid replacements within a polypeptide

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

A cellular receptor or ligand variant polypeptide sequence often is substantially identical to a native ligand or cellular receptor polypeptide sequence. The amino acid sequence of the variant at times is 50% or more, 51% or more . . . 60% or more, 61% or more . . . 70% or more, 71% or more . . . 80% or more, 81% or more . . . 85% or more . . . 89% or more, 90% or more, 91% or more, 92% or more . . . 95% or more . . . 97% or more, 98% or more, or 99% or more identical to a native ligand or receptor amino acid sequence.

Naturally occurring amino acids in a native ligand or cellular receptor protein polypeptide or protein sometimes are substituted with unnatural or non-classical amino acids, which include, but are not limited to, ornithine (hereinafter referred to as Z), diaminobutyric acid (hereinafter referred to as B), norleucine (hereinafter referred to as O), pyrylalanine, thienylalanine, naphthylalanine and phenylglycine. Other examples of non-naturally occurring amino acids and non-classical amino acid replacements are alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine, L-allyl-glycine*, beta-alanine*, L-alpha-amino butyric acid*, L-gamma-amino butyric acid*, L-alpha-amino isobutyric acid*, L-epsilon-amino caproic acid#, 7-amino heptanoic acid*, L-methionine sulfone*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid, L-Phe (4-benzyl)*, 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, fluoroamino acids, designer amino acids such as beta-methyl amino acids, Ca-methyl amino acids, Na-methyl amino acids, naphthyl alanine, and the like. The notation * indicates a derivative having hydrophobic characteristics, # indicates a derivative having hydrophilic characteristics, and #* indicates a derivative having amphipathic characteristics.

Variant amino acid sequences sometimes include suitable spacer groups inserted between any two amino acid residues of the sequence including alkyl groups such as methyl, ethyl or propyl groups in addition to amino acid spacers such as glycine or β-alanine residues. Also, peptides and polypeptides may comprise or consist of peptoids. The term “peptoids” refers to variant amino acid structures where the α-carbon substituent group is on the backbone nitrogen atom rather than the α-carbon. Processes for preparing peptides in the peptoid form are known in the art (see, e.g., Simon et al., PNAS 89(20): 9367-9371 (1992) and Horwell, Trends Biotechnol. 13(4): 132-134 (1995)).

Polypeptides and variants thereof, of which a cellular receptor, biomarker receptor, ligand or biomarker comprises of or consists of, often are prepared by known recombinant molecular biology procedures (e.g., Mullis et al., Methods Enzymol. 155:335-50 (1987) and Ausubel et al., Current Protocols in Molecular Biology, for example pages 3.17.1-10). Alternatively, the polypeptide is sometimes synthesized using chemical synthesis processes or in certain embodiments is purified from a biological source. Some purified enzymes, are commercially available.

A polypeptide may be synthesized by peptide ligation methods (see, e.g., Dawson et al., Science 266:776-9 (1994) and Coligan et al., Native chemical ligation of polypeptides, Wiley: 18.4.1-21 (2000)). This method allows native backbone proteins to be assembled from fully unprotected polypeptide building blocks. To facilitate the ligation reactions, the alpha-carboxylate group of the N-terminal polypeptide fragment is mildly activated as an aryl thioester and the C-terminal polypeptide fragment contains an amino-terminal cysteine. The reaction often is carried out in aqueous buffer at about neutral pH. The initial step is a reversible transthioesterification reaction involving the thiol group of the N-terminal Cys-polypeptide (the C-terminal fragment) and the alpha-thioester moiety of the N-terminal polypeptide fragment. This intermediate undergoes a spontaneous rearrangement to form a natural peptide bond at the ligation site. An advantage of the chemical approach is the site-specific incorporation of unnatural amino acids and post-translational modifications into the target molecule. Polypeptide fragments of about 50 amino acids or less, and mimetics and variants thereof, sometimes are produced by standard chemical synthetic methods known in the art (e.g., peptide synthesizer commercially available from Applied Biosystems).

Polypeptides and variants thereof, of which a cellular receptor, biomarker receptor, ligand or biomarker comprises of or consists of, are isolated using standard purification procedures. An “isolated” or “purified” peptide, polypeptide or protein is substantially free of cellular material or other contaminating proteins from the cell or tissue source from which the protein is derived, or substantially free from chemical precursors or other chemicals when chemically synthesized. “Substantially free” means preparation of a ligand, receptor, or peptide, polypeptide or protein variant thereof having less than about 30%, 20%, 10% and more preferably 5% (by dry weight), of non-receptor or ligand polypeptides (also referred to herein as a “contaminating proteins”), or of chemical precursors or non-receptor or ligand chemicals. When the polypeptide or a biologically active portion thereof is produced recombinantly, it often is substantially free of culture medium, specifically, where culture medium represents less than about 20%, sometimes less than about 10%, and often less than about 5% of the volume of the polypeptide preparation. Isolated or purified polypeptide preparations sometimes are 0.01 milligrams or more or 0.1 milligrams or more, and often 1.0 milligrams or more and 10 milligrams or more in dry weight.

“Receptor” as used herein means a molecule that interacts with a ligand by binding to the ligand with a K_{d} in a K_{d} range between 20×E-06 M to 1×E-15 M or lower and wherein the receptor transmits a biochemical or physiochemical signal upon binding of the ligand that is indicative of the receptor-ligand interaction. Receptors of biological origin or derivation are referred to as cellular receptors. A cellular receptor is to be immobilized onto a biopolymer to form an optical sensor is referred to as a cellular biomarker receptor and is an example of a biomarker receptor. A ligand of a cellular receptor that binds to a biomarker receptor is an example of a biomarker. In one embodiment the cellular biomarker receptor transmits a signal upon binging of the ligand of the cellular receptor to a biopolymer material to which the cellular receptor is immobilized. In another embodiment the K_{d} range is between 10×10E-06 M to 1×10E-12 M. In yet another embodiment the K_{d} range is between 1×10E-06 to 1×10E-10 or 0.1×10E-06 to 1×10E-9. In another embodiment the biomarker receptor is a cellular receptor fragment, wherein the cellular receptor fragment comprises or consists of the binding domain of the intact cellular receptor. Therefore, the cellular receptor fragment is a polypeptide that comprises the cellular receptor and performs a function of the cellular receptor. In one embodiment the cellular receptor fragment has a K_{d} for a biomarker in a K_{d} range 20×E-06 M to 1×E-15 M, 10×10E-06 M to 1×10E-12 M, 1×10E-06 to 1×10E-10 or 0.1×10E-06 to 1×10E-9.

The cellular receptor sometimes may be bound to a biological membrane in a cellular system (e.g. group of cells or a tissue of a mammal), bacterial or the envelope of a virus or in a disrupted membrane or envelope of a cell, bacteria or virus. In another embodiment the cellular receptor or cellular receptor fragment is unbound in a cell-free system or is immobilized to a biopolymer by a direct covalent attachment, an indirect covalent attachment or by direct or indirect non-covalent attachment, as defined herein, to a biopolymer to provide an optical sensor.

A cellular receptor sometimes is located in a membrane region within the membrane or at the membrane surface (e.g., receptor protein kinases, receptor protein phosphatases, cytokine receptors, G-protein coupled receptors and integrins. Sometimes the cellular receptor is located in the intracellular region of a cell and includes nuclear receptors. In one embodiment a cellular receptor or cellular receptor fragment is immobilized onto a biopolymer material by direct covalent attachment, indirect covalent attachment or a non-covalent attachment, as defined herein, to a biopolymer to provide an optical sensor. Thus, the cellular receptor or fragment thereof to be immobilized is referred to as a cellular biomarker receptor.

Cytokine receptors: Cytokine receptors are divided into four families. The Class I Cytokine Receptor Family includes cytokine-binding receptors that function in the immune and hematopoietic systems. In addition, this family includes receptors for growth hormone and prolactin. There are conserved amino acid sequence motifs in the extracellular domain with 4 positionally conserved cysteine residues (CCCC) and a conserved sequence of Trp-Ser-X-Trp-Ser (SEQ ID 1) where X is a non-conserved amino acid. The receptors consist of 2 polypeptide chains and are a cytokine-specific subunit and a signal-transducing subunit which is usually not specific for the cytokine ligand. In a few cases these receptors are trimers. The signal transducing subunit is required for high affinity binding of the cytokine ligand.

The Class I Cytokine Receptor Family is further divided into sub-families with all the receptors in one subfamily having an identical signal transducing subunit. The GM-CSF subfamily includes the receptors for IL-3, IL-5, and GM-CSF and is characterized by the low affinity, cytokine-specific receptor α subunit. All three low affinity α subunits associate noncovalently with a common signal transducing β subunit to form a dimmer receptor that exhibits increased affinity for the cytokine and transduces a signal across the membrane following cytokine binding. The IL-6 subfamily includes the receptors for IL-6, IL-11, and IL-12 characterized by a common signal transducing subunit (gp130) that associates with one or two different cytokine-specific subunits displaying overlapping biological activities. The IL-2 subfamily includes the receptors for IL-2, IL-4, IL-7, IL-9, and IL-15. The IL-2 and IL-15 receptors are trimers having a cytokine-specific a chain and two chains, β and γ that are responsible for signal transduction. The IL-2 receptor γ chain is the signal transducing subunit in those members of this subfamily which are dimers.

The Class II Cytokine Receptor Family, also known as the Interferon Receptor Family, include receptors for the three interferons, INF-α, β, and γ. These receptors possess the conserved cysteine motifs, but lack the WSXWS motif characteristic of the class I cytokine receptors.

The TNF Receptor Family includes the 55 kDa TNF receptor (TNF-RI) and the 75 kDa TNF receptor (TNF-RII), as well as CD40 and Fas. They have cysteine-rich repeats of about 40 amino acids in the extracellular amino terminal. Some members of the family display sequence similarities in their cytoplasmic regions as well. Both LT and TNF-α bind to the p55 receptor and the p75 receptor. The killing actions of TNF-α are mediated through the p55 receptor. The p55 receptor contains a conserved sequence motif called the “death domain” called TRADD that is involved in apoptosis. The p75 receptor contains a domain that defines a protein family of molecules called TNF-receptor associated factors (TRAFs). Their overexpression activates the transcription factor NFκB and also stress-activated protein kinase pathways that regulate transcription factor AP-1.

Structural homology partially distinguishes between cytokines that do not demonstrate a considerable degree of redundancy so that they can be classified into the following four types: The four α-helix bundle family whose member cytokines have three-dimensional structures with four bundles of α-helices. This family in turn is divided into three sub-families: 1) the IL-2 subfamily, 2) the interferon (IFN) subfamily and 3) the IL-10 subfamily. The first of these three subfamilies contains several non-immunological cytokines including erythropoietin (EPO) and thrombopoietin (THPO). Alternatively, four α-helix bundle cytokines can be grouped into long chain and short chain cytokines. The IL-1 family primarily includes IL-1 and IL-18. The IL-17 family member cytokines have a specific effect in promoting proliferation of T-cells that cause cytotoxic effects. The fourth family members are the chemokines, which are, along with the chemokine receptors, discussed below. A different classification divides immunological cytokines into those that promote the proliferation and functioning of helper T-cells, type 1 (IFN-γ etc.) and type 2 (IL-4, IL-10, IL-13, TGF-β etc.), respectively.

Soluble cytokine receptors can be found in the blood and extracellular fluid. These soluble receptors result from enzymatic cleavage of the extracellular domain of cell-bound cytokine receptors. The released soluble fragments can bind cytokine molecules, thereby neutralizing their activity. The soluble IL-2 receptor (sIL-2R) is released following chronic T cell activation. The shed receptor can bind IL-2 and prevent its interaction with the membrane-bound IL-2R.

The Chemokine Receptor Family members are G protein-coupled receptors with the N-terminal portion of chemokine receptors key to determining ligand binding specificity. Chemokine receptors are structurally related and can be categorized into specific (bind only one known ligand—e.g., CXCR1/IL8RA and CXCR4/fusin/LESTR), shared (CXCR2/IL8RB, CXCR3, CCCR1-CCCR5), promiscuous (bind to many chemokine ligands of either CXC or CC types), and viral (shared receptors that have been transduced into viral genomes during evolution, e.g. herpes saimiri virus and cytomegalovirus).

Chemokines are a family of structurally related glycoproteins with potent leukocyte activation and/or chemotactic activity. They are 70 to 90 amino acids in length and approximately 8 to 10 kDa in molecular weight. Most of them fit into two subfamilies with four cysteine residues. These subfamilies are base on whether the two amino terminal cysteine residues are immediately adjacent or separated by one amino acid. The α chemokines, also known as CXC chemokines, contain a single amino acid between the first and second cysteine residues; β, or CC, chemokines have adjacent cysteine residues. Most CXC chemokines are chemoattractants for neutrophils whereas CC chemokines generally attract monocytes, lymphocytes, basophils, and eosinophils. There are also 2 other small sub-groups. The C group has one member (lymphotactin). It lacks one of the cysteines in the four-cysteine motif, but shares homology at its carboxyl terminus with the C—C chemokines. The fourth subgroup is the C—X3-C subgroup. The C—X3-C chemokine (fractalkine/neurotactin) has three amino acid residues between the first two cysteines. It is tethered directly to the cell membrane via a long mucin stalk and induces both adhesion and migration of leukocytes.

Examples of cytokines receptors, cytokine receptor ligands and structural information thereof, such as peptide sequences, is provided by “http://apresslp.gvpi.net/apcyto/lpext.dll?f=templates&fn=main-h.htm&2.0” maintained by Elsevier, B.V. the Netherlands and by COPE (Cytokines and Cells Online Pathfinder Encyclopedia) at “http://www.copewithcytokines.de/cope.cgi.” and in “The Cytokine Handbook” 2^{nd} ed., Thomson A. Ed. Academic Press 1994. In one embodiment a cytokine receptor or a cytokine is immobilized on to a biopolymer material to provide an optical sensor and the cytokine receptor or cytokine to be immobilized is referred to as a cytokine biomarker receptor. A cytokine or chemokine that binds to a cytokine biomarker receptor is referred to as a cytokine biomarker.

G Protein-coupled receptors: G protein-coupled receptors (GPCRs) are a superfamily of proteins having seven membrane spanning domains and include receptors for sensory signal mediators (e.g., light and olfactory stimulatory molecules); adenosine, bombesin, bradykinin, endothelin, y-aminobutyric acid (GABA), hepatocyte growth factor, melanocortins, neuropeptide Y, opioid peptides, opsins, somatostatin, tachykinins, vasoactive intestinal polypeptide family, and vasopressin; biogenic amines (e.g., dopamine, epinephrine and norepinephrine, histamine, glutamate (metabotropic effect), glucagon, acetylcholine (muscarinic effect), and serotonin); chemokines; lipid mediators of inflammation (e.g., prostaglandins and prostanoids, platelet activating factor, and leukotrienes); and peptide hormones (e.g., calcitonin, C5a anaphylatoxin, follicle stimulating hormone (FSH), gonadotropic-releasing hormone (GnRH), neurokinin, and thyrotropin releasing hormone (TRH), and oxytocin). GPCRs which act as receptors for stimuli that have yet to be identified are known as orphan receptors.

GPCRs are divided into five classes based on sequence homology and functional similarity. Class A (rhodopsin-like), Class B (secretin-like), Class C (metabotropic/pheromone), Class D (Fungal pheromone), Class E (cAMP receptors) and Class F (Frizzled/Smoothened). Class A is further subdivided into 19 subgroups (A1-A19). Further description of GPCR classification is given in Foord, et al. “International union of pharmacology XLVI: G protein-coupled receptor list” Pharm. Rev. 2005, 57:279:288 and Joost, Genome Biol. 2002, 3(11):research0063.1-0063.16.

Examples of GPCRs and structural information thereof, such as their peptide sequences, is provided by “http://www.gpcr.org/7tm/” maintained by the GPCRDB (G Protein-coupled database) consortium, “http://www.expasy.org/cgi-bin/lists?7tmrlist.txt” maintained by the Swiss Institute of Bioinfomatics and in “The G-Protein Linked Receptor Facts Book”, Watson, S; Arkinstall, S, Academic Press 1994, which is incorporated by reference herein. In one embodiment a GPCR within a biomembrane is immobilized by a direct covalent attachment, an indirect covalent attachment or a non-covalent attachment through the GPCR polypeptide or through the biomembrane within which the GPCR resides to a biopolymer material to provide an optical sensor. The GPCR to be so immobilized is referred to as a GPCR biomarker receptor. In another embodiment a polypeptide ligand or a fragment thereof which binds to a peptidergic G protein-coupled receptor is immobilized onto a biopolymer material to provide an optical sensor. The polypeptide derived from a peptidergic GPCR ligand to be so immobilized is now referred to as a peptidergic GPCR biomarker. Examples of GPCRs and structural information thereof, such as their peptide sequences, is provided by Geppetti, Ed. In “Peptidergic G protein-coupled receptors” NATO Science series. Series A: Life Sciences, Vol. 307, IOS Press, 1999.

Integrins: Integrins are a superfamily of cell surface proteins that are cell surface receptors that play a role in the attachment of cells to other cells and in the attachment of a cell to the material part of a tissue that is not part of any cell (the extracellular matrix). Integrins also play a role in signal transduction, a process by which a cell transforms one kind of signal or stimulus into another, and define cellular shape, mobility, and regulate the cell cycle. Integrins are integral membrane proteins that are attached to the cellular plasma membrane through a single transmembrane helix of about 40-70 amino acids. The exception is the β₄ subunit which has a cytoplasmic domain of 1088 amino acids Most Integrins are heterodimeric with a α subunit of 95 kD that is conserved through the superfamily, and a more variable β subunit of 150-170 kD.

Beta (β) subunits have four cysteine-rich repeated sequences and are directly involved in coordinating at least some of the ligands to which integrins bind while the α subunits may stabilize the folds of the protein. In addition, variants of some of the subunits are formed by differential splicing, for example 4 variants of the β₁ subunit exist. Integrins include the fibronectin and vitronectin receptors of fibroblasts, which bind to an RGD (Arg-Gly-Asp) sequence in the ligand protein, the platelet IIb/IIIa surface glycoprotein (fibronectin and fibrinogen receptor), the LFA-1 class of leucocyte surface protein and the VLA surface protein. The requirement for the RGD sequence in the ligand is not invariable.

The structure between the alpha subunits is very similar. All contain 7 homologous repeats of 30-40 amino acids in their extracellular domain, spaced by stretches of 20-30 amino acids. The three or four repeats are mostly extracellular and contain sequences with cation-binding properties. These sequences are thought to be involved in the binding of ligands, because the interaction of integrins with their ligand is cation-dependent. All the α subunits share the 5 amino acid motif GFFKR, which is located directly under the transmembrane region. The alpha subunits are subdivided into two groups based on some structural differences. The first group is formed by alpha-1, alpha-2, alpha-L, alpha-M and alpha-X. The members of the first group all contain a so-called inserted domain (I-domain). This domain of about 180 amino acids is situated between the second and the third repeat and may be involved in ligand binding. The second group is formed by alpha-3, alpha-5, alpha-6, alpha-7, alpha-8, alpha-IIb, alpha-V and alpha-IEL. Members of this group all share a post-translational cleavage of their precursor into a heavy and a light chain. The light chain is composed of the cytoplasmic domain, the transmembrane region and a part of the extracellular domain (about 25 kD), while the heavy chain contains the rest on the extracellular domain (about 120 kD).

In mammals, 19 alpha and 8 beta subunits have been characterized and are designated as follows. Alpha-subunit: ITGA1 (CD49a), ITGA2 (CD49b), ITGA2B (CD41), ITGA3 (CD49c), ITGA4 (CD49d), ITGA5 (CD49e), ITGA6 (CD49f), ITGA7, ITGA8, ITGA9, ITGA10, ITGA11, ITGAD (CD11d), ITGAE (CD103), ITGAL (CD11a), ITGAM (CD11b), ITGAV (CD51), ITGAW and ITGAX (CD11c) also referred to as α₁, α₂, α₃, α₄, α₅, α₆, α₇, α₈, α₉, α₁₀, α₁₁, α_{D}, α_{E}, α_{L}, α_{M}, α_{V}, α_{W} and α_{X}, respectively; Beta-subunit: ITGB1 (CD29), ITGB2 (CD18), ITGB3 (CD61), ITGB4 (CD104), ITGB5, ITGB6, ITGB7 and ITGB8 also referred to a β₁, β₂, β₃, β₄, β₅, β₆, β₇ and β₈. Through different combinations of these α and β subunits, some 24 unique integrins are generated and include α₁β₁, α₂β₁, α₄β₁ (VLA-4), α₅β₁, α_{L}β₂ (LFA-1), α_{M}β₂ (Mac-1), α_{IIb}β₃, α₆β₄.

Examples of integrins and structural information thereof, such as peptide sequences of integrins and ligand thereof, is provided by “The Integrin Page” located at “http://www.geocities.com/CapeCanaveral/9629/”. In one embodiment an integrin or a β subunit thereof is immobilized to a biopolymer material through a polypeptide of integrin or integrin subunit, or through a biomembrane within which resides the polypeptide, by a direct covalent, an indirect covalent or a non-covalent attachment. An integrin or subunit or fragment thereof to be immobilized to provide an optical sensor is referred to as an integrin biomarker receptor and the ligand or a fragment thereof which binds to the integrin is referred to as an integrin biomarker.

Nuclear receptors: Nuclear receptors (NRs) are a class of intracellular proteins that are responsible for sensing the presence of hormones and certain other molecules. In response to agonist (typically a hormone or a small lipophilic molecule) binding, a nuclear receptor binds directly to DNA at a site referred to as its nuclear receptor response element (NRE) and subsequently regulates the expression of adjacent genes. The known 48 known human nuclear receptors categorized according to sequence homology as is shown as Subfamily: name; Group: name (endogenous ligand if common to entire group); Member: name (abbreviation; NRNC Symbol, gene) (endogenous ligand)

Subfamily 1: Thyroid Hormone Receptor-like; Group A: Thyroid hormone receptor (Thyroid hormone); (1) Thyroid hormone receptor-α (TRα; NR1A1, THRA), (2) Thyroid hormone receptors (TRβ; NR1A2, THRB); Group B: Retinoic acid receptor (Vitamin A and related compounds) (1) Retinoic acid receptor-α (RARα; NR1B1, RARA), (2) Retinoic acid receptor-β (RARβ; NR1B2, RARB), (3) Retinoic acid receptor-γ (RARγ; NR1B3, RARG); Group C: Peroxisome proliferator-activated receptor (1) Peroxisome proliferator-activated receptor-α (PPARα; NR1C1, PPARA), (2) Peroxisome proliferator-activated receptor-β/δ (PPARβ/δ; NR1C2, PPARD), (3) Peroxisome proliferator-activated receptor-γ (PPARγ; NR1C3, PPARG); Group D: Rev-ErbA (1) Rev-ErbAα (Rev-ErbAα; NR1D1), (2) Rev-ErbAβ (Rev-ErbAβ; NR1D2): Group F: RAR-related orphan receptor (1) RAR-related orphan receptor-α (RORα; NR1F1, RORA) (2) RAR-related orphan receptor-β (RORβ; NR1F2, RORB), (3) RAR-related orphan receptor-γ (RORγ; NR1F3, RORC); Group H: Liver X receptor-like (3) Liver X receptor-α (LXRα; NR1H3), (2) Liver X receptor-β (LXRβ; NR1H2), (4) Farnesoid X receptor (FXR; NR1H4); Group I: Vitamin D receptor-like (1) Vitamin D receptor (VDR; NR1I1, VDR) (vitamin D), (2) Pregnane X receptor (PXR; NR1I2), (3) Constitutive androstane receptor (CAR; NR1I3)

Subfamily 2: Retinoid X Receptor-like; Group A: Hepatocyte nuclear factor-4 (HNF4) (1) Hepatocyte nuclear factor-4-α (HNF4α; NR2A1, HNF4A), (2) Hepatocyte nuclear factor-4-γ (HNF4γ; NR2A2, HNF4G); Group B: Retinoid X receptor (RXRα) (1) Retinoid X receptor-α (RXRα; NR2B1, RXRA), (2) Retinoid X receptor-β (RXRβ; NR2B2, RXRB), (3) Retinoid X receptor-γ (RXRγ; NR2B3, RXRG); Group C: Testicular receptor (1) Testicular receptor 2 (TR2; NR2C1), (2) Testicular receptor 4 (TR4; NR2C2); Group E (TLX/PNR) (1) Human homologue of the Drosophila tailless gene (TLX; NR2E1), (3) Photoreceptor cell-specific nuclear receptor (PNR; NR2E3); Group F: COUP/EAR (1) Chicken ovalbumin upstream promoter-transcription factor I (COUP-TFI; NR2F1), (2) Chicken ovalbumin upstream promoter-transcription factor II (COUP-TFII; NR2F2), (6) V-erbA-related (EAR-2; NR2F6).

Subfamily 3: Estrogen Receptor-like; Group A: Estrogen receptor (1) Estrogen receptor-α (ERα; NR3A1, ESR1), (2) Estrogen receptor-β (ERβ; NR3A2, ESR2), Group B: Estrogen related receptor (1) Estrogen related receptor-α (ERRα; NR3B1, ESRRA), (2) Estrogen related receptor-β (ERRβ; NR3B2, ESRRB), (3) Estrogen related receptor-γ (ERRγ; NR3B3, ESRRG); Group C: 3-Ketosteroid receptors (1) Glucocorticoid receptor (GR; NR3C1) (Cortisol), (2) Mineralocorticoid receptor (MR; NR3C2) (Aldosterone), (3) Progesterone receptor (PR; NR3C3, PGR) (4) Androgen receptor (AR; NR3C4, AR)

Subfamily 4: Nerve Growth Factor IB-like; Group A: NGFIB/NURR1/NOR1 (1) Nerve Growth factor IB (NGFIB; NR4A1), (2) Nuclear receptor related 1 (NURR1; NR4A2), (3) Neuron-derived orphan receptor 1 (NOR1; NR4A3). Subfamily 5: Steroidogenic Factor-like; Group A: SF1/LRH1 (1) Steroidogenic factor 1 (SF1; NR5A1), (2) Liver receptor homolog-1 (LRH-1; NR5A2). Subfamily 6: Germ Cell Nuclear Factor-like; Group A: GCNF) (1) Germ cell nuclear factor (GCNF; NR6A1). Subfamily 0: Miscellaneous Group B: DAX/SHP (1) DAX1, Dosage-sensitive sex reversal, adrenal hypoplasia critical region, on chromosome X, gene 1 (NR0B1), (2) Small heterodimer partner (SHP; NR0B2); Group C: Nuclear receptors with two DNA binding domains (2DBD-NR)).

Nuclear receptors are modular in structure and contain the following domains: (A-B) N-terminal regulatory domain: Contains the activation function 1 (AF-1) whose action is independent of the presence of ligand. The transcriptional activation of AF-1 is normally very weak, but it does synergize with AF-2 (see below) to produce a more robust upregulation of gene expression. The A-B domain is highly variable in sequence between various nuclear receptors. (C) DNA-binding domain (DBD): highly conserved and contains two zinc fingers which bind to specific sequences of DNA called hormone response elements (HRE). (D) Hinge region: flexible domain that connects the DBD with the LBD and influences intracellular trafficking and subcellular distribution. (E) Ligand binding domain (LBD): moderately conserved in sequence and highly conserved in structure between the various nuclear receptors. The structure of the LBD is referred to as an alpha helical sandwich fold in which three anti parallel alpha helices (the “sandwich filling”) are flanked by two alpha helices on one side and three on the other (the “bread”). The ligand binding cavity is within the interior of the LBD and just below three anti parallel alpha helical sandwich “filling”. Along with the DBD, the LBD contributes to the dimerization interface of the receptor and in addition, binds coactivator and corepressor proteins. The ligand binding domain contains the activation function 2 (AF-2) whose action is normally dependent on the presence of bound ligand. (F) C-terminal domain: Variable in sequence between various nuclear receptors.

A nuclear receptor or a domain or a fragment thereof that provides for an optical sensor is referred to as a nuclear biomarker receptor and the ligand of the nuclear receptor so immobilized become a biomarker referred and is referred to as a NR biomarker. A nuclear receptor fragment is a polypeptide that performs at least one of the binding functions of the native nuclear receptor.

Nuclear receptor nomenclature and classification are given in Zhang Z, et al “Genomic analysis of the nuclear receptor family: New insights into structure, regulation, and evolution from the rat genome”. Genome Res 2004, 14 (4):580-90; Nuclear Receptors Nomenclature Committee “A unified nomenclature system for the nuclear receptor superfamily” Cell 1999, 97 (2):161-3.

In one embodiment a androgen (AR) or estrogen (Era or ERb) receptor is immobilized onto a biopolymer material to provide a optical sensor to detect a biomarker having an androstene, androstane or estrogen steroid nucleus. In another embodiment a DNA sequence comprising or consisting of an ARE or ERE is immobilized onto a biopolymer material to provide an optical sensor to detect a biomarker comprised of an activated androgen or estrogen receptor.

Enzymes—“enzyme” as used herein is a polypeptide that catalyzes a chemical transformation of a molecule. A molecule so transformed by the enzyme is called an enzyme substrate whereas another molecule that prevents the transformation of the enzyme substrate is called an enzyme inhibitor and may itself also be an enzyme substrate (i.e. is turned over). An enzyme substrate that inhibits an enzyme by covalent modification of an amino acid residue of the enzyme active site is called a suicide inhibitor. In one embodiment the biomarker receptor is an enzyme or a fragment thereof, wherein the enzyme fragment comprises or consists of the catalytic domain of the enzyme. Therefore, the enzyme fragment is a polypeptide that comprises the enzyme and performs a function of the cellular receptor by binding the native enzyme substrate of the intact enzyme. The fragment does not need to catalyze the transformation of the enzyme substrate unless that activity is required for a change in an optical property of a biopolymer to which the enzyme fragment is immobilized. An enzyme or a fragment thereof to be immobilized to provide an optical sensor is referred to as an enzyme biomarker receptor and the substrate of the enzyme so immobilized is referred to as an enzyme biomarker.

Cellular receptors and cellular biomarker receptors also include enzymes and enzyme biomarker receptors. Examples of enzymes, by way of illustration and not limitation, include protein kinases, protein phosphatases, proteases, hydrolases, esterases and cholinesterases.

In one embodiment the biomarker receptor is an enzyme or fragment thereof wherein the enzyme or enzyme fragment is immobilized onto a biopolymer material by a direct covalent, indirect covalent or non-covalent attachment to provide an optical sensor. The enzyme or enzyme fragment to be immobilized is referred to as an enzyme biomarker receptor. A ligand or substrate of the enzyme so immobilized is referred to as an enzyme biomarker. In another embodiment, the enzyme biomarker is an enzyme substrate or an enzyme inhibitor. In yet another embodiment an enzyme that has been modified by a suicide inhibitor becomes a biomarker for a biomarker receptor.

In one embodiment the immobilized enzyme is a protein kinase or a protein phosphatase. An enzyme that adds a phosphate group to a polypeptide, which may be a polypeptide within the enzyme (auto catalysis) or a different polypeptide and provides a phospho-peptide is called a protein kinase. An enzyme that removes a phosphate group on a polypeptide is called a protein phosphatase. A protein kinase or protein phosphatase to be immobilized onto a biopolymer material to provide an optical sensor is referred to as a protein kinase or a phosphatase biomarker receptor. In one embodiment a biomarker is a phospho-peptide. In another embodiment a phospho-peptide is immobilized onto a biopolymer material to provide an optical sensor. The phospho-peptide so immobilized is now a biomarker receptor and is called a phospho-peptide biomarker receptor.

A biomarker receptor sometimes consists of or is comprised of a binding domain from a protein kinase or a protein phosphatase. In one embodiment the binding domain is derived from a tyrosine receptor kinase or a serine, threonine or tyrosine intracellular kinase. Examples of binding domains from protein kinases include SRC-homology 2 and 3 domains (SH2 and SH3 domains), Pleckstrin homology domains (PH domain), DbI homology domain (DH domain), common docking domain (CD domain) and RAS binding domain (RBD). By way of example and not limitation, a biomarker is (1) a phosphotyrosine-containing ligand that interacts with a biomarker receptor comprised of a SH2 domain (2) a polyproline (PP)-containing ligand that interacts with a biomarker receptor comprised of an SH3 domain (3) a phospholipid-containing ligand that interacts with a biomarker comprised of a PH domain or (4) a ligand containing an amino acid region from RAS that interacts with a biomarker receptor comprised of a RBD wherein the interaction results in a detectable optic change in a biopolymer material to which the SH2, SH3, PH domain or RAS region is immobilized.

In one embodiment a biomarker receptor is a polypeptide immobilized onto a biopolymer wherein the polypeptide is a substrate for a protein kinase. Addition of a phosphate group by the protein kinase to the immobilized polypeptide provides an immobilized phospho-peptide and results in a detectable change in an optical property of the biopolymer material. In another embodiment a detectable change in an optical property of a biopolymer material to which a phospho-peptide is immobilized that occurs after interaction of a SH2 domain from another polypeptide with the immobilized phospho-peptide.

Protein kinases include protein-serine/threonine specific protein kinases, protein-tyrosine specific kinases and dual-specificity kinases. Other protein kinases include protein-cysteine specific kinases, protein-histidine specific kinases, protein-lysine specific kinases, protein-aspartic acid specific kinases and protein-glutamic acid specific kinases

By way of example and not limitation protein kinases or binding domains thereof include AFK, Akt, AMP-PK, Aurora kinase, beta-ARK, Abl, ATM, Auro kinase, ATR, CAK, Cam-II, Cam-III, CCD, Cdc2, Cdc28-dep, CDK, Flt, Fms, Hck, CKI, CKII, Met, DnaK, DNA-PK, Ds-DNA, EGF-R, ERA, ERK, ERT, FAK, FES, FGR, FGF-R, Fyn, Gag-fps, GRK, GRK2, GRK5, GSK, H4-PK-1, IGF-R, IKK, INS-R, JAK, KDR, Kit, Lck, MAPK, MAPKKK, MAPKAP2, MEK, MEK, MFPK, MHCK, MLCK, p135tyk2, p37, p38, p70S6, p74Raf-1, PDGF-R, PD, PhK, PI3K, PKA, PKC, PKG, Raf, PhK, RS, SAPK, Src, Tie-2, m-TOR, TrkA, VEGF-R, YES, or ZAP-70. In particular embodiments, the kinase is Akt, Abl, CAK, Cdc2, Fms, Met, EGF-R, ERK1, ERK2, FAK, Fyn, IGF-R, Lck, p70S6, PDGF-R, PI3K, PKA, PKC, Raf, Src, Tie-2 or VEGF-R.

Protein kinase substrates, include, but are not limited to substrates for protein kinases such as Akt, Abl, CAK, Cdc2, Fms, Met, EGF-R, ERK1, ERK2, FAK, Fyn, IGF-R, Lck, p70S6, PDGF-R, PI3K, PKA, PKC, Raf, Src, Tie-2 and VEGF-R. In one embodiment a polypeptide comprising a protein kinase substrate is immobilized onto a biopolymer material to provide an optical biosensor and thus the polypeptide becomes a biomarker receptor. Information on protein kinases, protein kinase binding domains and nucleotide sequences encoding the same are found in Protein Kinase Resource located at “http://www.kinasenet.com” and maintained by the University of California, KinBase located at “http://www.kinase.com and maintained by the Salk Institute or by Woodgett, Ed. in “Protein Kinases”, IRL Press, 1994.

Antibody “Antibodies” as used herein are Y-shaped proteins and are sometimes produced by the immune system of a mammal for the purpose of preparing a biomarker receptor by using a biomarker as an antigen. The basic functional unit of an antibody so produced is an immunoglobulin (Ig) monomer comprised of two identical heavy chains and two identical light chains connected by disulfide bonds. Antibodies are additionally comprised of carbohydrates that are attached to some of the antibody amino acid residues.

There are five types of mammalian Ig heavy chain which defines the antibody isotype and are denoted by the Greek letters α, δ, ε, γ, and μ and are found in IgA, IgD, IgE, IgG, and IgM antibodies, respectively. In humans and mouse there a four γ-heavy chain subtypes, γ₁, γ₂, γ₃, γ₄ in humans and γ₁, γ₂ₐ, γ_{2b}, γ₃. The α and γ heavy chains contain approximately 450 amino acids, while μ and ε have approximately 550 amino acids. In humans there are two α-subtypes α₁ and α₂. The four IgG isotypes defined by the heavy chain subtypes provides the majority of antibody-based immunity. IgG is expressed on the surface of B cells and in a secreted form and has the highest affinity for the antigen that resulted in the immune response. IgM is present in the early stages of B cell mediated immunity before there is sufficient IgG.

Each heavy and light chain is composed of structural domains that contain about 70-110 amino acids and are classified into two different categories, variable (IgV), and constant (IgC). The constant domain (IgC) is identical in all antibodies of the same isotype, but differs in antibodies of different isotypes. The Ig domains possess a characteristic immunoglobulin fold in which two beta sheets create a “sandwich” shape held together by an intradomain disulfide bond formed between conserved cysteines and interactions with charged amino acids. Heavy chains γ, α and δ have a constant region composed of three tandem Ig domains, one variable (V_{H}) domain followed by a constant domain (CH1), a hinge region, and two more constant (CH2 and CH3) domains while heavy chains μ and ε have a constant region composed of four immunoglobulin domains. In mammals there are only two types of light chain, lambda (λ) and kappa (κ). A light chain has two tandem domains, one constant domain and one variable domain (V_{L}). The approximate length of a light chain is 211 to 217 amino acids. Each antibody contains two light chains that are identical, and in mammals, only one type of light chain, κ or λ is present in a given antibody.

Antibody structure may further be divided corresponding to the fragment produced from a particular protease digestion. Pepsin digestion cleaves the antibody within the hinge region that is C-terminal to the heavy intrachain disulfide linkages to form a F(ab′) fragment which contains two covalently attached F(ab′) fragments. Papain digestion cleaves the antibody within the hinge region that is N-terminal to the heavy interchain disulfide linkages to produce two identical Fab fragments and a Fc fragment. The Fab (fragment, antigen binding) contains the site that binds antigen (the paratope) and is composed of one constant and one variable domain from each heavy and light chain of the antibody. The paratope (antigen binding site) is located in amino terminal ends of the Fab fragment and is shaped by the variable domains from the heavy and light chains. The Fc (Fragment, crystallizable) region plays a role in modulating immune cell activity and is composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins an by so doing mediates different physiological effects including opsonization, cell lysis, and degranulation of mast cells, basophils and eosinophils.

The antibody isotype of a B cell changes during the cell's development and activation. Immature B cells, which have never been exposed to antigen, are known as naïve B cells and express only the IgM isotype in a cell surface bound form. B cells begin to express both IgM and IgD when they reach maturity and the co-expression of both these immunoglobulin isotypes renders the B cell ‘mature’ and ready to respond to antigen. B cell activation follows engagement of the cell bound antibody molecule, referred to as the B cell receptor (BCR), with an antigen, causing the cell to divide and differentiate into an antibody producing cell called a plasma cell. In this activated form the B cell starts to produce antibody in a secreted form rather than a membrane-bound form. Some daughter cells of the activated B cells undergo isotype switching, a mechanism that causes the production of antibodies to change from IgM or IgD to the other antibody isotypes, IgE, IgA or IgG.

Antibodies are generated in vivo by random combinations of a set of gene segments called somatic mutation that encode different antigen binding sites (or paratopes), followed by random mutations in this area of the antibody gene, which create further diversity. Somatic recombination of immunoglobulins, also known as V(D)J recombination, involves the generation of a unique immunoglobulin variable region. The variable region of each immunoglobulin heavy or light chain is encoded in several pieces—known as gene segments. These segments are called variable (V), diversity (D) and joining (J) segments. V, D and J segments are found in Ig heavy chains, but only V and J segments are found in Ig light chains. Multiple copies of the V, D and J gene segments exist, and are tandemly arranged in the genomes of mammals. In the bone marrow, each developing B cell will assemble an immunoglobulin variable region by randomly selecting and combining one V, one D and one J gene segment (or one V and one J segment in the light chain). As there are multiple copies of each type of gene segment, and different combinations of gene segments can be used to generate each immunoglobulin variable region, this process generates a huge number of antibodies, each with different paratopes, and thus different antigen specificities. Antibody genes also re-organize in a process called class switching that changes the base of the heavy chain to another, creating a different isotype of the antibody that retains the antigen specific variable region.

Class switching occurs in the heavy chain gene locus by a mechanism called class switch recombination (CSR). This mechanism relies on conserved nucleotide motifs, called switch (S) regions, found in DNA upstream of each constant region gene (except in the 6-chain). The DNA strand is broken by the activity of a series of enzymes at two selected S-regions. The variable domain exon is rejoined through a process called non-homologous end joining (NHEJ) to the desired constant region (γ, α or ε). This process results in an immunoglobulin gene that encodes an antibody of a different isotype.

In one embodiment the biomarker receptor is an antibody or an antibody fragment wherein the antibody or fragment thereof recognizes (i.e., binds to) an antigen or a biomarker. An antibody or an antibody fragment to be immobilized onto a biopolymer material to provide an optical sensor is referred to as an antibody biomarker receptor. A biomarker recognized by the antibody or a fragment thereof is referred to as an antibody biomarker. Sometimes the biomarker is an antigen. In one embodiment an antibody biomarker receptor recognizes a molecule produced in a mammal as a result of a disease state or is produced during the establishment or progression of the disease state in the mammal. In another embodiment the antibody biomarker receptor detects a molecule produced by a chemical or biological insult received by a mammal. In one embodiment, the molecule recognized is a polypeptide that has been modified by a chemical or biological agent that is or is suspected to be responsible for the chemical or biological insult. In another embodiment the molecule recognized is a polypeptide within a mammal (i.e., in vivo) that has been modified by the action of a chemical or biological agent or an environmental toxin. In yet another embodiment, an antibody biomarker receptor recognizes an polypeptide that resides outside the body of a mammal (i.e., in vitro) wherein the polypeptide is the same or different to a polypeptide that is or is suspected to be modified by exposure of a mammal to a chemical agent, such as a synthetic compound, an environmental toxin or a organophosphoryl pesticide and acts as a surrogate (i.e, serves as a model) for exposure to said agent. By way of illustration and not limitation the chemical agent or environmental toxin in each embodiment described above includes an organophosphate compound such as a pesticide or insecticide.

“Hypervariable domain” as used herein refers to the complimentarity determining regions (CDR's) and are the regions of the immunoglobulin molecule that contain most of the residues involved in the antibody binding site. The CDRs contain the hypervariable loops of an antibody that are located in the V_{H} and V_{L} domains. The three variable loops in V_{H} are called H1, H2, and H3, while those in V_{L} are L1, L2, and L3. Procedure for locating the CDRs of an antibody are give by Schlessinger, “Epitome: database of structure inferred antigenic epitopes” Nucl. Acid Res. 2006, 34:D777-7890 which is incorporated by reference herein.

“Antibody fragment” as used herein refers to a fragment of an immunoglobulin that comprises one or more variable domains of an intact antibody such that the fragment is able to recognize the same antigen that the intact antibody is able to recognize. Antibody fragments include, by way of example and not limitation, Fab, F(ab′)₂, Fab′, Fv, and scFv immunoglobulin fragments, which are further described in subsequent paragraphs. Thus, the term “antibody fragment” encompasses the aforementioned immunoglobulin fragments and other fragments having a hypervariable domain. The term “antibody” also encompasses the term “antibody fragment” unless explicitly indicated or indicated by context.

The different variable domains comprising an antibody fragment may be on the same or different polypeptide chain that were derived from the heavy and-or light chains of the intact antibody. The different polypeptide chains may be covalently attached by disulfide bond or held together entirely by non-covalent interactions. Thus, an antibody fragment includes Fab and F(ab′)₂ immunoglobulin fragments which may be derived from protease digestion of an antibody. The F(ab′)₂ may be reduced under mild conditions to break the disulfide linkage in the hinge region thereby converting the (Fab′)₂ dimer into a Fab′ monomer. The Fab′ monomer is essentially a Fab that contains part of the hinge region. While various antibody fragments are defined in terms of the digestion of an intact antibody, one of skill will appreciate that such fragments may be synthesized de novo either chemically or by utilizing recombinant DNA methodology.

Antibody fragments also include Fv fragments which contain only variable light (V_{L}) and variable heavy (V_{H}) domains and are in contrast with Fab fragments which contain the variable domains and part of the constant domains. Antibodies fragments also include single chain antibodies such as single chain Fv antibodies (scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker as described in Huston, et al. (1988) Proc. Nat. Acad. Sci. USA, 85: 5879-5883 which is incorporated by reference herein. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743, incorporated by reference herein in its entirety). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778, all of which are incorporated by reference herein in their entirety).

The antibody or antibody fragment may be of animal origin such as, by way of example and not limitation, from mouse or rat or human origin or may be chimeric (see e.g. Morrison et al., 1984, Proc. Nat. Acad. Sci. USA 81, 6851-6855) or humanized (see e.g. Jones et al., 1986, Nature 321, 522-525). Methods of producing antibodies suitable for use in the present invention are well known to those skilled in the art and can be found described in such publications as Harlow & Lane, Antibodies: A Laboratory Manual, Cold Spring Harbor Laboratory, 1988. The genes encoding the antibody chains may be cloned in cDNA or in genomic form by any cloning procedure known to those skilled in the art (see e.g. Maniatis et al., Molecular Cloning: A Laboratory Manual, Cold Spring Harbor laboratory, 1982).

Specific antibodies are produced by injecting an antigen into a mammal, such as a mouse, rat or rabbit for smaller quantities of antibody, or goat, sheep, or horse for larger quantities of antibody. Blood isolated from these animals contains polyclonal antibodies, which are a collection of multiple antibodies that bind to the same antigen, in the serum, which is called antiserum. Antigens are also injected into chickens for generation of polyclonal antibodies in egg yolk (see e.g. Tini M, et al. (2002). “Generation and application of chicken egg-yolk antibodies”. Comp. Biochem. Physiol., Part A Mol. Integr. Physiol. 131 (3): 569-74). To obtain antibody that is specific for a single epitope of an antigen, antibody-secreting lymphocytes are isolated from the animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Procedures for preparing antibodies are given in the “Examples” and in Harlow & Lane (1988), Antibodies: A Laboratory Manual, Cold Spring Harbor Laboratory, Chapters 6-8, pp 139-318 and Golding, Monoclonal Antibodies: Principles and Practice, 2 ed., (1986) Academic Press.

“Organophosphate compound” as used herein is a molecule having an structure with a tetrahedral, pentavalent phosphorous atom to which is attached four substituents of which one is a ═O or ═S, one is a leaving group (Z) and two of which are groups X and Y and is sometimes written in typeset as XYP(O)Z or XYP(S)Z, in which the parentheses denotes a pi bond to oxygen or sulfur. Typically, X and Y are independently alkoxy, alkyl thiolester, amine, and the like Oftentimes X and Y are independently methoxy or ethoxy and Z=phenoxy, substituted phenoxy or another good leaving group. Sometimes one of X and Y are an organic moiety having a carbon atom directly attached to the phosphorous atom and the other X and Y is as previously defines. Upon interaction of a polypeptide with an organophosphate compound or a metabolite thereof, substituent Z is replaced with an oxygen-based or nitrogen-based moiety that is derived form a hydroxyl or an amino group of the protein or polypeptide, respectively, to provide a organophosphate biomarker.

Organophosphate compounds (OP-compounds) include organophosphoryl pesticides (OP-insecticides), reactive organophosphoryl compounds and highly reactive organophosphoryl compounds. Typically an OP insecticides contains a P═S moiety (i.e, has the structure XYP(S)Z) that provides hydrolytic and environmental stability, a higher logP (more lipophilic, to penetrate the exoskeleton of insects), has low reactivity toward serine hydrolases (e.g., non-toxic), and superior storage, dispersive and spraying properties. A reactive organophosphoryl compound has the structure XYP(O)Z has does not require metabolic processing to be effective in producing a biomarker (forms a covalent adduct with a protein or polypeptide without requiring prior metabolic activation). An OP-pesticide that contains a P═S substituent (i.e. a organothiophoshoryl pesticide) more readily reacts with a polypeptide after metabolic activation of the organophosphoryl pesticide to a reactive organophorporyl compound, commonly referred to as the oxon (P═O) form of the pesticide. In insects, as in humans, the P═S form is oxidized to the P═O, but this metabolic transformation is significantly faster in insects. It is the oxon form that is mainly responsible for the activity of a pesticide as a suicide inhibitor of acetylcholinesterase (AChE) and other serine hydrolases. In insecticides represented by the formula XYP(S)Z, oftentimes X and Y independently are MeO or EtO. Thus, when a organothiophoshoryl insecticide is metabolically activated, the oxon form so produced interacts with a serine hydrolase, such as an acetylcholinesterase (AchE), resulting in a covalent adduct or biomarker whereby a (MeO)(MeO)P═O or (EtO)(EtO)P═O moiety is typically deposited onto the catalytic serine in the active site of the hydrolase. Thus, unless otherwise stated explicitly or by context, the term organophosphate compound will include organothiophosphoryl compounds and their metabolites. Other biomarkers may also be produced by interaction of the oxon form of an organothiophosphoryl compound with other proteins or polypeptides.

Organophosphoryl pesticides are categorized in various classes and subclasses including organophosphate pesticides, organothiophosphate pesticides, aliphatic thiophosphate pesticides, aliphatic amine thiophosphate pesticides, oxime thiophosphate pesticides, heterocyclic thiophosphate pesticides, which include benzothiopyran, benzotriazine, isoindole, isoxazole, pyrazolopyrimidine, pyrimidine, quinoxaline, thiadiazole, and triazole thiophosphate pesticides, phenyl thiophosphate pesticides, phosphonate pesticides, phosphorothiolate pesticides, such as phenyl ethyl phosphorothiolate and phenyl phenyl phosphorothiolate pesticides, phosphoramidate pesticides, phosphoroamidothiolate pesticides and phosphordiamide pesticides. Specific examples, by illustration and not limitation, of organophosphoryl pesticides are given in Table 1.

A “reactive organophosphoryl” compound as used herein is of a class of organophosphate compounds that has the structure XYP(O)Z with X, Y and Z as previously defined for organophosphate and which do not require metabolic activation or additional metabolic processing to be effective in producing a biomarker (i.e, to form a covalent adduct with a protein or polypeptide). Thus, a reactive organophosphoryl compound encompasses the oxon form or a metabolite of an organothiophosphoryl compound or pesticide. Example reactive organophosphoryl compounds, by way of illustration and not limitation, have the structure represented by the named organophosphoryl compounds in Table 1 except ═O replaces ═S as appropriate.

TABLE 1

| | | Structural |
|---|---|---|
| Common Name | IUPAC or CAS Name | Class/Subclass |
| bromfenvinfos | (EZ)-2-bromo-1-(2,4-dichlorophenyl)vinyl | organophosphate |
| | diethyl phosphate | |
| chlorfenvinphos | (EZ)-2-chloro-1-(2,4-dichlorophenyl)vinyl | organophosphate |
| | diethyl phosphate | |
| crotoxyphos | (RS)-1-phenylethyl 3- | organophosphate |
| | (dimethoxyphosphinoyloxy)isocrotonate | |
| dichlorvos | 2,2-dichlorovinyl dimethyl phosphate | organophosphate |
| dicrotophos | (E)-2-dimethylcarbamoyl-1-methylvinyl | organophosphate |
| | dimethyl phosphate | |
| dimethylvinphos | (Z)-2-chloro-1-(2,4-dichlorophenyl)vinyl | organophosphate |
| | dimethyl phosphate | |
| fospirate | dimethyl 3,5,6-trichloro-2-pyridyl phosphate | organophosphate |
| heptenophos | 7-chlorobicyclo[3.2.0]hepta-2,6-dien-6-yl | organophosphate |
| | dimethyl phosphate | |
| methocrotophos | (E)-2-(N-methoxy-N-methylcarbamoyl)-1- | organophosphate |
| | methylvinyl dimethyl phosphate | |
| mevinphos | (EZ)-2-methoxycarbonyl-1-methylvinyl | organophosphate |
| | dimethyl phosphate | |
| monocrotophos | dimethyl (E)-1-methyl-2- | organophosphate |
| | (methylcarbamoyl)vinyl phosphate | |
| naled | (RS)-1,2-dibromo-2,2-dichloroethyl dimethyl | organophosphate |
| | phosphate | |
| naftalofos | diethyl naphthalimidooxyphosphonate | organophosphate |
| Phosphamidon | (EZ)-2-chloro-2-diethylcarbamoyl-1- | organophosphate |
| | methylvinyl dimethyl phosphate | |
| propaphos | 4-(methylthio)phenyl dipropyl phosphate | organophosphate |
| TEPP | tetraethyl pyrophosphate | organophosphate |
| tetrachlorvinphos | (Z)-2-chloro-1-(2,4,5-trichlorophenyl)vinyl | organophosphate |
| | dimethyl phosphate | |
| dioxabenzofos | (RS)-2-methoxy-4H-1,3,2λ⁵- | Organothio- |
| | benzodioxaphosphinine 2-sulfide | phosphate |
| fosmethilan | S-[N-(2-chlorophenyl)butyramidomethyl] O,O- | Organothio- |
| | dimethyl phosphorodithioate | phosphate |
| phenthoate | S-α-ethoxycarbonylbenzyl O,O-dimethyl | Organothio- |
| | phosphorodithioate | phosphate |
| acethion | S-(ethoxycarbonylmethyl) O,O-diethyl | aliphatic |
| | phosphorodithioate | organothio- |
| | | phosphate |
| amiton | S-2-diethylaminoethyl O,O-diethyl | aliphatic |
| | phosphorothioate | organothio- |
| | | phosphate |
| cadusafos | S,S-di-sec-butyl O-ethyl phosphorodithioate | aliphatic |
| | | organothio- |
| | | phosphate |
| chlorethoxyfos | O,O-diethyl (RS)-O-(1,2,2,2-tetrachloroethyl) | aliphatic |
| | phosphorothioate | organothio- |
| | | phosphate |
| chlormephos | S-chloromethyl O,O-diethyl | aliphatic |
| | phosphorodithioate | organothio- |
| | | phosphate |
| demephion | O,O-dimethyl O-[2-(methylthio)ethyl]phosphorothioate | aliphatic |
| | mixture with O,O-dimethyl | organothio- |
| | S-[2-(methylthio)ethyl] phosphorothioate | phosphate |
| dioxabenzofos | (RS)-2-methoxy-4H-1,3,2λ⁵- | Organothio- |
| | benzodioxaphosphinine 2-sulfide | phosphate |
| demeton | O,O-diethyl O-[2-(ethylthio)ethyl]phosphorothioate | aliphatic |
| | mixture with O,O-diethyl S- | organothio- |
| | [2-(ethylthio)ethyl] phosphorothioate | phosphate |
| demeton-methyl | O-[2-(ethylthio)ethyl] O,O-dimethyl | aliphatic |
| | phosphorothioate mixture with S-[2- | organothio |
| | (ethylthio)ethyl] O,O-dimethyl | phosphate |
| | phosphorothioate | |
| demeton-S- | S-2-ethylsulfonylethyl O,O-dimethyl | aliphatic |
| methylsulphon | phosphorothioate | organothio- |
| | | phosphate |
| disulfoton | O,O-diethyl S-2-ethylthioethyl | aliphatic |
| | phosphorodithioate | organothio- |
| | | phosphate |
| ethion | O,O,O′,O′-tetraethyl S,S′-methylene | aliphatic |
| | bis(phosphorodithioate) | organothio- |
| | | phosphate |
| ethoprophos | O-ethyl S,S-dipropyl phosphorodithioate | aliphatic |
| | | organothio- |
| | | phosphate |
| IPSP | S-ethylsulfinylmethyl O,O-diisopropyl | aliphatic |
| | phosphorodithioate | organothio- |
| | | phosphate |
| Isothioate | S-2-isopropylthioethyl O,O-dimethyl | aliphatic |
| | phosphorodithioate | organothio- |
| | | phosphate |
| malathion | diethyl | aliphatic |
| | (dimethoxyphosphinothioylthio)succinate | organothio- |
| | | phosphate |
| methacrifos | methyl (E)-3-(dimethoxyphosphinothioyloxy)- | aliphatic |
| | 2-methylacrylate | organothio- |
| | | phosphate |
| oxydemeton- | S-2-ethylsulfinylethyl O,O-dimethyl | aliphatic |
| methyl | phosphorothioate | organothio- |
| | | phosphate |
| oxydeprofos | (RS)-{S-[(1RS)-2-(ethylsulfinyl)-1-methylethyl]O, | aliphatic |
| | O-dimethyl phosphorothioate} | organothio- |
| | | phosphate |
| oxydisulfoton | O,O-diethyl S-2-ethylsulfinylethyl | aliphatic |
| | phosphorodithioate | organothio- |
| | | phosphate |
| phorate | O,O-diethyl S-ethylthiomethyl | aliphatic |
| | phosphorodithioate | organothio- |
| | | phosphate |
| sulfotep | O,O,O′,O′-tetraethyl dithiopyrophosphate | aliphatic |
| | | organothio- |
| | | phosphate |
| terbufos | S-tert-butylthiomethyl O,O-diethyl | aliphatic |
| | phosphorodithioate | organothio- |
| | | phosphate |
| thiometon | S-2-ethylthioethyl O,O-dimethyl | aliphatic |
| | phosphorodithioate | organothio- |
| | | phosphate |
| amidithion | S-2-methoxyethylcarbamoylmethyl O,O- | aliphatic amide |
| | dimethyl phosphorodithioate | organothio- |
| | | phosphate |
| cyanthoate | S-[N-(1-cyano-1- | aliphatic amide |
| | methylethyl)carbamoylmethyl] O,O-diethyl | organothio- |
| | phosphorothioate | phosphate |
| dimethoate | O,O-dimethyl S-methylcarbamoylmethyl | aliphatic amide |
| | phosphorodithioate | organothio- |
| | | phosphate |
| ethoate-methyl | S-ethylcarbamoylmethyl O,O-dimethyl | aliphatic amide |
| | phosphorodithioate | organothio- |
| | | phosphate |
| formothion | S-[formyl(methyl)carbamoylmethyl] O,O- | aliphatic amide |
| | dimethyl phosphorodithioate | organothio- |
| | | phosphate |
| mecarbam | S-(N-ethoxycarbonyl-N- | aliphatic amide |
| | methylcarbamoylmethyl) O,O-diethyl | organothio- |
| | phosphorodithioate | phosphate |
| omethoate | O,O-dimethyl S-methylcarbamoylmethyl | aliphatic amide |
| | phosphorothioate | organothio- |
| | | phosphate |
| prothoate | 2-diethoxyphosphinothioylthio-N- | aliphatic amide |
| | isopropylacetamide | organothio- |
| | | phosphate |
| sophamide | S-methoxymethylcarbamoylmethyl O,O- | aliphatic amide |
| | dimethyl phosphorodithioate | organothio- |
| | | phosphate |
| vamidothion | O,O-dimethyl S-(RS)-2-(1- | aliphatic amide |
| | methylcarbamoylethylthio)ethyl | organothio- |
| | phosphorothioate | phosphate |
| chlorphoxim | O,O-diethyl 2-chloro-α- | oxime organothio- |
| | cyanobenzylideneaminooxyphosphonothioate | phosphate |
| phoxim | O,O-diethyl α- | oxime organothio- |
| | cyanobenzylideneaminooxyphosphonothioate | phosphate |
| phoxim-methyl | O,O-dimethyl α- | oxime organothio- |
| | cyanobenzylideneaminooxyphosphonothioate | phosphate |
| azamethiphos | S-6-chloro-2,3-dihydro-2-oxo-1,3-oxazolo[4,5- | heterocyclic |
| | b]pyridin-3-ylmethyl O,O-dimethyl | organothiophosphate |
| | phosphorothioate | |
| coumaphos | O-3-chloro-4-methyl-2-oxo-2H-chromen-7-yl | heterocyclic |
| | O,O-diethyl phosphorothioate | organothio- |
| | | phosphate |
| coumithoate | O,O-diethyl O-(7,8,9,10-tetrahydro-6-oxo-6H- | heterocyclic |
| | benzo[c]chromen-3-yl) phosphorothioate | organothio- |
| | | phosphate |
| coumithoate | O,O-diethyl O-(7,8,9,10-tetrahydro-6-oxo-6H- | heterocyclic |
| | benzo[c]chromen-3-yl) phosphorothioate | organothio- |
| | | phosphate |
| dioxathion | S,S′-(1,4-dioxane-2,3-diyl) O,O,O′,O′- | heterocyclic |
| | tetraethyl bis(phosphorodithioate) | organo- |
| | | thiophosphate |
| endothion | S-5-methoxy-4-oxo-4H-pyran-2-ylmethyl O,O- | heterocyclic |
| | dimethyl phosphorothioate | organo- |
| | | thiophosphate |
| menazon | S-4,6-diamino-1,3,5-triazin-2-ylmethyl O,O- | heterocyclic |
| | dimethyl phosphorodithioate | organo- |
| | | thiophosphate |
| morphothion | O,O-dimethyl S-morpholinocarbonylmethyl | heterocyclic |
| | phosphorodithioate | organo- |
| | | thiophosphate |
| phosalone | S-6-chloro-2,3-dihydro-2-oxo-1,3-benzoxazol- | heterocyclic |
| | 3-ylmethyl O,O-diethyl phosphorodithioate | organo- |
| | | thiophosphate |
| pyraclofos | (RS)-[O-1-(4-chlorophenyl)pyrazol-4-yl O- | heterocyclic |
| | ethyl S-propyl phosphorothioate] | organo- |
| | | thiophosphate |
| pyridaphenthion | O-(1,6-dihydro-6-oxo-1-phenylpyridazin-3-yl)O, | heterocyclic |
| | O-diethyl phosphorothioate | organo- |
| | | thiophosphate |
| quinothion | O,O-diethyl O-2-methyl-4-quinolyl | heterocyclic |
| | phosphorothioate | organo- |
| | | thiophosphate |
| dithicrofos | S-[(RS)-6-chloro-3,4-dihydro-2H-1-benzothiin- | benzothiopyran |
| | 4-yl] O,O-diethyl phosphorodithioate | organo- |
| | | thiophosphate |
| thicrofos | S-[(RS)-6-chloro-3,4-dihydro-2H-1-benzothiin- | benzothiopyran |
| | 4-yl] O,O-diethyl phosphorothioate | organothiophosphate |
| azinphos-ethyl | S-3,4-dihydro-4-oxo-1,2,3-benzotriazin-3- | benzotriazine |
| | ylmethyl O,O-diethyl phosphorodithioate | organo- |
| | | thiophosphate |
| azinphos-methyl | S-3,4-dihydro-4-oxo-1,2,3-benzotriazin-3- | benzotriazine |
| | ylmethyl O,O-dimethyl phosphorodithioate | organo- |
| | | thiophosphate |
| dialifos | S—(RS)-2-chloro-1-phthalimidoethyl O,O- | isoindole |
| | diethyl phosphorodithioate | organothiophosphate |
| phosmet | O,O-dimethyl S-phthalimidomethyl | isoindole organo- |
| | phosphorodithioate | thiophosphate |
| isoxathion | O,O-diethyl O-5-phenyl-1,2-oxazol-3-yl | isoindole organo- |
| | phosphorothioate | thiophosphate |
| zolaprofos | (RS)-(O-ethyl S-3-methyl-1,2-oxazol-5- | isoxazole organo- |
| | ylmethyl S-propyl phosphorodithioate) | thiophosphate |
| chlorprazophos | O-(3-chloro-7-methylpyrazolo[1,5-a]pyrimidin- | pyrazolopyrimidine |
| | 2-yl) O,O-diethyl phosphorothioate | organothio- |
| | | phosphate |
| pyrazophos | ethyl 2-diethoxyphosphinothioyloxy-5- | pyrazolopyrimidine |
| | methylpyrazolo[1,5-a]pyrimidine-6- | organo- |
| | carboxylate | thiophosphate |
| chlorpyrifos | O,O-diethyl O-3,5,6-trichloro-2-pyridyl | pyridine organo- |
| | phosphorothioate | thiophosphate |
| chlorpyrifos- | O,O-dimethyl O-3,5,6-trichloro-2-pyridyl | pyridine organo- |
| methyl | phosphorothioate | thiophosphate |
| butathiofos | O-2-tert-butylpyrimidin-5-yl O,O-diethyl | pyrimidine organo- |
| | phosphorothioate | thiophosphate |
| diazinon | O,O-diethyl O-2-isopropyl-6-methylpyrimidin- | pyrimidine organo- |
| | 4-yl phosphorothioate | thiophosphate |
| etrimfos | O-6-ethoxy-2-ethylpyrimidin-4-yl O,O- | pyrimidine organo- |
| | dimethyl phosphorothioate | thiophosphate |
| pirimiphos-ethyl | O-2-diethylamino-6-methylpyrimidin-4-yl O,O- | pyrimidine organo- |
| | diethyl phosphorothioate | thiophosphate |
| pirimiphos- | O-2-diethylamino-6-methylpyrimidin-4-yl O,O- | pyrimidine organo- |
| methyl | dimethyl phosphorothioate | thiophosphate |
| primidophos | O,O-diethyl O-2-N-ethylacetamido-6- | pyrimidine organo- |
| | methylpyrimidin-4-yl phosphorothioate | thiophosphate |
| pyrimitate | O-2-dimethylamino-6-methylpyrimidin-4-yl | pyrimidine organo- |
| | O,O-diethyl phosphorothioate | thiophosphate |
| tebupirimfos | (RS)-[O-(2-tert-butylpyrimidin-5-yl) O-ethyl O- | pyrimidine |
| | isopropyl phosphorothioate] | organothiophosphate |
| quinalphos | O,O-diethyl O-quinoxalin-2-yl | quinoxailne |
| | phosphorothioate | organo- |
| | | thiophosphate |
| quinalphos- | O,O-dimethyl O-quinoxalin-2-yl | quinoxaline |
| methyl | phosphorothioate | organo- |
| | | thiophosphate |
| athidathion | O,O-diethyl S-2,3-dihydro-5-methoxy-2-oxo- | thiadiazole |
| | 1,3,4-thiadiazol-3-ylmethyl | organo- |
| | phosphorodithioate | thiophosphate |
| lythidathion | S-5-ethoxy-2,3-dihydro-2-oxo-1,3,4- | thiadiazole |
| | thiadiazol-3-ylmethyl O,O-dimethyl | organo- |
| | phosphorodithioate | thiophosphate |
| methidathion | S-2,3-dihydro-5-methoxy-2-oxo-1,3,4- | thiadiazole |
| | thiadiazol-3-ylmethyl O,O-dimethyl | organo- |
| | phosphorodithioate | thiophosphate |
| prothidathion | O,O-diethyl S-2,3-dihydro-5-isopropoxy-2- | thiadiazole |
| | oxo-1,3,4-thiadiazol-3-ylmethyl | organo- |
| | phosphorodithioate | thiophosphate |
| sazofos | O-5-chloro-1-isopropyl-1H-1,2,4-triazol-3-yl | triazole organo- |
| | O,O-diethyl phosphorothioate | thiophosphat |
| triazophos | O,O-diethyl O-1-phenyl-1H-1,2,4-triazol-3-yl | triazole organo- |
| | phosphorothioate | thiophosphat |
| azothoate | O-4-[(EZ)-(4-chlorophenyl)azo]phenyl O,O- | phenyl organothio- |
| | dimethyl phosphorothioate | phosphate |
| bromophos | O-4-bromo-2,5-dichlorophenyl O,O-dimethyl | phenyl organo- |
| | phosphorothioate | thiophosphate |
| bromophos-ethyl | O-4-bromo-2,5-dichlorophenyl O,O-diethyl | phenyl organo- |
| | phosphorothioate | Thiophosphate |
| chlorthiophos | O-[dichloro(methylthio)phenyl] O,O-diethyl | phenyl organo- |
| | phosphorothioate (major component) | thiophosphate |
| cyanophos | O-4-cyanophenyl O,O-dimethyl | phenyl organo- |
| | phosphorothioate | thiophosphate |
| cythioate | O,O-dimethyl O-4-sulfamoylphenyl | phenyl organo- |
| | phosphorothioate | thiophosphate |
| dichlofenthion | O-2,4-dichlorophenyl O,O-diethyl | phenyl organo- |
| | phosphorothioate | thiophosphate |
| etaphos | (RS)-[O-2,4-dichlorophenyl O-ethyl S-propyl | phenyl organo- |
| | phosphorothioate] | thiophosphate |
| famphur | O-4-dimethylsulfamoylphenyl O,O-dimethyl | phenyl organo- |
| | phosphorothioate | thiophosphate |
| fenchlorphos | O,O-dimethyl O-2,4,5-trichlorophenyl | phenyl organo- |
| | phosphorothioate | thiophosphate |
| fenitrothion | O,O-dimethyl O-4-nitro-m-tolyl | phenyl organo- |
| | phosphorothioate | thiophosphate |
| fensulfothion | O,O-diethyl O-4-methylsulfinylphenyl | phenyl organo- |
| | phosphorothioate | thiophosphate |
| fenthion | O,O-dimethyl O-4-methylthio-m-tolyl | phenyl organo- |
| | phosphorothioate | thiophosphate |
| fenthion-ethyl | O,O-diethyl O-4-methylthio-m-tolyl | phenyl organo- |
| | phosphorothioate | thiophosphate |
| heterophos | RS)-(O-ethyl O-phenyl S-propyl | phenyl organo- |
| | phosphorothioate) | thiophosphate |
| jodfenphos | O-2,5-dichloro-4-iodophenyl O,O-dimethyl | phenyl organo- |
| | phosphorothioate | thiophosphate |
| mesulfenfos | O,O-dimethyl O-4-methylsulfinyl-m-tolyl | phenyl organo- |
| | phosphorothioate | thiophosphate |
| parathion | O,O-diethyl O-4-nitrophenyl phosphorothioate | phenyl organo- |
| | | thiophosphate |
| parathion-methyl | O,O-dimethyl O-4-nitrophenyl | phenyl organo- |
| | phosphorothioate | thiophosphate |
| phenkapton | S-2,5-dichlorophenylthiomethyl O,O-diethyl | phenyl organo- |
| | phosphorodithioate | thiophosphate |
| phosnichlor | O-4-chloro-3-nitrophenyl O,O-dimethyl | phenyl organo- |
| | phosphorothioate | thiophosphate |
| profenofos | (RS)-(O-4-bromo-2-chlorophenyl O-ethyl S- | phenyl organo- |
| | propyl phosphorothioate) | thiophosphate |
| prothiofos | RS)-(O-2,4-dichlorophenyl O-ethyl S-propyl | phenyl organo- |
| | phosphorodithioate | thiophosphate |
| sulprofos | (RS)-[O-ethyl O-4-(methylthio)phenyl S-propyl | phenyl organo- |
| | phosphorodithioate] | thiophosphate |
| trichlorfon | dimethyl (RS)-2,2,2-trichloro-1- | phosphonate |
| | hydroxyethylphosphonate | |
| mecarphon | methyl (RS)- | phosphonothioate |
| | {[methoxy(methyl)phosphinothioylthio]acetyl}(methyl)carbamate | |
| fonofos | O-ethyl S-phenyl ethylphosphonodithioate | phenyl ethyl- |
| | | phosphonothioate |
| Trichloronat | O-ethyl O-(2,4,5-trichlorophenyl) | phenyl ethyl- |
| | ethylphosphonothioat | phosphonothioat |
| cyanofenphos | (RS)-(O-4-cyanophenyl O-ethyl | phenyl phenyl- |
| | phenylphosphonothioate) | phosphonothioate |
| fenamiphos | (RS)-(ethyl 4-methylthio-m-tolyl | phosphoramidate |
| | isopropylphosphoramidate) | |
| fosthietan | diethyl 1,3-dithietan-2- | phosphoramidate |
| | ylidenephosphoramidate | |
| isocarbophos | (RS)-(O-2-isopropoxycarbonylphenyl O- | phosphoramidothioate |
| | methyl phosphoramidothioate) | |
| mephosfolan | diethyl [(EZ)-4-methyl-1,3-dithiolan-2- | phosphoramidate |
| | ylidene]phosphoramidate | |
| phosfolan | diethyl 1,3-dithiolan-2- | phosphoramidate |
| | ylidenephosphoramidate | |
| isofenphos | (RS)-(O-ethyl O-2-isopropoxycarbonylphenyl | Phosphoramido- |
| | isopropylphosphoramidothioate) | thioate |
| pirimetaphos | (RS)-(2-diethylamino-6-methylpyrimidin-4-yl | phosphoramidate |
| | methyl methylphosphoramidate) | |
| acephate | (RS)-(O,S-dimethyl | Phosphoramido- |
| | acetylphosphoramidothioate) | thioate |
| isocarbophos | (RS)-(O-2-isopropoxycarbonylphenyl O- | Phosphoramido- |
| | methyl phosphoramidothioate) | thioate |
| isofenphos | (RS)-(O-ethyl O-2-isopropoxycarbonylphenyl | Phosphoramid- |
| | isopropylphosphoramidothioate) | othioate |
| methamidophos | RS)-(O,S-dimethyl phosphoramidothioate) | phosphoramidothioate |
| propetamphos | (RS)-[(E)-O-2-isopropoxycarbonyl-1- | Phosphoramido- |
| | methylvinyl O-methyl | thioate |
| | ethylphosphoramidothioate] | |
| dimefox | tetramethylphosphorodiamidic fluoride | phosphorodiamide |
| mazidox | tetramethylphosphorodiamidic azide | phosphorodiamide |
| mipafox | N,N′-diisopropylphosphorodiamidic fluoride | phosphorodiamide |
| schradan | octamethylpyrophosphoric tetraamide | phosphorodiamide |

“Reactive carbamate compound” as used here is a compound containing a carbamate moiety that is capable of reacting with a polypeptide-based nucleophile such as an amino group and includes the ε-amino group of a lysine residue, the guanidine group of an arginine reside or the amino group of the N-terminal amino acid residue of a polypeptide without requiring prior metabolic activation. Non-limiting examples of reactive carbamate compounds which are used as pesticides are given in Table 3. A reactive carbamate compound or pesticide will provide a biomarker (i.e. a covalent adduct) whose structure will be dependent on the polypeptide nucleophile (NH₂ or OH) that reacts to provide the covalent adduct and the leaving group on the reactive carbamate compound that participates (i.e. is lost) in the formation of the covalent adduct. Typically the covalent adduct from interaction of a reactive carbamate compound with a polypeptide-based nucleophile will contain a urethane or urea moiety as further described herein for carbamate biomarkers. It is within the ability of the skilled artisan to predict with reasonable assurance the type of covalent adduct (i.e., urethane or urea) to be formed based upon the structure of the reactive carbamate compound and the polypeptide-based nucleophile involved.

TABLE 3

| | | Structural |
|---|---|---|
| Common Name | IUPAC Name | Class/subclass |
| carbaryl | 1-naphthyl methylcarbamate | carbamate |
| bendiocarb | 2,2-dimethyl-1,3-benzodioxol-4-yl | carbamate |
| | methylcarbamate | |
| benfuracarb | ethyl N-[2,3-dihydro-2,2-dimethylbenzofuran- | benzofuranyl |
| | 7-yloxycarbonyl(methyl)aminothio]-N- | methylcarbamate |
| | isopropyl-β-alaninate | |
| carbofuran | 2,3-dihydro-2,2-dimethylbenzofuran-7-yl | benzofuranyl |
| | methylcarbamate | methylcarbamate |
| carbosulfan | 2,3-dihydro-2,2-dimethylbenzofuran-7-yl | benzofuranyl |
| | (dibutylaminothio)methylcarbamate | methyllcarbamate |
| decarbofuran | (RS)-2,3-dihydro-2-methylbenzofuran-7-yl | benzofuranyl |
| | methylcarbamate | methylcarbamate |
| furathiocarb | butyl 2,3-dihydro-2,2-dimethylbenzofuran-7-yl | benzofuranyl |
| | N,N′-dimethyl-N,N′-thiodicarbamate | methylcarbamate |
| dimetan | 5,5-dimethyl-3-oxocyclohex-1-enyl | dimethylcarbamate |
| | dimethylcarbamate | |
| dimetilan | 1-dimethylcarbamoyl-5-methylpyrazol-3-yl | pyrazole |
| | dimethylcarbamate | |
| hyquincarb | 5,6,7,8-tetrahydro-2-methyl-4-quinolyl | dimethylcarbamate |
| | dimethylcarbamate | |
| pirimicarb | 2-dimethylamino-5,6-dimethylpyrimidin-4-yl | dimethylcarbamate |
| | dimethylcarbamate | |
| alanycarb | ethyl (Z)-N-benzyl-N-[[methyl(1- | oxime carbamate |
| | methylthioethylideneaminooxycarbonyl)amino]thio]- | |
| | β-alaninate | |
| aldicarb | (EZ)-2-methyl-2-(methylthio)propionaldehyde | oxime carbamate |
| | O-methylcarbamoyloxime | |
| aldoxycarb | (EZ)-2-mesyl-2-methylpropionaldehyde O- | oxime carbamate |
| | methylcarbamoyloxime | |
| butocarboxim | (EZ)-3-(methylthio)butanone O- | oxime carbamate |
| | methylcarbamoyloxime | |
| butoxycarboxim | (EZ)-3-mesylbutanone O- | oxime carbamate |
| | methylcarbamoyloxime | |
| methomyl | S-methyl (EZ)-N- | oxime carbamate |
| | (methylcarbamoyloxy)thioacetimidate | |
| nitrilacarb | (EZ)-4,4-dimethyl-5- | oxime carbamate |
| | (methylcarbamoyloxyimino)valeronitrile | |
| oxamyl | EZ)-N,N-dimethyl-2- | oxime carbamate |
| | methylcarbamoyloxyimino-2- | |
| | (methylthio)acetamide | |
| tazimcarb | (EZ)-N-methyl-1-(3,5,5-trimethyl-4-oxo-1,3- | oxime carbamate |
| | thiazolidin-2-ylideneaminooxy)formamide | |
| thiocarboxime | (EZ)-3-[1- | oxime carbamate |
| | (methylcarbamoyloxyimino)ethylthio]propiono | |
| | nitrile | |
| thiodicarb | (3EZ,12EZ)-3,7,9,13-tetramethyl-5,11-dioxa- | oxime carbamate |
| | 2,8,14-trithia-4,7,9,12-tetraazapentadeca- | |
| | 3,12-diene-6,10-dione | |
| aminocarb | 4-dimethylamino-m-tolyl methylcarbamate | phenyl methyl- |
| | | carbamate |
| bufencarb | (RS)-3-(1-methylbutyl)phenyl | phenyl methyl- |
| | methylcarbamate and 3-(1-ethylpropyl)phenyl | carbamate |
| | methylcarbamate | |
| butacarb | 3,5-di-tert-butylphenyl methylcarbamate | phenyl methyl- |
| | | carbamate |
| carbanolate | 6-chloro-3,4-xylyl methylcarbamate | phenyl methyl- |
| | | carbamate |
| cloethocarb | (RS)-2-(2-chloro-1-methoxyethoxy)phenyl | phenyl methyl- |
| | methylcarbamate | carbamate |
| dicresyl | cresyl methylcarbamate | phenyl methyl- |
| | | carbamate |
| dioxacarb | 2-(1,3-dioxolan-2-yl)phenyl methylcarbamate | phenyl methyl- |
| | | carbamate |
| EMPC | 4-ethylthiophenyl methylcarbamate | phenyl methyl- |
| | | carbamate |
| ethiofencarb | α-ethylthio-o-tolyl methylcarbamate | phenyl methyl- |
| | | carbamate |
| fenethacarb | 3,5-diethylphenyl methylcarbamate | phenyl methyl- |
| | | carbamate |
| fenobucarb | (RS)-2-sec-butylphenyl methylcarbamate | phenyl |
| | | methylcarbamate |
| soprocarb | o-cumenyl methylcarbamate | phenyl methyl- |
| | | carbamate |
| methiocarb | 4-methylthio-3,5-xylyl methylcarbamate | phenyl methyl- |
| | | carbamate |
| metolcarb | m-tolyl methylcarbamate | phenyl methyl- |
| | | carbamate |
| mexacarbate | 4-dimethylamino-3,5-xylyl methylcarbamate | phenyl methyl- |
| | | carbamate |
| promacyl | 5-methyl-m-cumenyl | phenyl methyl- |
| | butyryl(methyl)carbamate | carbamate |
| promecarb | 5-methyl-m-cumenyl methylcarbamate | phenyl methyl- |
| | | carbamate |
| Propoxur | 2-isopropoxyphenyl methylcarbamate | phenyl methyl- |
| | | carbamate |
| thiofanox | (EZ)-3,3-dimethyl-1-methylthiobutanone O- | phenyl methyl- |
| | methylcarbamoyloxime | carbamate |
| trimethacarb | 2,3,5(or 3,4,5)-trimethylphenyl | phenyl methyl- |
| | methylcarbamate | carbamate |
| XMC | 3,5-xylyl methylcarbamate | phenyl |
| | | methylcarbamate |
| Xylylcarb | 3,4-xylyl methylcarbamate | phenyl methyl- |
| | | carbamate |

“Biomarker” as used herein means a molecule that is predictive or diagnostic for a disease state in a mammal or is required for persistence or progression of the disease state or results from a chemical or biological insult or exposure to the mammal. A biomarker may be a naturally occurring molecule in the mammal but is present in a biological compartment in which the biomarker is not normally present or is present in a concentration for a period of time not associated with the maintenance of well-being or is associated with or predictive of a disease state. A biomarker may come from or be produced by a foreign object such as a bacteria, fungus, virus or prion. A biomarker may result from a chemical insult or an environmental toxin and may come from, by way of example and not limitation, an exposure of a mammal to a synthetic chemical, environmental toxin or to a compound in nature in an amount and within a period of time predictive to be harmful to the health of a mammal. The exposure may be acute with immediate or rapid onset of a disease state or a symptom thereof or may be chronic and is associated with a slower progression of or to a disease state or a slower manifestation of a symptom related to the disease state.

In one embodiment biomarkers are derived from suicide inhibition of a serine hydrolase, defined elsewhere in the specification, and are named analogously and for example include butyryl cholinesterase biomarkers, acetylcholinesterase biomarkers and choline esterase biomarkers. Organophosphate biomarkers from suicide inhibition of an enzyme are alternatively called an OP-conjugate of the specific enzyme inhibited such as an acetylcholinesterase-OP conjugate (OP-AChE conjugate). Also contemplated are biomarker which are derived from suicide inhibition of a hydrolase enzyme or an enzyme within a enzyme class define by EC 3.1.1 (carboxylic ester hydrolases), EC 3.1.2 (thioester hydrolyases), EC 3.1.3 (phosphoric monoester hydrolases), EC 3.1.4 (phosphoric diester hydrolases), EC 3.1.5 (triphosphoric monoester hydrolases), EC 3.1.6 (sulfuric ester hydrolases), EC 3.1.7 (diphosphoric monoester hydrolases) EC 3.1.8 (phosphoric triester hydrolases) and exonucleases under EC 3.1.11, EC 3.1.13, EC 3.1.14 and EC 3.1.15.

A biomarker may be a polypeptide, including but not limited to a polypeptide comprising or consisting of a cellular receptor or an enzyme, modified by an exposure to a synthetic chemical or an environmental toxin, wherein the synthetic chemical is an organophosphate compound or a reactive carbamate compound. In another embodiment a biomarker is an enzyme or a fragment thereof modified by an exposure to a synthetic chemical, wherein the synthetic chemical is a suicide inhibitor of the enzyme. In one embodiment the biomarker results from exposure of a polypeptide to an organophosphate compound wherein the organophosphate compound is a reactive organophosphoryl compound, an organophosphoryl insecticide, pesticide or a metabolite thereof. In another embodiment a biomarker results from exposure of a polypeptide to a reactive carbamate compound or a metabolite thereof. In one embodiment a biomarker results from exposure of a polypeptide or a protein in vitro or in vivo (i.e, a polypeptide or protein in a mammal) to an organophosphate compound or a metabolite thereof. A biomarker so produced will be comprised a polypeptide, wherein the polypeptide is comprised or consists of a protein, and a phosphorous containing moiety derived from the organophosphate compound wherein the phosphorous containing residue is covalently attached to the polypeptide.

An organophosphate biomarker comprises a polypeptide and a phosphorous containing moiety wherein the phosphorous containing moiety is derived from an organophosphate compound and is covalently attached to the polypeptide. The definition of organophosphate biomarker is independent of the manner in which it is produced. Sometimes an organophosphate compound interacts with a polypeptide or a protein in a mammal to produce a biomarker. Sometimes a biomarker is produced from an organophosphate compound that has interacted with a polypeptide which comprises or consists of a protein found in nature or with a polypeptide that is isolated or derived from nature. Sometimes a biomarker is produced from an organophosphate compound that has interacted with a polypeptide wherein the polypeptide is a fragment, or a synthetic analog thereof, of a polypeptide found in nature.

Typically, a biomarker from interaction of an organophosphate compound with a polypeptide has a structure of XYP(W)—O— wherein W is ═O or ═S and X, Y, which are substituents bonded to a phosphorous atom, are as defined elsewhere in the specification for organophosphate compounds. Sometimes, an organophosphate biomarker has a structure of (RO)(RO)P(O)—O-polypeptide or (⁻O)(OR)P(O)—O-polypeptide (i.e. a phosphate ester), wherein R are independently selected organic moiety and —O-polypeptide represents a polypeptide that has been modified by the organophosphate compound or a metabolite thereof wherein —O— is derived from a hydroxyl group of an amino acid residue of the polypeptide. Sometimes a biomarker having the structure of (RO)(OR)P(O)—O-polypeptide is initially formed to provide a primary organophosphate biomarker which then undergoes transformation to another covalent adduct now having the structure (O)(R)P(O)—O-polypeptide to provide a secondary organophosphate biomarker.

Sometimes, an organophosphate biomarker has a structure of (RO)(R)P(O)—O-polypeptide or (⁻O)(OR¹)P(O)—O-polypeptide wherein an organic moiety (R) is bonded through carbon to the phosphorous atom (i.e, a phosphonate). Oftentimes the hydroxyl group modified by an organophosphate compound or metabolite thereof belongs to a serine residue; however, modification of the hydroxyl group of other hydroxyl-bearing amino acid residue such as a threonine or tyrosine residue may also provide a polypeptide-based biomarker. In one embodiment each R in the aforementioned structures is independently selected C1-6 alkyl. Sometimes a biomarker having the structure of (RO)(R)P(O)—O-polypeptide is initially formed (i.e., a primary biomarker) which then undergoes transformation to a biomarker having the structure (⁻O)(R)P(O)—O-polypeptide (i.e., a secondary biomarker).

Sometimes a biomarker from exposure to a organophosphate compound has a structure of formula (RO)(RO)P(O)—NH-polypeptide or (⁻O)(RO)P(O)—NH-polypeptide (i.e. a phosphoramidate) wherein R are independently selected organic moiety and —NH-polypeptide represents a polypeptide that has been modified by the organophosphate compound or a metabolite thereof wherein —NH— is derived an amino group of a side chain of an amino acid residue of the polypeptide or the N-terminal amino acid residue of the polypeptide. Sometimes the amino group of an amino acid side chain modified by an organophosphate compound or a metabolite thereof belongs to a lysine or an arginine residue. Sometimes a biomarker having the structure of (RO)(RO)P(O)—NH-polypeptide is initially formed to provide a primary biomarker which then undergoes transformation to another covalent adduct having the structure (⁻O)(R)P(O)—O-polypeptide, which provides a secondary biomarker

In one embodiment a protein modified by an organophosphate compound is a serine hydrolase or a choline esterase including, but not limited to carboxylesterase, butyrylesterase or acetylcholinesterase. In one embodiment a biomarker from an exposure of a choline sterase to an organophosphate or a metabolite thereof has the formula of (RO)(RO)P(O)—O-polypeptide, (⁻O)(RO)P(O)—O-polypeptide, (RO)(R)P(O)—O-polypeptide or (⁻O)(R)P(O)—O-polypeptide wherein R is independently selected organic moiety and —O-polypeptide represents a polypeptide comprising or consisting of the cholinesterase wherein the phosphorus-oxygen bond is between a phosphorous containing residue derived from the organophosphate compound or a metabolite thereof and a hydroxyl group of an amino acid residue of the polypeptide wherein the amino acid residue corresponds to the active site serine of the cholinesterase. A polypeptide modified by an organophosphate compound wherein the polypeptide is derived from or is analogous to an amino acid sequence of a polypeptide found within acetylcholinesterase that contains the active site serine residue is referred to as an OP-AChE conjugate.

In another embodiment a biomarker results from modification of a reactive carbamate compound with a hydroxyl or amino group of a polypeptide. When a reactive carbamate reacts with a polypeptide hydroxyl group, a biomarker with a urethane structure is formed whereas modification of a polypeptide amino group provides a biomarker with a urea structure. A biomarker obtained from reaction of a carbamate with a polypeptide is referred to as a carbamate biomarker. Structures of carbamate biomarkers are represented by formula RN(R)—C(O)—O-polypeptide or RN(R)—C(O)—NH-polypeptide wherein R are independently selected organic moiety and —O-polypeptide and —NH-polypeptide are as described for polypeptides modified by organophosphate compounds.

An organophosphate biomarker or carbamate biomarker may be the covalent adduct initially formed by reaction of an organophosphate compound or a metabolite thereof or a reactive carbamate with a polypeptide or may result from further reaction(s) or processing of the covalent adduct. For example, the initially formed covalent adduct from an organophosphate compound may undergo spontaneous hydrolysis which cleaves a P—O bond substituent (other than the P—O-polypeptide bond) to give a biomarker that is detected by an optical sensor. In some embodiments the initially formed adduct is processed by proteolysis to provide a fragment of the polypeptide that retains the organophosphate or reactive carbamate derived moiety and is the biomarker that is detected by an optical sensor.

“Biomarker receptor” as used herein means a receptor as defined elsewhere in the specification that is immobilized to a biopolymer material by means described elsewhere in the specification and is capable, after being so immobilized, of binding a biomarker to provide for an optical sensor of the biomarker. Biomarker receptors include but are not limited to polypeptides comprising or consisting of cellular receptors and antibodies and are further described elsewhere in the specification

“Immobilization” as used here refers to the attachment or entrapment, either by covalent binding or non-covalent binding of a material to another entity (e.g., a biopolymer material to a solid support) in a manner that restricts the movement of the material.

“Hydrophillic” as used herein in describing a molecule, refers to a molecule that is substantially attracted to water by non-covalent interactions including, but not limited to, hydrogen-bonding, van der Waals' forces, ionic interactions.

“Hydrophobic” as used herein in describing a molecule refers a molecule that associate with other hydrophobic molecules or entities, that results in the exclusion of water.

“Non-covalent binding” as used herein refers to attachment of one molecule to another molecule exclusively through-space interactions that include hydrogen bonding, van der Waals interactions, ionic interactions or combinations thereof.

“Covalent binding” as used herein refers to the attachment of two molecules through one or more covalent bonds.

“Non-releasably” or “non-releasable” as used herein describes an indirect covalent or non-covalent binding (i.e attachment of a molecule such as a biomarker receptor with an entity such as a biopolymer material) by a linker that is resistant to proteolytic or hydrolytic degradation. Typically a non-releasing linker will have a functional group that connects for example a biomarker receptor to a linker group defined elsewhere in the specification through amide, carbamate, hindered ester, urea, disulfide, hydrazone or any other hydrolytically resistant functional group.

“Immobilization means” refers to the structure underlying the immobilization of a molecule such as a biomarker receptor to an entity such as a biopolymer material. For attachment (i.e., immobilization) of a biological-based or derived biomarker receptor such as a cellular biomarker receptor or an antibody biomarker receptor, one or more polypeptides comprising a cellular receptor, a cellular receptor fragment, a ligand of a cellular receptor, a ligand fragment, or an antibody or a fragment thereof is attached to a biopolymer material by a direct or indirect covalent binding or by a non-covalent binding. “Linker” in the context of a direct or an indirect covalent binding of a molecule such as a biomarker to an entity such as a biopolymer material is described in the definition of Linker

“Immobilization means by direct covalent binding” refers to the structure underlying the attachment (i.e. immobilization) of a molecule such as a biomarker receptor by direct covalent binding to another entity such as a biopolymer material without the use of a linker and uses for example a first functional group present or incorporated into a biomarker receptor and second functional group present or incorporated into a biopolymer material that are combined in a manner that results in a functional group, which may be the same type as previously present on the biomarker receptor or biopolymer material or a different type of functional group, that covalently binds the biomarker receptor to the biopolymer material and does not use a linker precursor. For example, a biopolymer material may have a hydrazone group which combined with a biomarker receptor having an aldehyde group which, by an exchange process, forms a new hydrazone group that immobilizes the biomarker receptor to the biopolymer material by direct covalent binding.

“A biomarker receptor immobilization means by direct covalent binding” as used here refers to the underlying structure for attaching a biomarker receptor to a biopolymer material by covalent binding without the use of an intervening linker. In some embodiments, the biomarker receptor is comprised or consists of a polypeptide and the polypeptide is immobilized through one or more sulfhydryl groups in the polypeptide. One or more sulfhydryl groups are introduced into the polypeptide by reducing a native cystine residue (i.e a —S—S— bond) in the polypeptide or by chemically introducing sulfhydryl group(s) by a chemical agent such as 2-iminothiolane. Conjugation (i.e., covalent attachment) of the polypeptide to a biopolymer material is then achieved using a heterobifunctional reagent selective for an amino group, present on the biopolymer material, and a free sulfhydryl group introduced on the polypeptide (see, eg. Aslam, M. and Dent, A. (1998). An example heterobifunctional reagent selective for a sulfhydryl group and an amino group is succinimidyl-4-(N-maleimidomethyl)cyclohexana-1-carboxylate (SMCC) for example. In one embodiment, the reagent N-succinimidyl 3-(2-pyridyldithio)-propionate (SPDP) is used to introduce a thiol group to a polypeptide by cleavage of the thiopyridyl group in a polypeptide-SPDP adduct using dithiothreitol (DTT). SPDP then is used in a second reaction to link the functionalized polypeptide to a thiol functional group of the biopolymer material by forming a mixed disulfide between the thiol group of the biopolymer material and the polypeptide-SPDP adduct with the release of another thiopyridyl group (see, e.g., Wong, Chemistry of protein conjugation and cross-linking, CRC Press (1993) which is incorporated by reference herein). In another embodiment, an activated ester is formed with N-hydroxysuccinimide (NHS) using a carboxylic acid group on the biopolymer material to conjugate the polypeptide thorough one of its amino groups (e.g., epsilon amino group in a lysine or the terminal amino group) by treating the amino group with the activated ester introduced into the biopolymer material. Alternatively an amino moiety of the biopolymer material reacts with an activated ester in the polypeptide (e.g., the ester is formed from the C-terminal carboxyl group and/or a carboxyl group in aspartate or glutamate amino acid side chains). In certain embodiments the hydroxyl group of a hydroxylysine or the hydroxyl group of an N-terminal serine or threonine is converted to an aldehyde using an oxidizing agent such as NaIO₄ and reacted with a hydrazide functionality introduced into the biopolymer material such that a hydrazone linkage is formed (see, e.g. Geoghean, K. F.; Stroh, J. G. Bioconjugate Chem., 3: 138 (1992)). Alternatively, an aldehyde group on a biopolymer material may be condensed with an amino group of a polypeptide to form an imino group which is then reduced by a hydride reducing agent to provide a more hydrolytically stable C—N bond through a process known as reductive amination.

“An indirect immobilization means by covalent binding” as used herein refers to the underlying structure that includes a linker which attaches (i.e., immobilizes) a molecule such as a biomarker receptor to a biopolymer material, wherein the linker covalently binds the biomarker receptor through a first functional group, referred to as a first linker functional group, and covalently binds the biopolymer material through a second functional group, referred to as a second linker functional group, wherein there is a linking group between said first and second functional groups. The intervening linker is incorporated into the underlying structure through use of a linker precursor wherein the linker precursor has appropriate functional groups, referred to as linker precursor functional groups, for forming the first and second functional groups within the linker. The definition of “linker” and linker “precursor” is further described in the definition of Linker

“Immobilization means by non-covalent binding” refers to the underlying structure for attaching (i.e., immobilizing) a molecule such as a biomarker receptor to an entity such as a biopolymer material through non-covalent interactions (i.e., no covalent binding in the underlying structure is responsible for the immobilization). For example, a biotinylated polypeptide can be prepared by chemical modification (e.g., using biotin-NHS(N-hydroxy-succinimide) and/or a biotinylation kit (Pierce Chemicals, Rockford, Ill.)) or by recombinant procedures (e.g., pcDNA™6 BioEase™ Gateway® Biotinylation System, Invitrogen, Inc.) and linked to a streptavidin-derivitized biopolymer material.

“Ligand” as used herein is a molecule that interacts with a cellular receptor by binding to the receptor with a K_{d} in a K_{d} range 20×E-06 M to 1×E-15 M, 10×10E-06 M to 1×10E-12 M, 1×10E-06 to 1×10E-10 or 0.1×10E-06 to 1×10E-9 and wherein the ligand induces the cellular receptor to transmit a biochemical or physiochemical signal upon binding of the ligand that is indicative of the cellular receptor-ligand interaction.

A ligand sometimes comprises or consists of an amino acid sequence that is substantially identical to the sequence of a native ligand (cellular ligand) that binds to any of the cellular receptors described herein or is a fragment thereof wherein the ligand fragment comprises the binding epitope found in the intact cellular ligand that is recognized by the cellular receptor. In one embodiment, a cellular ligand is immobilized to a biopolymer material for detection of a biomarker and thus the cellular ligand becomes a biomarker receptor. In another embodiment, the presence of a ligand, or an increased or decreased amount of the ligand from normal physiology, is indicative of a disease state of or a chemical or biological insult to a mammal and thus the ligand becomes a biomarker for a disease state or insult. The definition of linker encompasses linker fragment unless otherwise specified explicitly or by context.

“Linker” as used herein, refers to the intervening atoms, when present, between a biomarker receptor and a biopolymer material in an optical sensor. The term “linker” herein also refers to any moiety (i.e. linker moiety) that non-releasably connects the biomarker receptor to the biopolymer material.

The linking moiety can be a functional group that covalently binds the biomarker receptor directly to a biopolymer material (i.e without the involvement of intervening atoms between the functional group and the biopolymer material) to provide a structure represented by the formula BMR-BIOM wherein BMR is a biomarker receptor and BIOM is a biopolymer material. Thus, BMR-BIOM represents the structure underlying a direct biomarker receptor immobilization attachment means. In one embodiment, direct covalent binding of the biomarker receptor is by a carbamate, amide, urea or disulfide functional group. All or some of the atoms defining the functional group may be contributed by the biomarker receptor or the biopolymer material. Typically, both the biomarker receptor and biopolymer material will contribute atoms defining the functional group (e.g, an amide linking moiety is formed using a carboxylic acid from the biopolymer material and an amino group from the biomarker receptor or a disulfide is formed between sulfhydryl groups on the biomarker receptor and biopolymer material).

A linking moiety may also contain a series of interposed covalently bonded atoms and their substituents (i.e. interposed atoms) between a biomarker receptor and a biopolymer material in an optical sensor, and are collectively referred to as a linking group or a spacer. Such linking moieties are thus characterized by a first covalent bond or a chemical functional group, referred to as a first linker functional group, that connects the biomarker receptor to a first end of the linker group and a second covalent bond or chemical functional group, referred to as a second linker functional group, that connects the second end of the linker group to the biopolymer material and the interposed atoms. The linker moiety therefore is defined by the linking group, the first linker functional group and the second linker functionality group. In some embodiments the first linker functional group that connects the biomarker to the first end of the linker group and the second functional group that connects the biopolymer to the second end of the linker group are independently a carbamate, amide, disulfide or a succinimidyl group (from conjugate addition of a thiol in a biomarker receptor or biopolymer to a maleimido group in a linker precursor). As used herein, the linker moiety in an optical sensor contains interposed atoms between the biomarker receptor and the biopolymer material and the identities of these interposed atoms are independent of their source and the reaction sequence or sequences that connect the biomarker receptor to the biopolymer material. Use of a linker containing a linker group that immobilizes a biomarker receptor to a biopolymer material provides an indirect attachment between the biomarker receptor and the biopolymer material.

“Linker precursor”, used interchangeably with “linker precursor moiety”, is a compound that is used in immobilization of a biomarker receptor to a biopolymer material by an indirect covalent bonding in which a linker becomes combined with the biomarker receptor and the biopolymer material by covalent binding to provide a structure having the formula BMR-L-BIOM wherein BMR is a biomarker receptor, L is a linker and BIOM is a biopolymer material. Thus, BMR-L-BIOM is the structure underlying an indirect receptor immobilization means of attachment.

Sometimes the optical sensor incorporates a linker containing a linker group to improve binding between a biomarker receptor and a biomarker by relieving steric interactions between the biomarker receptor and the biopolymer material that would perturb unfavorably the conformation of the biomarker receptor or which would inhibit approach of the biomarker to the binding pocket of the biomarker receptor.

In one embodiment a structure having the BMR-L-BIOM is formed using an intermediate having the structure FG1-L′-BIOM or an intermediate having the structure BMR-L′-FG2, wherein L′ represents a linker group, FG1 represents a first linker precursor functional group and FG2 represents a second linker precursor functional group In this embodiment the linker precursor is a compound having two functional groups, referred to as linker precursor functional groups (FG1 and FG2), separated by interposed covalently bonded atoms that become the linker group (or spacer) in a linker, wherein at least one of the linker precursor functional groups is capable of reacting with a biomarker receptor functional group present on the biomarker receptor and at least one of the linker precursor functional groups is capable of reacting a biopolymer material functional group. The first linker functional group and the second linker functional group that comprise the linker formed by the combination of linker precursor functional groups with the biomarker receptor and biopolymer material functional groups may be the same or different and may incorporate one or more atoms that were present in the linker precursor functional groups and-or in the biomarker receptor or biopolymer material prior to the combination that formed the intervening linker. In the embodiment described immediately above, a single linker precursor group gives rise to a linker (L) in a structure having the formula BMR-L-BIOM.

Sometimes the linker precursor resides in two separate molecules that are then brought together to form a structure having the formula BMR-L-BIOM wherein the linker L covalently binds a biomarker receptor to a biopolymer material. Thus, intermediates FG1-L′-BIOM and BMR-L″-FG2 are prepared, wherein L′ and L″ will comprise a linker (L), and are reacted together to provide a structure having the formula BMR-L-BIOM, an example of which is provided by the following scheme.

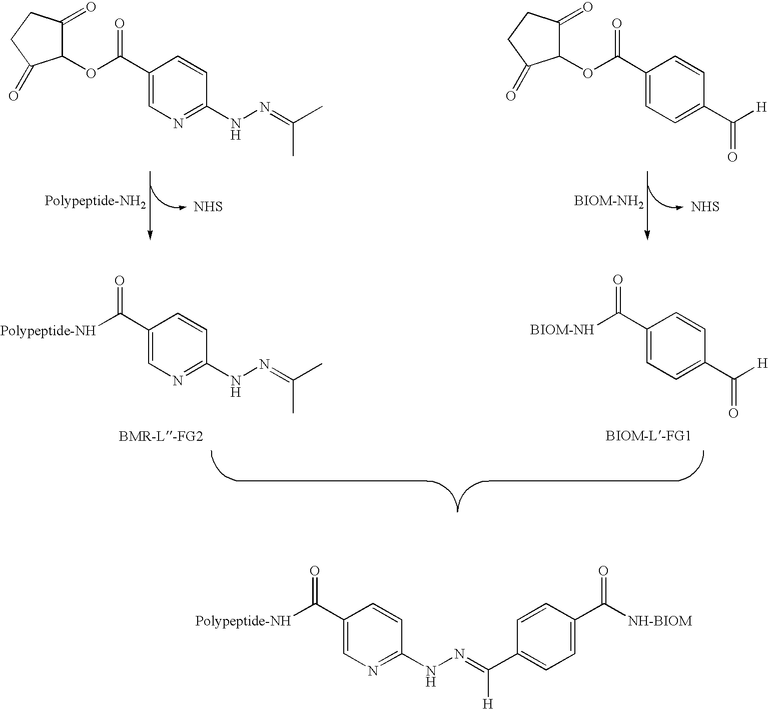

Functional groups that directly connect a biomarker receptor to a biopolymer material or are contained in a linker moiety that indirectly binds a biomarker receptor to a biopolymer material include ester, amide, imine (with subsequent reduction), carbamate, urea, disulfide, succinimidyl, carbonate, sulfonamide, hydrazone, ether, phosphoester, phosphonate, thiophosphonate or phosphoramidate. Choice of appropriate functional groups to be incorporated into binding (either directly or indirectly by an intervening linker) of a biomarker receptor to a biopolymer material will depend on the ease of incorporating the appropriate functional group precursors into the biomarker receptor and biopolymer material and the thermal or hydrolytic stability required for an optical sensor for its intended use. For example, covalent binding of DNA or RNA to a biopolymer material will typically employ phosphoester or phosphoramidate, and for carbonyl-based functional groups where resistance to hydrolysis is required, covalent binding by amide, carbamate or urea would be chosen for example over ester or carbonate.

In one embodiment an optical sensor comprises a biomarker receptor, biopolymer material and a linker wherein the linker non-releasably connects the biomarker receptor to the biopolymer material and is defined by a structure having the formula wherein BMR-L-BIOM wherein BMR is a biomarker receptor, L is a linker and BIOM is a biopolymer material. In one embodiment a BMR-L-BIOM is constructed through preparation of a FG1-L′-BIOM or a BMR-L′-FG2 intermediate, wherein FG1 represents a first linker precursor functional group and FG2 represents a second linker functional group. Reaction between FG1 and FG2 in the above structure creates the linker L which indirectly immobilizes the biomarker receptor to the biopolymer material

Sometimes the linker precursor resides in two separate molecules that are then brought together to form a structure having the formula BMR-L′-L″-BIOM or BRM-L-BIOM wherein the linker wherein L′ and L″ associates noncovalently to form a linker L that noncovalently binds a biomarker receptor (BMR) to a biopolymer material (BIOM). Thus, intermediates L′-BIOM and BMR-L″ are brought together whereupon L′ and L″ contain functional groups capable of interacting non-covalently. Therefore, BMR-L-BIOM in this embodiment represents the underlying structure of an indirect biomarker receptor immobilization attachment means by noncovalent binding.

Functional groups in the various functional group combinations described immediately above for noncovalent attachment of a biomarker receptor to a biopolymer material are chosen so that a linker in BMR-L-BIOM having noncovalent bonding within the linker has a number of noncovalent bonding interactions that afford a nonreleasable linker or a biopolymer material is immobilized to a biopolymer material to provide a structure having the formula BMR-BIOM so that BMR is directly and non-releasably attached (i.e., immobilized) to the biopolymer material. Typically, a functional groups involved in noncovalent binding of a biomarker receptor to a biopolymer material either directly to provide a structure of formula BMR-BIOM or indirectly to provide a structure of formula BMR-L-BIOM will have multiple groups each characterized as a hydrogen bond donor and-or acceptor.

By way of example and not limitation, an indirect noncovalent attachment may involve the combination of a molecule having the formula BMR-L″-FG2 with a structure having the formula FG1-L′-BIOM to form a structure having the formula BMR-L-BIOM that comprises an optical sensor wherein L is comprised of L′, L″ and FG1 and FG2. In one embodiment FG2 is a cellular receptor distinct from a biomarker receptor in the optical sensor and FG1 is a ligand of the cellular receptor that is distinct for a biomarker for which the optical sensor is intended to detect. In another embodiment FG1 is a cellular receptor distinct from a biomarker receptor in the optical sensor and FG2 is a ligand of the cellular receptor that is distinct for a biomarker for which the optical sensor is intended to detect. In another embodiment a biomarker receptor is immobilized to a biopolymer material using the interaction of avidin and biotin.

For immobilization of a polypeptide to a biopolymer film by indirect covalent attachment, a linker precursor typically used is a heterobifunctional crosslinking agent. Heterobifunctional crosslinking agents are defined as linker precursors having different functional groups that have differing selectivity for a biomarker receptor functional group and a biopolymer material functional group to be combined to form an optical sensor. Examples, by way of illustration and not limitation, of heterobifunctional crosslinking agents are provided in Table 4 shown immediately below.

TABLE 4

| Name | | |
|---|---|---|
| Abbreviation | Structure | Purpose |
| (MAL-PEOx-NHS) | 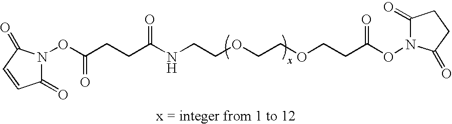 | Crosslinks amine (via NHS) and thio (via maleimide) |
| m-maleimido- benzoyl-N-hydroxy- succinimidyl ester (MBS) | 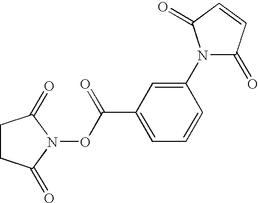 | Crosslinks amine (via NHS) and thio (via maleimide) |
| m-maleimido- benzoyl-N-hydroxy- sulfosuccinimidyl ester (MBS) | 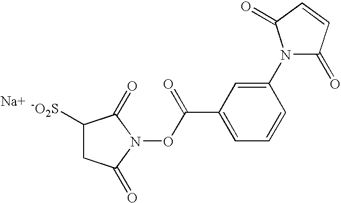 | Crosslinks amine (via NHS) and thio (via maleimide) (water soluble version of MBS) |
| Succinimidyl-4- iodoacetyi-amino- benzoate (SIAB) | 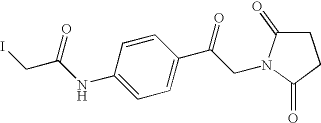 | Crosslinks sulfhydryl (via iodoacetate) and amine (via NHS) |
| Sulfosuccinimidyl-4- iodoacetyl-amino- benzoate (Sulfo- SIAB) | 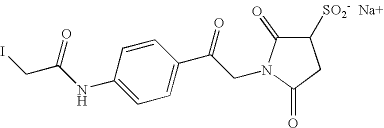 | Crosslinks sulfhydryl (via iodoacetate) and amine (via NHS) (water soluble version of SIAB) |
| N-succinimidyl-3-(2- pyridylthio)- propionate (SPDP) | 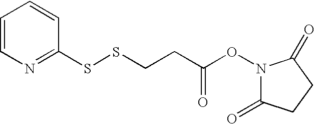 | Crosslinks sulfhydryl (via pyridylthio) and amine (via NHS) |
| N-succinimidyl-6-(3′- (2-pyridyithio)- propionamido)- hexanoate (NHS-Ic- SPDP w/ x = 4) | 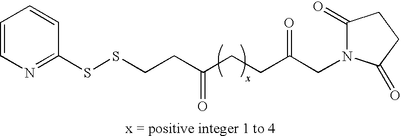 | Crosslinks sulfhydryl (via pyridylthio) and amine (via NHS) |
| Solfosuccinimidyl-6- (3′-(2-pyridylthio)- propionamido)- hexanoate (Sulfo- NHS-Ic-SPDP w/ x = 4) | 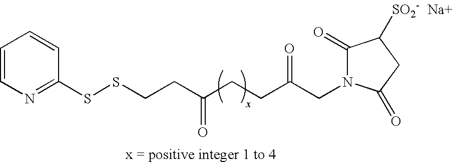 | Crosslinks sulfhydryl (via pyridyithio) and amine (via NHS) (water soluble version of NHS-Ic- SPDP) |
| N-hydroxy- succinimidyl iodoacetate or bromoacetate (NHS-BA or NHS-IA) | 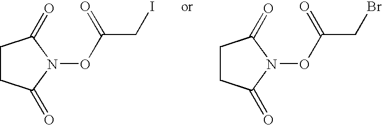 | Crosslinks sulfhydryl (via bromo- or iodo-acetate) and amine via NHS) |
| 2-aminoethyl methane- thiosulfonate HBr (MTSEA) | 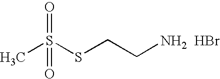 | Crosslinks sulfhydryl (via thiosulfonate) and carboxylic groups (to form an amide |
| Maeimido-propionic acid hydrazide HCl (MPH) x = 3 (MPH) or ε-maleimido- caproic acid hydrazine HCl x = 5 (MCH) or N-(κ- maleimidoundecanoic acid) hydrazide x = 10 (KMUH) | 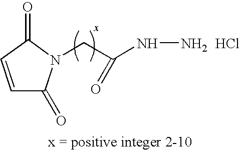 | Crosslinks aldehyde (via hydrazide) and sulfhydryl (via maleimide) |
| 4-(4-N-maleimido- phenylbutyric acid hydrazide HCl (MPBH) | 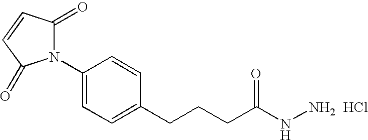 | Crosslinks aldehyde (via hydrazide) and sulfhydryl (via maleimide) |
| 3-(2-pyridylthio)- propionic acid hydrazide HCl (PDPH) | 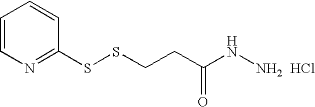 | Crosslinks sulfhydryls (via pyridylthio and aldehyde (via hydrazine) |
| N-(π- maleimidophenyl)- isothiocyanate (MPITC) | 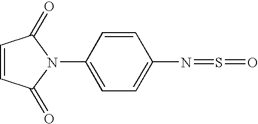 | Crosslinks sulfhydryl (via maleimide) and hydroxyl or amine (via isothiocyanate |

Heterobifunctional crosslinking agents provide the advantage of a stepwise procedure for preparation of a optical sensor comprising a structure having the formula BMR-L-BIOM wherein BMR is a biomarker receptor, L is a linker and BIOM is a biopolymer material through formation of FG1-L-BIOM or a BMR-L-FG2 wherein FG1 represents a first linker precursor functional group and FG2 represents a second linker functional group. Such a stepwise procedure provides greater control over the composition of the optical sensor in contrast to the use of a homobifunctional crosslinking reagent which has identical first and second linker precursor functional groups.

An antibody or antibody fragment is immobilized to a biopolymer material by immobilization attachment means described for polypeptides and in the “Examples” section using a functional group of an amino acid residue comprising the antibody or antibody fragment as the biomarker receptor functional group. Sometimes the amino acid functional group is a sulfhydryl that has been exposed by reduction of a disulfide bridge in an antibody as described in Method 1. In one embodiment an amino acid functional group, such as a ε-amino group of a lysine amino acid residue, is modified to introduce a sulfhydryl group, thus giving a thiolated antibody, examples of which is given in Methods 2 and 3. All methods use an IgG antibody for illustrative purposes and are not meant to be limiting of the inventions described herein.

Method 1 is conducted using the following steps. (1) Prepare a fresh solution of 1 M DTT (15.4 mg/100 μl) in distilled water. (2) IgG solution is concentrated to about 4 mg/ml or higher. The reduction is typically carried out in MES, phosphate or TRIS buffers (pH range 6 to 8). (3) IgG solution is made 20 mM in DTT by adding 20 μl of DTT stock per ml of IgG solution while mixing and is fet stand at room temp for 30 minutes without additional mixing (to minimize reoxidation of cysteines to cystines). (4) The reduced IgG is passed over a filtration column pre-equilibrated with “Exchange Buffer”. Collect 0.25 ml fractions off the column (5) determine the protein concentrations and pool the fractions with the majority of the IgG. This can be done either spectrophotometrically or colorimetrically. Conjugation to a biopolymer material, FG2-L-BIOM wherein FG2 is capable of binding to a sulfhydryl or a crosslinking agent having a maleimide group.

Method 2 is conducted using the following steps. (1) Concentrate the antibody to 5-7 mg/ml, (2) Dissolve 10 mg of N-succinimidyl-5-acetyl-thioacetate (SATA) in 1 ml DMF (SATA:DMF) (3) Add 3.0 μl SATA:DMF per mg of antibody and gently stir solution for 2 hours at room temperature, (4) Dissolve 0.5 g hydroxylamine hydrochloride in 10 ml PBS:EDTA and add 0.25 g NaOH pellets to this solution to neutralize to approximately pH 7.0, (5) Add the hydroxylamine solution to antibody solution at 6.49 μl/μl SATA and gently stir for 30 minutes at room temperature; (6) Equilibrate a desalting column with PBS:EDTA and collect the thiolated antibody in the smallest volume possible without collecting any SATA, (7) Store the thiolated antibody at 2-8° C. for no more than one hour prior to conjugation to a FG2-L-BIOM wherein FG2 is capable of binding to a sulfhydryl or a crosslinking agent having a maleimide group.

A thiolated antibody is alternatively prepared using 2-iminotholane HCl (Traut's reagent) using Method 3 conducted according to the following steps. (1) Dissolve 4 mg antibody in 475 μL of PBS-ETDA buffer at pH 8.0 (coupling buffer), (2) Dissolve 2 mg of Traut's reagent in 1 mL coupling buffer to give a 14.5 mM stock solution, (3) Immediately add 25 μL of the Traut's reagent solution to the antibody solution (results in a 12 fold molar excess of reagent), (4) Purify the thiolated antibody from excess reagent using a desalting column equilibrated with coupling buffer and collect fractions having absorbance at 280 nm.

Sometime an antibody is bound covalently though an aldehyde group obtained from oxidation of carbohydrate moieties in the Fc region of the antibody. Oxidation of carbohydrate on a glycoprotein such as an antibody is accomplished with sodium periodate according to the procedure give in Duan, U.S. Pat. No. 6,218,160 which is incorporated by reference herein in its entirety. A crosslinking agent containing a hydrazine is then used to form a hydrazone which provides a BMR-L-FG2 intermediate.

“Biopolymer immobilization means” as used herein refers to the underlying structure that attaches (i.e., immobilizes) a biopolymer material to a biopolymer support. Attachment may be direct or indirect and through covalent or non-covalent binding as described for immobilization of biomarker receptors. Oftentimes the a lipid biopolymer material is immobilized to a biopolymer support by non-covalent binding through hydrophobic interactions (i.e., van der Waals) with the hydrophobic tails of the crosslinked lipid biopolymer monomers with a hydrophobic surface of the biopolymer support. The hydrophobic support may be present in the support material used in construction of a support, non-limiting examples being the plastic of a microtiter plate. Alternatively, the hydrophobic surface is introduced by chemical modification of a support material in a process referred to as hydrophobization. An example of chemical modification for hydrophobization of a support material to provide a lipid biopolymer support is silylization of glass to introduce a reactive functional group such as an amino group which can then be combined with another reactive group on a hydrophobic molecule such as the amino acid head group of a lipid. Hydrophobization of glass is described elsewhere in the specification while modification of glass and other support materials with polymers is described in U.S. Pat. No. 4,363,634 (Schall) which is incorporated by reference herein in its entirety.

An “OP-polypeptide conjugate” or an “OP covalent adduct” as used here is a molecule comprising a polypeptide having a nucleophile that is covalently modified by a phosphorous atom (i.e., a atom, such as N or O, of the polypeptide-based nucleophile is covalently attached directly to the phosphorous atom) present in an organophosphate compound. When the nucleophile is the hydroxyl group of an active site serine residue in a serine hydrolase, a cholinesterase or an acetylcholinesterase the OP-polypeptide conjugate is referred to as a serine hydrolase OP-conjugate, a cholinesterase OP-conjugate or an acetylcholinesterase OP-conjugate.

An “optical sensor” as used here is a composition having a biopolymer material and a biomarker receptor wherein the receptor is non-releasably immobilized (i.e. attached) either covalently or non-covalently (i.e., covalent or non-covalent attachment) an either directly or optionally through a linker (i.e., immobilization by direct or indirect covalent attachment) to the biopolymer material. Thus, an optical sensor is the minimal arrangement of elements that permits detection of a change in optical property of a biopolymer material upon binging of a biomarker to a biomarker receptor immobilized onto the biopolymer material.

Optical sensor module is a composition that comprises an optical sensor and a biopolymer support or substrate to which the optical sensor is immobilized or is a composition comprising an optical sensor immobilized in the form of a vesicle or liposome. Thus, an optical sensor module may be immobilized onto a rigid biopolymer support material that permits physical handling of the optical sensor module or may be immobilized onto a flexible support such as a vesicle or liposome which permits methods of liquid handling of the optical sensor module. One such method is the liquid transfer of a vesicle or liposome into a well of a microtiter plate or transfer to or through a microfluidic channel of a microfluidic module or the transfer to or through an absorbance flow cell or a fluorescence flow cell.

“Optical property” is as used here is a characteristic of light energy resulting from its interaction with matter. Optical property includes by way of example and not limitation, reflectance, transmission, emission, absorbance, polarization, fluorescence and phosphorescence.

“Detectable change” as used in reference to an optical property refers to a change in presence of an optical property of matter due to its interaction with incident light energy. The change may be the production of an optical property in a substance not present prior to the substance accepting incident light or a change in intensity or wavelength or wavelength distribution of an optical property that was present in a substance previous to its acceptance of incident light energy.

“Colorimetric optical property” refers to a change in color, either in intensity or wavelength (i.e., color) or the production of a color in a substance that may be observed by the human eye under ambient lighting.

“Fluorescent optical property” as used here refers to an optical property of a substance that is associated with fluorescence including but not limited to fluorescence emission or fluorescence emission spectrum, fluorescence absorption spectrum, fluorescence polarization or fluorescence lifetime.

“Transparent” as used herein is a property of a material that permits transmission of incident light energy wherein the transmitted light energy has the same or narrower wavelength range as the incident light energy and wherein the intensity of the light energy transmitted at a given wavelength is a significant fraction of the intensity of the same wavelength in the incident light energy. The significant fraction of light energy transmitted is in a range of between about 10-100%, 20-100%, 30-100%, 40-100%, 50-100%, 60-100%, 70-100%, 80-100% or 90-100% of the incident light energy. A material that is transparent to a narrower wavelength than present in the incident light energy is referred to as a filter. The incident light energy may arise from a light energy source of a biosensor device or a hand help lamp and the material transmitting the incident light is a cover of a optical sensor module or the incident light energy may be the optical energy emitted by a biopolymer material of a optical sensor module having bound biomarker wherein the biopolymer material has been excited by a light energy source from a biosensor device or a hand held lamp.

Simultaneously or near simultaneously refers to the temporal relationship between a cause and detection of a resulting effect, such as irradiation of a biopolymer material and detection of an optical change in the biopolymer material due its interaction with incident light energy, in an elapsed time of sufficiently short duration to allow for detection of the effect. Typically the time delay for detection is determined by the response time of the electronic employed in the detection. For detection of fluorescence emission a maximum elapsed time consistent with near simultaneous detection will depend on the lifetime of the fluorescence.

A “serine hydrolase” includes serine proteases like trypsin, lipases like pancreatic lipase, hormone sensitive lipase, and triacylglycerol lipaseesterases, acetylcholinesterase, thioesterases, certain phospholipases like phospholipase A2 and some amidases like fatty acid amide hydrolase. All serine hydrolases share a catalytic mechanism comprising a serine nucleophile and mechanistically have in common formation of a covalent adduct involving a serine hydroxyl and a moiety derived from a hydrolase substrate. Biomarkers derived from a suicide inhibitor of a serine hydrolase are referred to as serine hydrolase biomarkers Serine hydrolases also include serine endopeptidases under EC classification number 3.4.21 and carboxylic ester hydrolases under EC classification number of 3.1.1. Carboxylic ester hydrolases includes carboxylesterase (3.1.1.1), acetylcholinesterase (3.1.1.7) and cholinesterase (3.1.1.8) also known as butryl cholinesterase. A serine hydrolase used in practicing various embodiments of the invention when isolated is typically from a mammal such as a rodent (e.g mouse or rat), dog, non-human primate or human although a serine hydrolayse isolated from a lower organism may be suitable if it is of similar homology to be predictive of the activity of a serine hydrolase in a higher organism towards a given suicide inhibitor for which it serves as a model.

“Choline esterase” as used herein is a term, unless otherwise specified as butryl cholinesterase or by EC number 3.1.1.8, includes butyryl cholinesterase, acetylcholinesterase and carboxylesterase or is a fragment thereof wherein the fragment substantially retains the catalytic activity of the intact enzyme or has an activity capable of suicide inactivation.

“Introduction opening” and “removal opening” as used here means an orifice through which a liquid may be introduced into contact or removed from contact with an optical sensor that comprises an optical sensor module. Sometimes the introduction and removal openings are discrete orifices as in an absorbance or fluorescence flow cell. Sometime a single orifice serves both purposes in an optical sensor module, which may or may not be sealed by a needle pierceable membrane or septum, and is typically the case for a cuvette, vial or a well of a microtiter plate that functions as a biopolymer support. Sometimes the liquid is a biological liquid containing or suspected of containing a biomarker to which an optical sensor is sensitive or is an aqueous buffer which may serve as a washing fluid for an optical sensor.

An “optical sensor cover” is an optional component of an optical sensor module used in conjunction with a biopolymer support to forms a liquid tight seal, either directly or though an intervening material, that encases an optical sensor that is immobilized to the biopolymer support, with the exception of aforementioned introduction and removal openings incorporated into the optical sensor module which may be optionally sealed with a needle pierceable membrane or septum. Sometimes the optical sensor cover and biopolymer support is comprised of a single contiguous material as is oftentimes the case when a optical sensor is immobilized to a surface of a cuvette or to the inside bottom of a well in a microtiter plate. Another example of an optical sensor module that incorporates an optical sensor cover is a badge that can be carried or worn by an individual working in an environment in which exposure to a chemical insult or environmental toxin such as an organophosphate compound is expected. In such an optical sensor module, a liquid containing a susceptible polypeptide will be in contact with the optical sensor in a sealed enclosure and the optical sensor cover will permit diffusion of the suspected chemical insult in its gas form into the enclosure for dissolution into the fluid so the suspected chemical insult so introduced into the badge will interact with the polypeptide to form a biomarker which is then detected by the optical sensor.

Array or “patterned array” as used here refers to an arrangement of elements (i.e., entities) such as an arrangement of optical sensors or optical sensor module in relationship to a material such as a biopolymer support material or device. In one embodiment, an arrangement of several discrete biopolymer supports having different biomarker receptors immobilized thereto constitutes an array. Such an array allows for the detection of several different biomarkers simultaneously or near simultaneously or sequentially by interrogating each element of the array at once or in sequence with optical energy having the same or different wavelengths and-or intensities. In another embodiment an array of optical sensors is an arrangement of several discrete biopolymer supports having a different density of the same biomarker immobilized thereto or is a contiguous biopolymer support onto which is patterned the same biomarker receptor at different densities. Thus, interrogation of the entire array with the same wavelength and intensity of optical energy will provide a differing pattern of optical change that is indicative of the concentration of biomarker to which the array is exposed. The pattern of differing densities results in the ability to distinguish different concentrations due to elements in the array having lower densities of biomarker receptors becoming saturated (and thus giving maximum optical change sooner than elements having higher densities of biomarker receptors. When an array of such differing densities is incorporated into a badge as previously described an optical sensor module is obtained that is capable of reporting cumulative exposure of an individual to a chemical insult or environmental toxin.

In one embodiment an array is created in a microtiter plate having biopolymer material immobilized to all of the wells and different biomarker receptors or different concentrations or amounts of the same biomarker receptor are then deposited by pipette or liquid handler to some or all of the wells. Reagent to initiate immobilization of the biomarker receptors is then added to the well to which biomarker receptor has been added. Thus, an array of optical sensors (from the viewpoint of the entire microtiter plate) or an array of optical sensor modules (from the viewpoint of the individual wells) is produced and wells to which no biomarker receptor has been added will serve as negative control wells. In another embodiment an array is created by adhering to a contiguous optical sensor or biopolymer material a crisscross pattern of another material which may be the same or different to the material of the optical sensor support such that a liquid tight seal is formed to provide discrete isolated areas of optical sensor or biopolymer material. In the variant where a biopolymer material was segmented (as opposed to the variant where a contiguous optical sensor was segmented), biomarker receptor is the delivered and immobilized, as described for a microtiter plate based array, to each area or “well” to provide an array wherein each element of the array is capable of detecting the same or different biomarker.

It is also contemplated that other arrays will be used with the present invention, including such easily understood patterns as a “+” sign to indicate that presence of a particular biomarker. It is not intended that the present invention be limited to any particular array design or configuration. In still other embodiments, an array is comprised of a PDA-biopolymer film and the fluorescence response allows sampling of multiple biomarker in a single sample of a biological fluid. For example, an array of 100 by 100 milli-micron elements (i.e. section of PDA biopolymer film may be used to create an optical sensor module capable of providing a minimum dynamic range of 14 bits (0-16,000) on up to a range of 17 bits (0-128,000). A 10×10 array of these 1,000 sq. milli micron elements can be manufactured by molecular film deposition techniques that would permit evaluation 100 different samples of biological origin suspected of containing a biomarker such as acetylcholinesterase covalently modified by an organophosphate compound. Thus, in another embodiment of an optical sensor array, one 2.5×7.5 cm glass slide is used to create an array of 10,000 elements of 100×100 milli-micron dimension for screening of biological samples for biomarkers.

“Linear array” as used here means a linear arrangement of elements such as a linear arrangement of optical sensors or optical sensor modules. The linear arrangement may be spatial wherein a one dimensional series of optical sensor elements is present having the same biomarker receptor but present at different densities to provide the equivalent of a “pH stick” for detection of a biomarker. The linear arrangement may be temporal wherein a series of optical sensor modules is presented to a light source of a biosensor device in sequence (e.g. one at a time). The collection of optical sensor modules so presented may be held in a holder contained within a biosensor device, such as a carousel. The carousel is rotated in order to present each optical sensor module in a timed sequence that permits detection of a detectable change in optical property for each optical sensor module upon a coordinated exposure of by incident energy directed by the biosensor device to each optical sensor.

“Machine addressable” or “machine readable array” as used here means an arrangement of elements ordered in a manner suitable for addressing by a machine (e.g. for delivery of a biological fluid to an optical sensor film). Typically, a machine readable array will have a pattern and footprint commonly employed in high throughput screening or combinatorial synthesis, such as found in various microtiter plate formats to simplify software instructions required to address each element of the array.

A “biological sample” as used herein refers to a material from an organism that contains or is suspected to contain a biomarker. Biological samples include blood, serum, plasma, urine, saliva, cerebrospinal fluid, feces, tissue biopsies and the like. Typically, the biological sample will be a liquid biological sample or a liquid extract thereof or a liquid extract of a solid biological sample that is readily manipulated by a hand pipette or a automatic liquid handler. Sometimes a liquid biological sample or extract is diluted with buffer or is passed through a size exclusion or other chromatographic medium to remove components which interfere with detection of a biomarker or to concentrate the sample in suspected biomarker in order to improve sensitivity of detection

“Biosensor device” as used herein refers to any apparatus that incorporates an optical sensor or an optical sensor module (e.g., a microtiter plate array, badges and the like) or is adapted for use with the same (such as a flourescence biosensor device. In one embodiment a biosensor device adapted for use with an optical sensor module comprises a source of incident optical energy, such as a lamp or a laser, for interaction with an optical sensor in the optical sensor module and a detection module such as a fluorescence detector, a photomultiplier, a photon counter or a charge couple device for detection of a change in optical property of a biopolymer material resulting from interaction of a biomarker with a biomarker receptor immobilized onto the biopolymer material and incident optical energy. Also contemplated are biosensor devices as described above further comprising, where appropriate, a microfluidic module or a liquid handler module for handling or movement of liquid samples of biological origin to and from the optical sensor module.

Exposure to OP compounds causes the well-known neurotoxicity resulting from AChE inhibition. AChE inhibition proceeds by the reaction between the critical serine hydroxyl group (Ser-OH) on AChE with the OP compound which a substituted phospho-serine residue. The resultant protein, termed an OP-AChE conjugate, is generally stable which renders AChE inactive and no longer able to hydrolyze the neurotransmitter acetylcholine. In cases where the OP-AChE conjugate is sufficiently stable, surplus acetylcholine reaches toxic concentrations in the neural synapse and can cause a number of neurological maladies (sometimes leading to death) typically starting with nausea, weakness, mild tremor and dizziness. Excessive OP compound exposure has been linked to ataxia, delayed neuropathy, pulmonary toxicity, genotoxicity, Parkinson's and vision loss, although the exact connection between AChE inhibition and any specific disease state still remains unclear.

Several possible biochemical events (shown in Equation 1) may occur after the initial inactivation (i.e. suicide inhibition) of AChE by an OP. The initial covalent adduct formed is an example of a primary organophosphate biomarker. AChE inhibited by an OP compound may reactivate (k₃) via cleavage of the phospho-O-serine bond either spontaneously (water) or mediated by oxime antidotes such as 2-PAM and TMB-4. Reactivation results in restoration of AChE activity once the covalent OP-modification is removed. Another possible pathway that OP-AChE conjugate can undergo is aging in which a group other than the phospho-O-serine bond is cleaved (k₄). The resultant ‘aged’ OP-AChE conjugate is completely unreactive towards antidotes and is considered irreversibly inhibited and is an example of a secondary organophosphate biomarker. An OP compound bears three groups, X, Y and Z (wherein Z is a leaving group) which provides a structure of a resulting OP-AChE conjugate that is highly specific for the OP compound. Overall, the AChE loses a proton in the inhibition, but the gain in covalent modification from the OP is unique and is directly related to the structure of the OP compound.

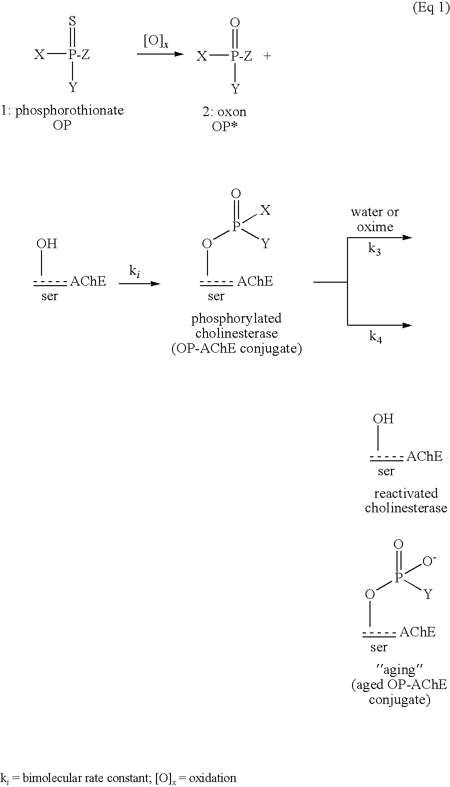

An important toxicological consequences of OP exposure is the near instantaneous reaction of an OP compound with AChE and the ensuing rapid onset of cholinergic symptoms. The rapid formation of the resulting OP-AChE conjugate, post-inhibition pathways and resultant pathology are all related to the structure of the OP agent. Thus, the OP compound and its corresponding OP-AChE conjugate structures play an important role in the duration of toxicity the organism experiences. For example, dimethyl phosphorylated cholinesterase (X=Y=OMe) reactivates readily with a t_{1/2}=4 hrs (human RBC AChE) whereas the diethyl analog (X=Y=OEt) takes more than 36 hrs to reactivate (human RBC AChE) (Wilson, 1992). The difference in structure is relatively small, yet the outcome for the organism is stark-recovery versus acute neurotoxicity. Overall, a rapid reaction to form an OP-AChE conjugate that undergoes a corresponding slow reactivation reaction could be fatal for the organism. Conversely, slow formation of an OP-AChE conjugate and rapid reactivation is less harmful. Therefore, a detection system to assess the level, type and structure of the OP-AChE conjugate and its aged product is of use to determine proper therapeutic intervention. These considerations also magnify the importance of developing methods that distinguish between very precise molecular changes because subtle changes result in marked differences in toxic outcome. It is therefore valuable to devise detection devices and methods that could distinguish between “native” AChE, initial OP-inhibited AChE, and aged OP-AChE conjugates to assess the type and level of exposure from a given OP compound. The term “native” refers to an amino acid sequence that is naturally present in a biological-derived protein and does not refer or imply tertiary structure of that sequence unless indicated otherwise. Although the mechanism of OP action has been known for decades and OP-AChE conjugates have been identified, it is believed no method or device to identify the OP-AChE conjugates based on mechanism has been advanced prior to the instant disclosure.

Several possible biochemical events (shown in Equation 1) may occur after the initial inhibition of AChE by an OP. AChE inhibited by an OP compound may reactivate (k₃) via cleavage of the phospho-O-serine bond either spontaneously (water) or mediated by oxime antidotes such as 2-PAM and TMB-4. Reactivation results in restoration of AChE activity once the covalent OP-modification is removed. Another possible pathway that OP-AChE conjugate can undergo is aging in which a group other than the phospho-O-serine bond is cleaved (k₄). The resultant ‘aged’ OP-AChE conjugate is completely unreactive towards antidotes and is considered irreversibly inhibited. An OP compound bears three groups, X, Y and Z (wherein Z is a leaving group) which provides a structure of a resulting OP-AChE conjugate that is highly specific for the OP compound. Overall, the AChE loses a proton in the inhibition, but the gain in covalent modification from the OP is unique and is directly related to the structure of the OP compound.

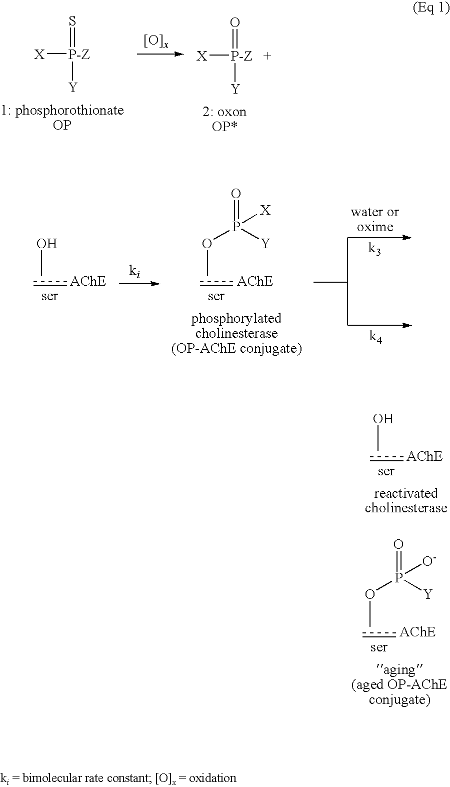

A biomarker initially formed from suicide inactivation of a serine hydrolase an organophosphate compound (i.e. a primary organophosphate biomarker) will have a specific structure (identity of substituents) and arrangement (stereochemistry) of substituents within the OP-protein conjugate that will determine the nature, rate and the outcome of post-inhibitory reactions that modify the primary biomarker to give a secondary biomarker. Oftentimes the secondary biomarker formed subsequent to organophosphate exposure of a serine hydrolase will be an “aged conjugate” as (described in equation 1) and both the primary and secondary organophosphate biomarker are present in a proportion depending on structure of the organophosphate compound and the time since exposure. Thus, in some embodiments each OP compound deposits a distinctive fingerprint on the serine hydrolase, which are the identities and proportions of the primary and secondary organophosphate biomarker that can be specifically identified.

Exposure to, and toxicological assessment following exposure to OP agents is typically determined with a blood “cholinesterase test.” The blood cholinesterase test (BCT) is an assay (known in the art as the Ellman assay) in which cholinesterase activity in the plasma (or serum) and/or red blood cell is measured colorimetrically (Ellman, 1961). For plasma, butyryl-cholinesterase (BuChE) readings are helpful for detecting the early, acute effects of OP poisoning while for red blood cells, AChE readings are somewhat useful in this regard but less sensitive (Padilla, 1995). The BCT is also used to monitor the restoration of AChE activity after exposure to an OP. Through the action of oximes, other therapies or the passage of time (protein synthesis, etc.), enzyme activity can be recovered and the recovered activity monitored by the BCT. Although the BCT has been in use for decades to evaluate exposure to OP insecticides, it is an activity-based assay rather than a direct measurement (molecular species analysis) and is seriously limited with several problems including 1) a pre-exposure baseline cholinesterase value is required to assess the change in activity associated with an OP exposure and pre-screening personnel for this activity is not practical, 2) a reduction in blood cholinesterase activity does not necessarily correlate with the OP-conjugate formed or the resultant neurotoxicity, 3) the activity measurement is only good the for day of exposure since broad statistical dispersion after 24 h renders the test inaccurate, 4) the BCT does not quantify OP exposure, 5) the BCT assay is non-specific—reductions in AChE activity can occur for reasons other than OP exposure (stress, anemia, prescription drugs, etc., 6) although the recovery of enzyme activity can be monitored, the outcome of the remaining inhibited AChE remains unknown, and 7) the assay is ineffectual for chronic exposure, patterns of exposures and/or synergistic interactions.

In line with these concerns, the EPA Office of Pesticide Programs (OPP) released a Science Policy on the Use of Cholinesterase Inhibition for Risk Assessments of Organophosphate and Carbamate Pesticides (1998) that questions the merit of the BCT for determining OP pesticide exposure and toxicity and suggested the need to examine true biomarkers of exposure. Therefore, there is a need for a more specific means for determining exposure to OP compounds which the present disclosure addresses.

Immunochemical methods of analysis have been reported for direct OP analysis (see e.g. Jones, 1995; Edward, 1993; McAdam, 1992, 1993; and reviewed in: Skerritt, 1996; Lucas, 1995; Van Emon, 1990) and used to evaluate the presence of OP compounds in environmental and biological matrices. Antibodies specific to the parent OP structure and their metabolites or fragments of OP structures have been used to estimate the OP concentration but not the products of OP action themselves, namely, the OP-AChE conjugates. Unfortunately, OP compounds are reactive and direct OP compound detection for quantification using an antibody is inherently flawed because the OP compound as an analyte is anticipated to react rapidly with biomolecules, including the antibody used for detection, which reduces or destroys the ability of the antibody to recognize the OP-specific conjugates. Therefore, previously described OP-based antibody detection methods, although of wide interest, currently fall short of the needs for correlating OP compound exposure with toxicity. Moreover, because OP compounds differ in structure, there are a number of possible OP-protein biomarkers that can result from exposure for any given OP compound. As a result, all previously described general assay methods are unable to adequately assess the large number of possible chemical exposures from OP-based agricultural pesticides. This deficiency is removed by the new, comprehensive method for detecting, identifying and quantifying OP exposure as described herein. In this disclosure, we also describe an optical biosensor device, that measures native AChE, OP-modified AChE, and aged OP-AChE conjugates by direct quantitative measurements of such OP-AChE biomarkers. The device is extendable to an array format which allows for an automated sequential or simultaneous analysis of these species. Preparation of a lipid-based polydiacetylene (PDA) polymer is shown in Equation 2.

The di-acetylenic moieties of the monomer molecules are cross-linked by UV irradiation to form the corresponding polydiacetylene polymer (Day, 1978). The cross-linking proceeds through a 1,4 radical polymerization reaction.

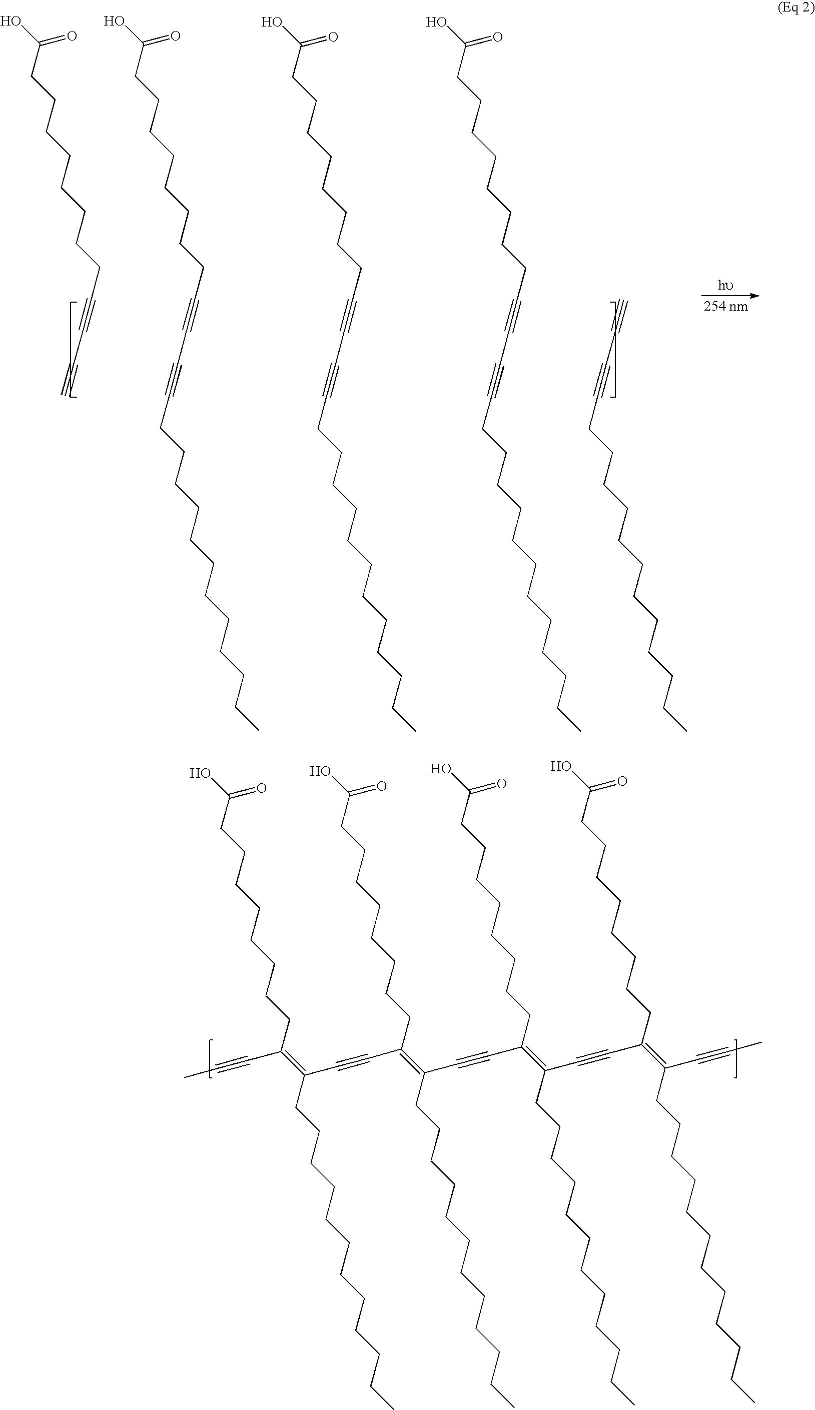

The resulting conjugated PDA polymer backbone imparts a deep blue color to the material due to its broad optical absorbance at approximately 630 nm. Di-acetylenic lipids have previously been shown to form multi-layer polymer films at air-water surfaces (Langmuir-Blodgett films: Day, 1978; Tieke, 1982), as well as tubular (Tieke, 1982) and vesicular structures (Day, 1978; Tieke, 1982). As single crystals or Langmuir-Blodgett films, PDAs have been shown to undergo color transitions from a blue phase to a red phase. These color transitions are produced by external perturbations such as heat (thermochromism: Chance, 1979) or mechanical stress (mechanochromism: Nallicheri, 1991) and are due to changes in the polymer morphology. While the phenomenon of optical transition in PDA-polymers has been extensively studied, the mechanism is unclear. But, it is generally accepted the optical transition is due to conformation change in the PDA side chains and/or disruption in the effective conjugation length of the “ene-yne” backbone (Lio, 1997).

The PDA polymers are characterized by unique properties with respect to their color (chromogenic) or fluorescence (fluorogenic) states. In one example, a lipid-based PDA polymer film has been demonstrated to undergo a chromogenic transition in response to binding of viral receptors to carbohydrate ligands which had been attached to the surface of the PDA polymer film.

Preparation of the carbohydrate-modified PDA polymer film was accomplished by attaching a viral carbohydrate ligand to the hydrophilic terminus of the respective monomer followed by UV-induced polymerization (Charych, 1993; Reichert, 1995). Upon binding of the carbohydrate ligands of the modified PDA polymer film to viral receptors a blue to red transition occurred. In essence, the conjugated backbone of the PDA polymer film was transformed from one electronic state to another upon viral receptor binding to produce a chromomeric optical change. The response of the carbohydrate-modified PDA polymer film was found to be both sensitive to the viral analyte, <80 HAUs: (hemagglutinating units), and quantifiable by measuring the degree of red to blue conversion as a function of virus concentration (Charych, 1993). The response was completely inhibited by adding non-conjugated carbohydrate ligand. The incorporation into the PDA film of an irrelevant carbohydrate in regards to viral binding did not result in a chromomeric response upon exposure to virus. Likewise, exposure of the PDA biopolymer film to high levels of other proteins, such as BSA, gave essentially no response.

Other investigators have demonstrated that antibodies (Abs) conjugated to PDA polymers give a robust chromogenic (blue to red) response (Gill, 2003). IgG antibodies including anti-human α-fetoprotein, anti-E. coli β-galactosidase, anti-BSA and anti-yeast alkaline phosphatases when conjugated to PDA polymers provided blue to red responses upon binding of ligand to antibody. Abs immobilized onto PDA polymer films are referred to as Ab-PDA biopolymer films.

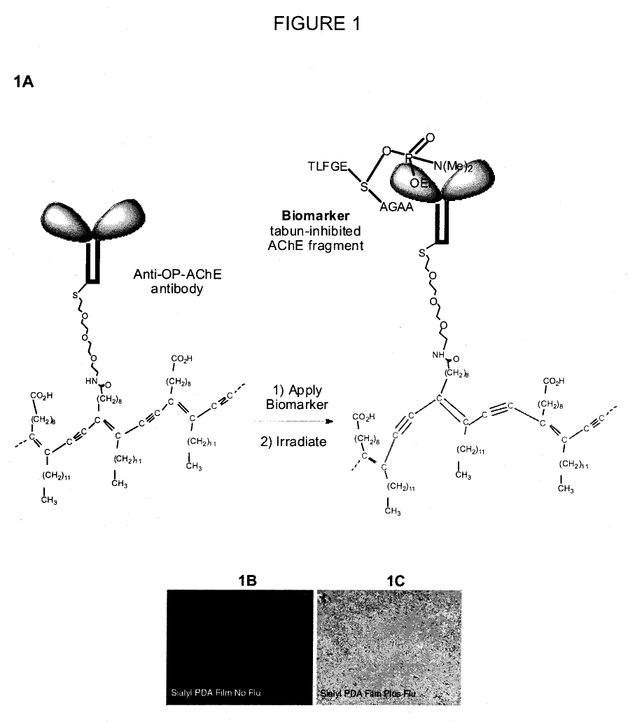

Carbohydrate-modified PDA polymer films were also found to undergo fluorescence emission upon viral binding (Moronne, 2003). Sialic acid, a small-molecule ligand for hemagglutinin surface protein, which is an influenza viral receptor, was covalently attached to lipid-PDA monomers as shown in FIG. 1A. Polymerization of the modified lipid PDA monomers provided the non-fluorescent, PDA polymer film of FIG. 1B. Upon exposure to an influenza virus, the modified PDA polymer film converted to the intensely fluorescent state shown in FIG. 1C. The fluorescence change induced by virus binding to the carbohydrate ligand conjugated to the surface of the PDA polymer film required no additional reagents. This process where no sample preparation is required is referred to as direct detection.

Fluorescence provides a significant advantage over the absorption mode of quantification. In FIG. 1C an intense fluorescence signal is being compared to a virtually zero non-fluorescent signal represented by FIG. 1B. Thus, a fluorogenic method of detection provides an increased signal to noise ratio compared to a chromogenic method of detection. Furthermore, the transition from a non-fluorescent state to a fluorescent state upon binding of an analyte to a surface-modified PDA polymer is rapid (less than a minute) and is resistant to photo-bleaching, unlike other methods of detection which rely upon fluorescent dyes.

Described herein is a biosensor which is designed for detecting relevant proteins, including human proteins that have been altered by organophosphate (OP) compound exposure. In one embodiment a novel biosensor device capable of testing blood, saliva, or other biological fluids or materials, detects and quantifies acute or chronic exposure of an OP compound in a mammal. In one embodiment, a biosensor device is based on a receptor-modified PDA polymer that is adapted for use in the device. In another embodiment a biosensor device is comprised of at least one OP-sensor module, wherein each OP-sensor module is comprised of the same or a different receptor-modified PDA polymer, and one or more modules including an optical sensor module, a processor circuitry module and a microfluidic module. In an embodiment of an OP-sensor module one receptor type is immobilized onto a PDA polymer wherein each receptor type is selective for a specific OP-protein conjugate. In another embodiment an OP-sensor module is comprised of a plurality of receptor types wherein each receptor type is selective for a specific and different OP-protein conjugate wherein each receptor-type is immobilized in a non-random fashion onto a PDA polymer. In yet another embodiment the OP-sensor module is comprised of a plurality of receptor-PDA biopolymers wherein each different receptor type within the OP-sensor module has a different specificity for at least two different OP-protein conjugates wherein the receptor-PDA biopolymers are arrayed in a machine readable fashion. In one embodiment the receptor-PDA biopolymer is a film. In another embodiment of a receptor-PDA biopolymer, the receptor is an antibody or fragment thereof which is selective for a specific OP-protein conjugate. In yet another embodiment the receptor of a receptor-PDA biopolymer is selective for an OP-AChE conjugate. In one method of detection of an OP-protein conjugate binding of the OP-protein conjugate to a receptor-PDA biopolymer results in a calorimetric change. In another method of detection of an OP-protein conjugate binding of the OP-protein conjugates converts the polymer domain of a receptor-PDA biopolymer from a non-fluorescent state to a strongly fluorescent state thereby eliminating the requirement for a chemical-enzyme based amplification technique such as ELISA. In one embodiment the OP-protein conjugate detected is an OP-AChE conjugate. In yet another method of detection of an OP-protein conjugate, a biological sample containing an OP-protein conjugate is contacted with a receptor-PDA biopolymer optionally after a purification step.

Knowledge of the state of an AChE after OP compound exposure or the fractional composition of the AChE, including unmodified AChE, initial OP-inhibited AChE (a primary biomarker) and aged OP-AChE conjugate proteins (a secondary biomarker) guides therapeutic intervention. Therefore, one embodiment for a diagnostic test for exposure to an OP compound determines the degree of AChE inhibition using a biosensor device. In another embodiment the fractional composition of the AChE is determined to identify the OP compound and the degree of exposure from that compound. The design of a biosensor device and methods of detection that can distinguish AChE from OP-AChE conjugates and the various fractional compositions of the conjugates through a mechanism-based approach is disclosed.

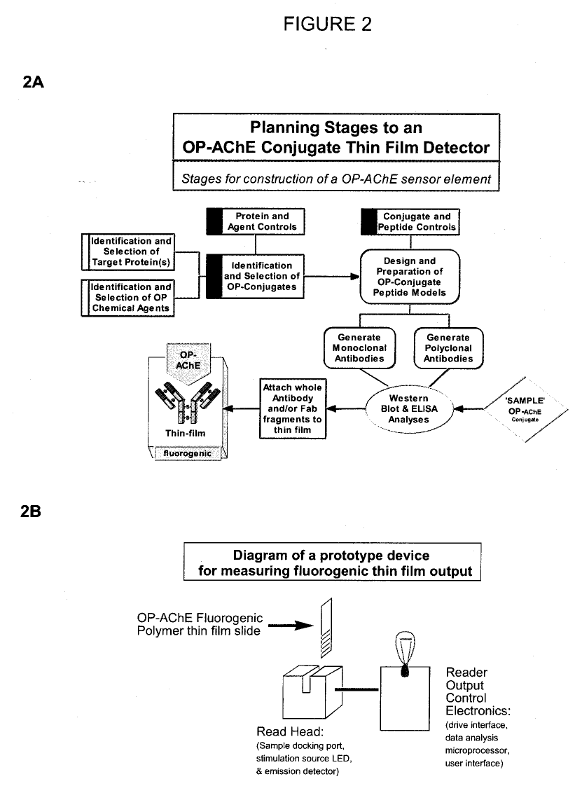

In one embodiment OP-AChE biosensor device is designed in various stages that conceptually follow the strategy depicted in FIG. 2. An Ab-PDA biopolymer is fabricated that can report the binding of unmodified or OP-modified AChE proteins (OP-AChE conjugates) or a combination thereof by a direct optical change in the PDA biopolymer. In a one embodiment, the PDA biopolymer is a film. In an embodiment of a biosensor device, the PDA biopolymer film is adapted for use in an optical detector or reader, and when so adapted is an example of an optical sensor module. In one embodiment a biosensor is comprised of an optical sensor module and a fluorescence detector. The end users of the biosensor device are anticipated to be field personnel and mobile medical units likely to encounter situations where individuals have been exposed to a reactive organophosphoryl compound. Because OP insecticides share a common structure with reactive organophosphoryl compounds, the devices described herein are also readily applied to agricultural exposures to OP compounds.

In one embodiment a color change, based on an induced shift in the absorption spectrum of the receptor-modified PDA polymer upon binding of the OP-AChE conjugate, is monitored using dual wavelength spectrophotometry whereby subtraction of two relatively large signals is used. In another embodiment florescence emission by the receptor-modified PDA polymer, due to a change in fluorescent states upon analyte binding, is monitored whereby a lower background optical signal is subtracted to provide a higher signal to noise ratio.

In one embodiment an OP compound results in a distinct set of OP-AChE conjugates upon reaction with an acetylcholinesterase. The specific structure (identity of substituents) and arrangement (stereochemistry) of the substituents of an OP compound dictates the nature, rate and outcome of the post-inhibitory reaction of the OP compound with an acetylcholinesterase. Thus, the inhibition and post-inhibition reactions represent fractional parts of a population of OP-AChE conjugates from exposure to a given OP compound. For example, sarin (X=Me; Y=OiPr, Z=F) reacts with AChE to form a (iPrO)(Me)P(O)O-serine conjugate (Equation 1). This sarin-AChE conjugate can be rescued with oxime antidotes to restore AChE activity or with time, or a fractional amount of enzyme may spontaneously reactivate. The untreated (iPrO)(Me)P(O)O-serine conjugate can undergo loss of the isopropoxy group to form an aged AChE conjugate containing a (O⁻)(Me)P(O)O-serine residue which is significantly different in chemical properties. Such aged AChE conjugates are refractory to reactivation (oxime therapy is ineffectual) and thus represents irreversible inhibition of AChE. Exposure to an OP compound, such as sarin, therefore, results in three possible states for AChE-inhibited, reactivated, or aged AChE. Sarin is just one example of a reactive organophosphoryl compound that will result in a different set of OP-AChE conjugates.

A detection system to assess the level, type and structure of the OP-AChE conjugates is believed to be described in the present disclosure for the first time. Detection and quantification of the OP-AChE conjugates as described herein identifies an OP compound, such as a reactive organophosphoryl compound, and the amount of exposure from that OP compound, which allows for proper therapeutic intervention and guidance to reduce future exposure events.

Recognition of biomarkers from exposure of a cholinesterase to a reactive inorganic-phosphate compound: The cholinesterases, which are among the principal targets of OP compounds show a large degree of peptide sequence homology in the active site region and share a critical Serine residue known to react with OP compounds. Therefore, when an OP compound reacts with a cholinesterase, specific OP-cholinesterase conjugates are formed wherein the active site serine is modified. To test whether or not antibodies could differentiate between unmodified and OP-modified AChE, two decapeptides were synthesized. Antibodies were produced against decapeptide 3a that contains an unmodified serine hydroxyl (R=H) and the corresponding (unsubstituted) phosphoserine decapeptide 3b. Example abbreviated structures of an AChE active site decapeptide (3a) and a phosphodecapeptide (3b) are given as follows.

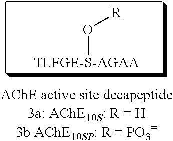

For constructing one embodiment of a biosensor device which detects exposure to an inorganic phosphate compound, polyclonal antibodies (George, 2003) were obtained which recognize a decapeptide 3a derived from the “native” sequence of an acetylcholinesterase (rMoAChE_{10S}). Polyclonal antibodies which recognize the phosphorylated form of the decapeptide 3b resulting from exposure of the AChE to POCl₃ (rMoAChE_{10SP} wherein rMoAChE=mouse recombinant acetylcholinesterase; 10=decapeptide; S=serine-OH; P=inorganic phosphate group) were also obtained. Although the phosphate dianion residue of 3b does not correlate with OP-conjugate structures which result from reaction of a cholinesterase with an OP compound, it does correlate with the product that results from reaction of a cholinesterase with a reactive inorganic phosphate compound such as POCl₃.

It has been shown the anti-AChE_{10S} antibodies react selectively with denatured AChE and do not recognize any other type of AChE which has been modified by phosphorylation, including phosphorylation by POCl₃ (AChE-SerOPO₃⁼) or phosphorylation by OP compounds which give initial AChE OP-modified or aged OP-AChE conjugates. Likewise, anti-AChE_{10SP} reacts selectively with AChE phosphorylated by POCl₃; but does not recognize native AChE nor AChE inhibited by various OP compounds. It has been further shown that recognition of unmodified and POCl₃-modified AChE by these antibodies correlated well with the amount of AChE inhibition and reactivation kinetics for the AChE so modified. In one embodiment a PDA biopolymer incorporates an anti-AChE_{10S} antibody or an AChE_{10SP} antibody to detect exposure of an AChE to an inorganic phosphorylation agent such as POCl₃, and provides a means for time-dependent analysis of exposure the inorganic phosphorylation agent. In another embodiment the anti-AChE_{10S} anti-AChE_{10SP} antibody-based PDA biopolymers are films. In one embodiment, a Ab-PDA biopolymer film is comprised of an anti-AChE_{10S} antibody or an AChE_{10SP} antibody. In another embodiment a combination of an anti-AChE_{10S} antibody and an AChE_{10SP} antibody are incorporated in a non-random or machine addressable array into an OP-sensor module. In another embodiment, recognition fragments of the antibodies, such as Fab fragments, or hyper-variable Fv fragments are used. In one embodiment a biosensor device is used to quantify the degree of exposure to a reactive inorganic phosphate compound. In another embodiment of a biosensor device for quantifying exposure to an inorganic phosphorylation agent, the optical change measured is fluorescence emission.

Recognition of biomarkers from exposure of a cholinesterase to an organo-phosphate compound: Using a mechanism-based rationale to define the location and precise chemical modification caused by an OP compound, specific OP-conjugates can be predicted resulting from modification of a cholinesterase by the OP compound. The OP-conjugates are biomarkers of OP exposure and represent unique chemically modified macromolecular species that are specifically recognized by antibodies raised against protein fragments containing the OP-conjugates. Use of the anti-OP-conjugate antibodies allows for selective detection and differentiation from native macromolecules and other OP-conjugates resulting from other OP compound exposures.

To construct a biosensor device that can readily detect biomarkers, such as an acetylcholinesterase modified by an OP compound, a panel of receptor molecules is selected that can continue functioning upon conjugation to a PDA biopolymer. In one embodiment the receptor molecule is an antibody which recognizes an OP-conjugate. PDA biopolymers comprising antibodies (Abs) that can recognize OP-modified AChE conjugates (OP-AChE conjugates) are an invention of the instant disclosure. Construction of the Ab-based PDA biopolymers requires immobilization of the Abs (or a recognition fragment thereof) by a non-covalent means of attachment which comprises ionic or hydrogen bonding interactions or by a covalent means of attachment either through direct conjugation of the Abs (or recognition fragments thereof) to the PDA polymer or optionally through a linker. Methods are described that will allow Ab-PDA biopolymers to be designed and prepared. In one embodiment an Ab-PDA biopolymer is a film. Methods for designing and preparing an Ab-based PDA biopolymer film to detect OP-AChE conjugates at levels present in human serum are also described. Also described is a biosensor device for detection and quantification of OP compound exposure to a cholinesterase.

In some embodiments an antibody to an OP-cholinesterase or OP-polypeptide conjugate (example of an anti-OP conjugate antibody) is used as a biomarker receptor in a PDA-biopolymer film for detection of an organophosphate biomarker. In one embodiment, a PDA biopolymer film is comprised of an anti-OP-AChE conjugate antibody. In another embodiment a combination of anti-OP-AChE conjugate antibodies are incorporated in a non-random or machine addressable array into an OP-sensor module which is comprised of one or more PDA biopolymer films wherein the array is comprised of at least two anti-OP-AChE conjugate antibody types wherein each antibody type has a different selectivity for at least two different OP-AChE conjugates. In another embodiment, recognition fragments of the antibodies, including Fab fragments and hyper-variable Fv fragments, are used. In one embodiment a biosensor device is used to quantify the degree of exposure to an OP compound. In another embodiment of a biosensor device for quantifying exposure to an OP compound, the optical change measured is fluorescence emission. In another embodiment the biosensor device is used to measure rates of recovery of AChE upon treatment of a subject exposed to an OP compound with an oxime-based antidote.

To obtain the requisite antibody for incorporation as a biomarker receptor into an optical sensor for detection of a organophoshate biomarker resulting from interaction of a organophosphate compound with a cholinesterase, an antigen comprising or consisting of an decapeptide is used wherein the decapeptide contains (1) a serine residue or a serine analog having a phosphorous containing moiety attached thereto and (2) flanking amino acid residues that correspond to the active site serine and flanking amino acid residues in a cholinesterase to be modified by the organophosphate compound. The decapeptide sequence typically corresponds in sequence to a mammalian butyryl cholinesterase, carboxylesterase or acetylcholinesterase. The phosphorous containing moiety will typically be identical in structure to the phosphorous containing moiety deposited on the cholinesterase either initially (i.e. the phosphorous containing moiety of a primary organophosphate biomarker) or after “aging” (i.e. the phosphorous containing moiety of a secondary organophosphate biomarker) to allow for identification of the organophosphate compound that is responsible for the biomarker. In one embodiment for the purpose of eliciting of an anti-OP-cholinesterase conjugate antibody an octapeptide is used having a serine phosphoester residue as the mimic for the active site serine residue present in a cholinesterase that is modified by an organophosphate compound. In other embodiment a serine phosphonate residue is used in place of the serine phosphoester in the octapeptide wherein the serine —O— has been replaced by a carbon atom. Use of such a serine phosphonate antigen may be advantageous if the lifetime of an analogous serine phosphoester is too short to permit production the desired antibody an immune response.

Example amino acid sequences that form the basis of antigens useful for eliciting antibodies to an organophosphate biomarker that results from suicide inactivation of a cholinesterase by an organophosphate compound will have serine residue, which corresponding to the serine in the active site of a cholinesterase, modified to give a phospho monoester phospho diester or will be replaced with an phosphonate analog (i.e., a phosphonoserine) as described immediately above for decapeptide-based antigens. Peptide sequences beyond decapeptide which incorporate addition amino acid residues flanking the serine active site may be used to obtain biomarker receptors (i.e., anti-OP-conjugate antibodies) of increased sensitivity but at the expense of greater synthetic difficult in producing the requisite antigen.

Example peptide sequences to guide the design of an antigen to elicit antibodies for an organophosphate biomarker resulting from interaction of a organophosphate compound with a cholinesterase comprise or consist of TLFGE[S]AGAA (SEQ ID 2), VTLFGE[S]AGAAS (SEQ ID 3), TL FGE[S]AGAAS (SEQ ID 4), TLFGE[S]AGAA (SEQ ID 5), LFGE[S]AGAAS (SEQ ID 6), VTLFGE[S]AGA (SEQ ID 7), VTIFGE[S]AGGES (SEQ ID 8), TIFGE[S]AGGE (SEQ ID 9), TIFGE[S]AGGES (SEQ ID 10), VTIFGE[S]AGGE (SEQ ID 11), IFGE[S]AGGES (SEQ ID 12), VTIFGE[S]AGG (SEQ ID 13) where the serine in brackets indicates the serine mapping to the active site serine of butyryl cholinesterase, acetylcholinesterase or carboxylesterase. Peptide sequences also contemplated as a basis for designing antibody antigens for eliciting anti-OP-conjugate antibodies are the peptides of SEQ 2-13 having 1-6, typically 1-2 conservative amino acid replacements according to Table A for amino acid residues flanking the bracketed serine.

Preparation and use of a receptor-PDA biopolymer for detecting and quantifying exposure to an OP compound by a protein through detection of biomarkers of such exposure is illustrated for acetylcholinesterase and the reactive organophosphoryl compound sarin. Extension of this description for determining exposure from other OP compounds to other proteins which provide biomarkers of such exposures, including other cholinesterases, should be evident to one skilled in the art.

### EXAMPLES

### Example 1

### Identification, Synthesis, Purification and Characterization of OP-Modified Peptide Conjugates Representing Ache Inhibited by Sarin

In the following the section the term “native” refers to an amino acid sequence that is present in biological-derived protein and does not refer to the tertiary structure of a peptide containing the amino acid sequence. To obtain antibodies to sarin-modified AChE, a peptide corresponding in sequence to the active site of an acetylcholinesterase and containing the active site Serine is prepared. Also, peptides containing serine residues modified at the hydroxyl with phosphorus groups, expected from the mechanism by which sarin inhibits AChE, are prepared. The modified peptides correlate in structure to the initially formed OP-AChE conjugate and the subsequently formed aged OP-AChE conjugate. The peptide to be chosen represents a sequence sufficient to generate antibodies to allow for specific identification of the phosphorus group in the context of the polypeptide chain of the OP-modified AChE. In one embodiment the peptides are decapeptides. In another embodiment the peptides are decapeptides characterized by an amino acid sequence wherein the serine residue has four to five amino acid residues flanking its N- and C-termini.

“Native” decapeptide 3a is reacted with N,N-diisopropyl isopropoxy methylphosphonamidite, then oxidized, to give decapeptide 4-sarin (R=iPr) which is analogous to a sarin-AChE conjugate. Reaction with the cyanoethoxy phosphonamidite reagent affords, after oxidation and beta-elimination, the sarin-aged (3a). Reaction conditions are similar for reactions reported with Glu-Ser-Ala tripeptides (Suarez and Thompson, 1999). Reaction progress is monitored using ³¹P NMR and peptide structures are confirmed by QTOF MS-MS analysis (Spaulding, 2006).

The “native”, unmodified decapeptide active site-containing sequence TLFGESAGAA (SEQ ID 2) of Equation 3 is prepared by standard peptide solid phase synthesis. Purification is conducted by reversed-phase preparative HPLC.

### Example 2

### Preparation of Hapten-Carrier Protein for Production of Polyclonal Antibodies that are Specific for “Native” AChE, AChE Phosphopeptide and Sarin-AChE Conjugates

Decapeptides representing the initial VX-inhibited, Sarin-inhibited, and Soman-inhibited AChE OP-conjugates (4-VX, 4-sarin, and 4-soman) are prepared from three distinct methyl phosphonamidite reagents that vary only in the identity of the alkoxy group. Reaction of the decapeptide 3a with each of the methyl phosphonamidites in which the alkoxy group varies from ethoxy (VX), to isopropoxyl (sarin), to isohexyl (soman) followed by oxidation of the resulting product to the oxon affords sarin-inhibited (3-sarin), VX-inhibited (3-VX), and soman-inhibited (3-soman) decapeptide structures, respectively. The methyl phosphonamidite reagents are prepared by sequential reaction of methylphosphinic dichloride (MePCl₂) with an appropriate alkoxide and HN(iPr)₂.

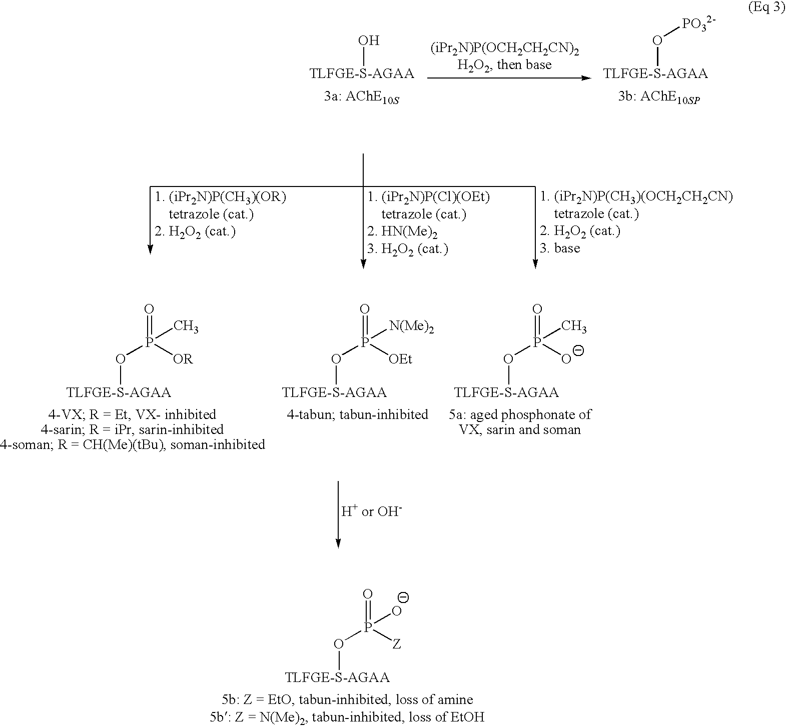

The initial sarin-inhibited AChE undergoes aging to form a methylphosphonate oxyanion due to hydrolytic removal of the alkoxy group. Because of the commonality in structure between sarin, VX and soman, the aged OP-conjugates resulting from exposure of a cholinesterase from any one of these reactive organophosphoryl compounds are identical. Therefore, the hydrolysis of 4-sarin provides 5a, which represents the analogous decapeptide analogs of VX-aged, Sarin-aged, and Soman-aged AChE conjugates. Purification of 4a is by ZipTip or Zn-chelate column chromatography and is well known in the art for isolating phospho-containing peptides.

Antibodies to truncated versions of OP-AChE conjugates that are utilized in the analysis of intact OP-AChE adducts (biomarkers) are described. Production of polyclonal antibodies against “native” decapeptide a, phosphodecapeptide 3b, sarin-modified (4-sarin) and sarin-aged (5a) decapeptides are described for illustrative purposes and is not meant to limit the scope of the inventions disclosed in the instant application. The antibodies to 3a, 3b, 4-sarin and 4a permit detection of sarin-AChE conjugates (initially inhibited and aged), unmodified AChE and phospho-AChE which is not expected to represent a structure resulting from exposure of an cholinesterase to an OP compound.

For production of the polyclonal antibodies decapeptide 3a, phospho-decapeptide 3b, sarin-inhibited 4-sarin and aged sarin decapeptides 4a are each attached to a linker group. The resulting decapeptide-linker intermediates are then conjugated to a carrier protein such as KLH or BSA to form hapten-carrier proteins required for immunization. The ratio of hapten to carrier protein is determined. Decapeptide-linker-KLH conjugates are injected into rabbits to generate the corresponding polyclonal antibodies. Cross-reactivity, specificity and epitope map for polyclonal antibodies obtained from each hapten-carrier protein are determined. The polyclonal antibodies raised against decapeptide 3a will not recognize the aged-OP conjugate from AChE exposure to sarin, soman or VX.

Phosphonate conjugates represent alternative haptens which are identical to phosphate-containing conjugates such as 3a, 3b and 4-sarin except the serine oxygen is replaced by a CH₂ group. The phosphonate conjugates are characterized by a phosphorus-peptide linkage that is stable to hydrolytic and phosphatase cleavage. Preparation of such conjugates is described in Example 7.

The following procedure is used to prepare the requisite hapten-carrier proteins: A linker devoid of secondary structure such as a polyglycine, PEG, or aliphatic group is appended to a decapeptide in order to enhance the immuno-dominance of a desired decapeptide in a hapten-carrier protein. Peptide coupling or condensation procedures known in the art are used to attach the linker to the decapeptide. The resulting decapeptide-linker product is attached to a carrier protein such as KLH or BSA using coupling agents such as a carbodiimide optionally in the presence of N-hydroxysuccinimide (NHS) by standard procedures (Bauminger, 1980; Erlanger, 1980). Purification of the BSA conjugate via molecular exclusion or dialysis is conducted. The number of haptens per protein (hapten carrier protein ratio) is determined spectroscopically or by titration of free lysine residues (Sanger, 1949; Habeeb, 1966). A preferred hapten to carrier protein is in the range of about 15-30. Alternatively, glutaraldehyde is used as a linker to attach a carrier protein to a decapeptide which is functionalized with an amino group.

### Example 3

### Production of Polyclonal Antibodies which Recognize OP-Conjugates

A sufficient amount for each hapten-carrier protein prepared according to Example 2 is emulsified in Freud's Complete Adjuvant (FCA) and is used to immunize rabbits. A standard or typical anti-sera production protocol is shown in Table 1.

TABLE 1
Immunization

| Day | Procedure |
|---|---|
| 0 | NZW Female Rabbit - |
| | Prebleed + Sample - |
| | 1Y SC/D |
| 14 | Boost SC |
| 28 | Boost SC |
| 38 | 1st Production Bleed + |
| | Sample |
| 38* | Optional ELISA |
| | Analysis (1st bleed vs. |
| | pre-bleed) |
| 42 | Boost SC |
| 52 | 2nd Production Bleed + |
| | Sample |
| 52* | Optional ELISA |
| | Analysis (2nd bleed vs. |
| | 1st bleed) |
| 56 | Boost SC |
| 66 | 3rd Production Bleed + |
| | Sample |
| 66* | Optional ELISA |
| | Analysis (3rd bleed vs. |
| | 2nd bleed) |
| 69 | Client Options: |
| | exsanguinate, |
| | terminate, extend |
| | protocol |

### Example 4

### Preparation of Antibody-PDA Biopolymer Films Supported on a Biopolymer Substrate

An example procedure to prepare an Ab-PDA biopolymer film supported on a biopolymer substrate uses the following steps.

(1) Lipid-PDA monomers are synthesized.

(2) A Langmuir-Blodgett film is produced from PDA-forming monomers by polymerization of monomers to give a PDA-polymer film.

(3) Polyclonal antibodies from Example 3 are immobilized onto a lipid PDA monomer or on a lipid PDA polymer film.

(4) An Ab-PDA biopolymer film is adhered to a biopolymer substrate such as a coated glass slide.

(5) The antibody density on an Ab-PDA biopolymer film is determined, for example, by a labeled secondary antibody in an ELISA format.

The order of steps given is not limiting on the methods for preparing Ab-PDA biopolymer films. One method for Ab attachment to a Langmuir-Blodgett film is given by Equation 4.

Procedures for preparing Langmuir-Blodgett films are described in Charych, et al. U.S. Pat. No. 6,395,561 (Filing date Dec. 14, 1999), Charych et al. U.S. Pat. No. 6,001,556 (Filing date Jan. 26, 1996) and Moronne, et al. US Pat. Application Publication No. 2003/0129618 (filing date Jul. 10, 2003) the disclosures of which are incorporated by reference.

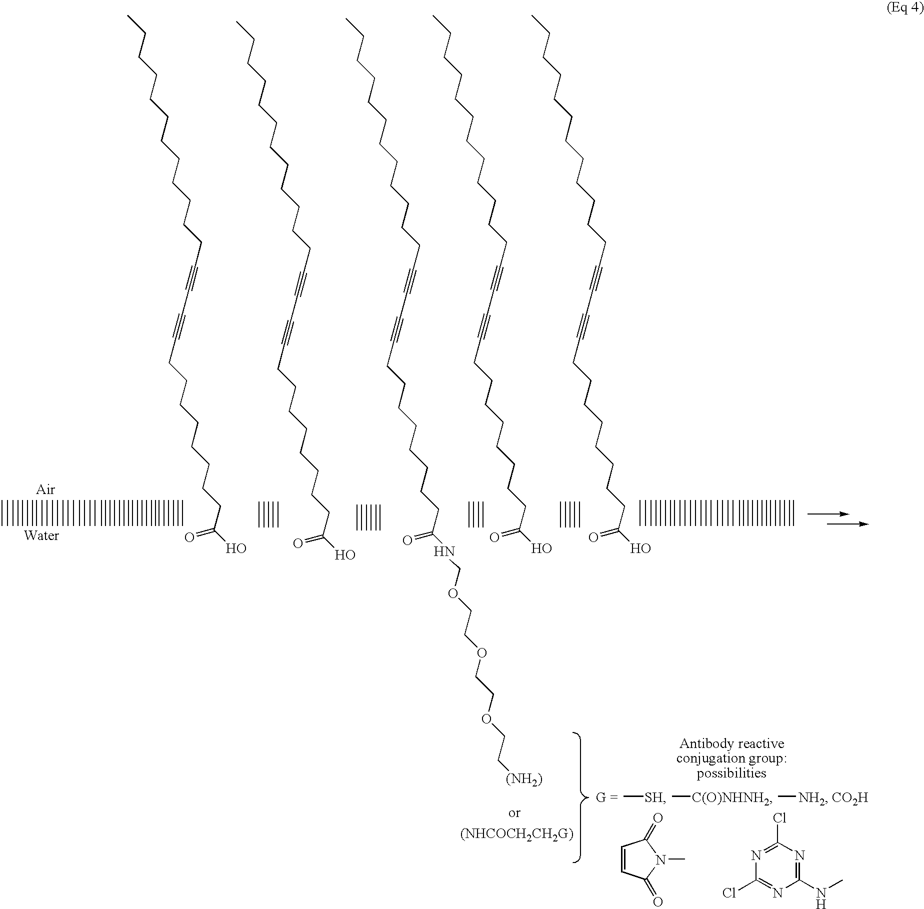

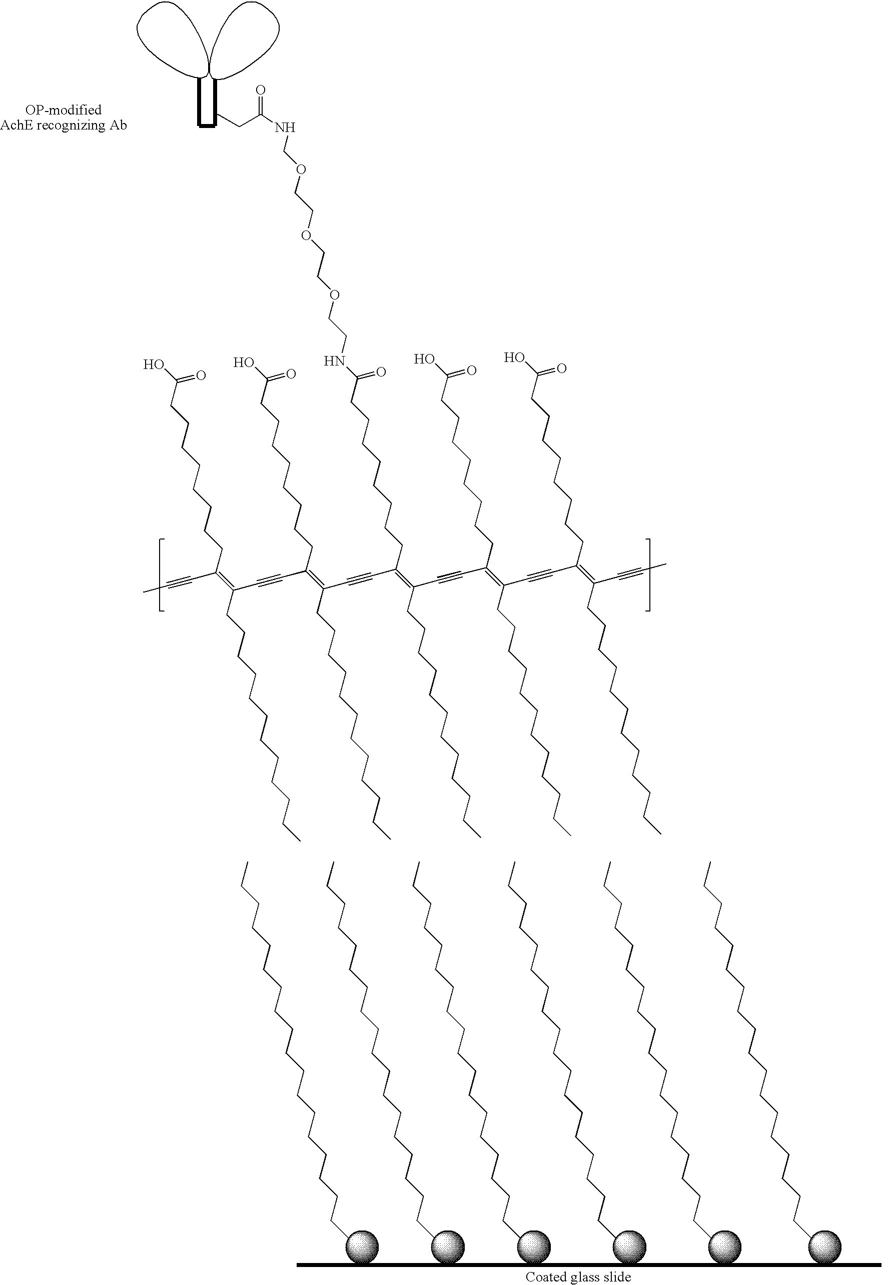

Thin, monolayer films of PDA-forming monomers or Ab-PDA-forming monomers are spread on a water surface in a Langmuir-Blodgett (LB) trough. The monomer mix consists of matrix lipids (10,12 pentacosadiynoic acid: PCDA) and antibody conjugation reactive lipids, such as N-(11-amino-3,6,9-trioxyundecanyl)-10,12-pentacosadiynamide (Spevak, 1993). The monolayer is compressed and polymerized by UV light exposure (Charych, 1993).

The anti-AChE and anti-OP-AChE antibody-modified PDA polymers are tested for level of background fluorescence. The responsiveness of the Ab-PDA biopolymer films are also tested for fluorescence changes upon exposure of the biopolymer film to standardized and doped test solutions with varying levels of AChE and OP-modified AChE proteins. One method for testing responsiveness uses the following steps as exemplified for sarin exposed AChE.

(1) Ab-PDA biopolymer films are exposed to varying concentrations of a decapeptide test sample including “native” decapeptide 3a, phosphodecapeptide 3b, sarin-modified (4-sarin) and sarin-aged (5a) decapeptides to determine fluorogenic responses and establish standard response curves.

(2) Ab-PDA biopolymer films are exposed to varying concentrations of protein test sample including AChE and OP-AChE conjugates to determine fluorogenic responses and establish standard response curves.

(3) Non-AChE derived proteins and decapeptides are spiked into test samples to determine the amounts of false positive responses

Ab-PDA biopolymers films are prepared which are characterized by varying amounts of immobilized antibody, such as anti-AChE antibody or an anti-sarin-AChE conjugate antibody or combinations thereof, and are tested with varying concentrations of peptides such as 3a, 4-sarin and 5a and proteins such as AChE and OP-AChE conjugates. The biopolymer films are inspected under a fluorescent microscope using a standard rhodamine excitation and red LP emission filter set. Detection response curves are generated and an Ab-PDA biopolymer is selected for use in a biosensor device which provides the optimal signal response to a minimal analyte concentration (lowest level of quantification) that is commensurate with the exposure to the OP compound to be analyzed.

One method for preparing PDA-biopolymer films conjugates an Ab to PDA polymer through a linker. The antibody and a bi-functional molecule (which provides the linker), such as N-sulfosuccinimidyl-4-(maleimidomethyl)-cyclohexane-1-carboxylate, is introduced into the water phase and allowed to react (Gill, 2003). Over a time course of several hours, the Ab-PDA biopolymer film is lifted off the Langmuir-Blodgett water surface and the extent of Ab conjugation is determined by exposure of the film to a labeled secondary antibody. The secondary label is chosen so as to be observable by a different fluorescent signal non-overlapping with the signal from possible film fluorescence that may be inducted through polymer Ab-secondary Ab binding.

Another method for preparing PDA-biopolymer films immobilizes an Ab to a PDA-forming monomer by covalent attachment (conjugation). The amounts of PDA-forming monomers in the mix are adjusted to provide varying levels of antibodies immobilized on the PDA biopolymer film. One antibody conjugation procedure uses periodate oxidation of the Fc carbohydrate groups of the Ab to yield aldehyde moieties (O'Shannessy, 1985). A PDA polymer or a PDA-forming monomer which contains hydrazine groups is then reacted with the aldehyde moieties to yield Ab-PDA biopolymer films wherein the Ab is covalently attached (conjugated) directly to the PDA polymer through its Fc region.

For preparation of an OP-sensor module, a PDA biopolymer film is lifted off the Langmuir-Blodgett water surface and is transferred to a coated glass surface so as to avoid mechanical stress that would induce fluorescence and lead to an increase in background noise. Glass microscope slides that have been made hydrophobic are used to lift the non-fluorescent Ab-PDA biopolymer films off the water surface (Charych, 1993). After this procedure, the antibodies are exposed to the slide surface and are analyzed by secondary antibody binding.

In one method of analysis for OP exposure the cholinesterase so exposed is denatured either chemically or thermally before application to the receptor-modified PDA polymer.

### Example 5

### Preparation of OP-Modified Peptides Representing AChE Inhibited by VX, Sarin or Soman

To obtain antibodies to AChE which are modified by the reactive organophosphoryl compounds VX, soman, sarin or tabun, the “native” decapeptide 3a, which contains a serine amino acid residue corresponding to the serine in the active site of the AChE, is reacted with a reactive organophosphoryl precursor to provide modified decapeptides that correlate with the structures resulting from reaction of that same reactive organophosphoryl compound at the active site of AChE. The structures are represented by 4-VX, 4-sarin, 4-soman and 4-tabun. Following inhibition by each agent, AChE undergoes “aging” to form the methylphosphonate 5a (for sarin, soman and VX) or the ethoxy 5b or dimethylamine 5b′ analogs (for tabun). Preparation of the decapeptides corresponding to the OP-AChE conjugates resulting from initial reaction of a reactive organophosphoryl compound (i.e, 4) is as follows:

Native decapeptide 3a is reacted with N,N-diisopropyl ethoxy methylphosphonamidite, and the resulting product is oxidized to give the phosphorylated decapeptide 4-VX (R=Et) which represents the structure from initial AChE inhibition with VX. Native decapeptide 3a is reacted with N,N-diisopropyl isohexyl methylphosphonamidite and the resulting product is oxidized to give the phosphorylated decapeptide 4-soman (where R═CH(Me)t-Bu) which corresponds to the OP-AChE conjugate resulting from initial AChE inhibition with soman. Native decapeptide 3a is reacted with N,N-diisopropyl ethoxy chlorophosphonamidite and the resulting product is treated with dimethylamine followed by oxidation to give 4-tabun which represents the structure from initial AChE inhibition with tabun.

The synthesis of methylphosphonate 5a, corresponding to aged AChE which forms subsequent to initial exposure of AChE to VX, sarin or soman is described in Example 2. Two modified decapeptides 5b (Z=OEt, loss of NMe₂) and 5b′ (Z=NMe₂, loss of EtO) which correspond to aged AChE from tabun exposure are prepared, according to the procedure in Example 6, by base and acid hydrolysis, respectively. Reaction progress is monitored using ³¹P NMR and structures are confirmed by MS-MS and NMR. Analysis by ³¹P NMR affords a unique chemical shift that is distinct for each modified decapeptide. The ³¹P chemical shift of the methyl phosphonate 5a corresponding to aged inhibition of AChE with VX, sarin and soman differs not only from structures 4, which correspond to the OP-AChE conjugates from initial inhibition of AChE with VX or sarin but are also distinct from the structures 5b and 5b′ which corresponds to aged inhibition of AChE with tabun.

Alternatively, modified decapeptides suitable for eliciting antibodies which recognize AChE inhibited by reactive organophosphoryl compounds are prepared using the phosphonate analogs 6, where the oxygen which form the bond from the decapeptide to the phosphorous atom is replaced by a CH₂, or are prepared using thiol analogs 7 (not shown) where the serine residue is replaced by a cysteine (thus, the —S atom from Cys forms the bond between the decapeptide and the phosphorus atom).

### Example 6

### Preparation of OP-Modified Peptides Representing AChE Inhibited by Tabun

Ethoxy, N,N-diisopropyl phosphorochloridite is reacted with the decapeptide 3a followed by displacement of chloride with dimethylamine and subsequent oxidation to afford the decapeptide (4-tabun) which correspond to AChE initially inhibited with tabun.

Loss of the dimethylamine or the ethoxy group from 5a affords structures 5b or 5b′ respectively, which correspond to aged tabun inhibition of AChE. Acid hydrolysis with aqueous chloroacetic acid of 5a results in loss of the dimethyl amine group to provide 5b, whereas base hydrolysis with 1 N NaOH removes the ethoxy group to give 5b′

### Example 7

### Synthesis of Phosphonoserine Analogs of OP-Modified Peptides

Decapeptides in which the easily cleavable serine-O-phosphate ester bond is replaced with a more stable phosphonate linkage (6) will allow for hapten recognition while providing for a longer in vivo lifetime. As shown in FIG. 4, the serine oxygen atom is replaced with a methylene while maintaining the identical peptide sequences at N- and C-termini.

To synthesize a phosphonate peptide analog, the serine residue is replaced with a phosphonate analog as shown in the following.

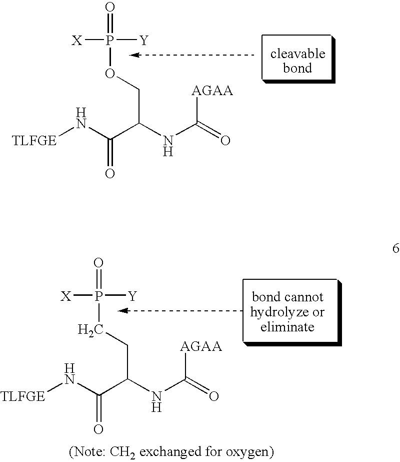

The phosphonate structure substituting for the serine is prepared from the known aminophosphonic acid, AP4. A protected form of AP4 (7) is synthesized according to the procedure of Lohse (1998) (Eq 5). Attachment of the protected AP4 to the N-term and C-term peptide fragments forms decapeptide 7, which is the phosphonate analog of 3a. The peptide fragments are prepared on solid support or in solution phase using standard peptide chemistry techniques.

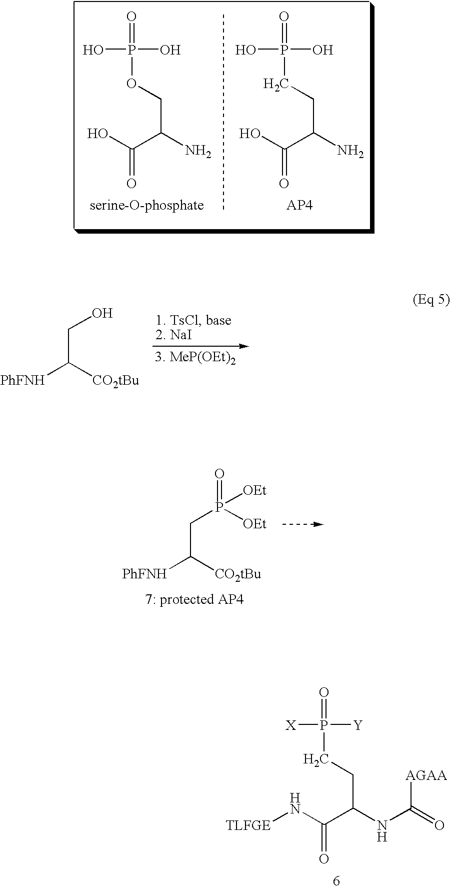

Preparation of 6-VX (X=Me; Y=OEt; Eq 5; phosphono analog of 4-VX) proceeds by conversion of t-butyl, N-phenylfluorenyl serine to its tosylate followed by displacement of the tosyloxy group with iodide. The iodo intermediate is then reacted with diethyl methylphosphite to afford the protected methyl, ethoxyphosphinate serine. Deprotection of the carboxylic ester and coupling to an appropriately protected tetrapeptide with the sequence of AAGA is followed by deprotection of the amine and coupling to an appropriately protected pentapeptide with the sequence of TLFGE to provide 6-VX. The 6-soman and 6-sarin phosphonate analogs are prepared by the same synthesis with a change only in the nature of the Y (alkoxy) group.

### Example 8

### Preparation of Polyclonal Antibodies which Recognize OP-Modified Peptide Conjugates Corresponding to AChE Initially Modified by VX, Sarin, Soman and Tabun and Their Aged Products

Polyclonal antibodies are prepared against 4-VX, 4-sarin, 4-soman, 4-tabun (which corresponds to the initially formed adduct of between AChE and the reactive organophosphoryl) and 5a (which corresponds to aged AChE which forms subsequent to initial exposure of AChE to VX, sarin and soman) and 5b and 5b′ which correspond to the two, aged conjugates following initial tabun exposure. The six antibodies are prepared along with antibodies for 3a and 3b (phospho-decapeptide) referred to as anti-AChE_{10S} and anti-AChE_{10Sp}, respectively, according to the procedure given in Example 3, permits the detection of soman, VX, and tabun-modified AChE (initially inhibited and aged forms) while controlling for unmodified AChE and for phosphorylation by non-specific phosphorylation by inorganic agents.

Antigens for eliciting the antibodies are prepared using the following steps.

(1) Attachment of the 4-VX, 4-soman, 4-tabun, and aged (3b) decapeptides to linker groups.

(2) Conjugation of the linker-decapeptides to immunogenic proteins (KLH, etc.)—analysis of the conjugate to hapten ratio.

(3) Decapeptide-linker-KLH conjugates injected into rabbits to generate the corresponding polyclonal antibodies. Determine cross-reactivity, specificity and epitope map for each.

Antibodies are evaluated for titers, cross-reactivity, selectivity, and stability.

### Example 9

### Preparation of Monoclonal Antibodies Specific for Native, Phosphopeptide and the OP-Modified Peptide Conjugates

Antigens for eliciting the antibodies are prepared using the steps described in Example 9

Monoclonal antibodies are produced according to Table 2. Hybridoma development has four phases: immunization, fusion, cloning and hybridoma stabilization and summarized in the Table 2. Typically, five female Balb/c mice are immunized with the immunogen emulsified with adjuvant. Subsequent injections follow a three-week cycle in which samples are drawn ten days after each injection. The animals' responses to immunogen are assessed by ELISA

For fusion, spleen cells from hyper-immunized mice are prepared and fused to P3X63Ag8.653 or SP2/OAG14 myelomas. Viable hybridomas are selected and screened for antigen specific antibodies by ELISA. The antibody secreting hybridomas (up to 48) with the highest titer are grown and media from positive hybridomas are screened and preliminarily epitope mapped. Positive, primary clones are expanded and selected for re-cloning. Wells with growing cells are screened for antibody secretion by ELISA and evaluated. Each single clone is re-cloned to generate stable, third generation cell lines. The growing cells are screened for antigen specific antibody by ELISA. Cell lines are selected for final expansion for long term storage or scale up by in vitro methods or ascites production.

TABLE 2
MOUSE IMMUNIZATION
PHASE PROTOCOL

| Day | Procedure |
|---|---|
| 0 | Balb/C Mouse - Female |
| | Pre-bleed (~0.1 ml |
| | serum/mouse) |
| | 1° SC: 100 μg with FCA |
| 21 | Boost SC: 100 μg with |
| | FIA |
| 42 | Boost SC: 50 μg with |
| | FIA |
| 52 | Test Bleed (~0.1 ml |
| | serum/mouse) |
| 53 | ELISA Test (1 to 5 |
| | mice) |
| 63 | Boost SC: 50 μg with |
| | FIA |
| 73 | Test Bleed (~0.1 ml |
| | serum/mouse) |
| 74 | ELISA Test (1 to 5 |
| | mice) |
| 84 | Boost SC: 50 μg with |
| | FIA |
| 94 | Test Bleed (~0.1 ml |
| | serum/mouse) |
| 95 | ELISA Assay (1 to 5 |
| | mice) |
| 98 | Boost IV: 10 μg (pre- |
| | fusion 1 mouse only) |
| 99, | Boost IV: 5 μg (pre- |
| 100 | fusion 1 mouse only) |
| 101 | Terminal Bleed (~0.3 ml |
| | serum, pre-fusion |
| | mouse) |
| 110 | Termination/No bleed |
| | (unused mice) |

Immobilization by covalent bonding of polyclonal or monoclonal antibodies to thin film polymers of Example 9 and 10 are conducted in similar fashion to Example 4.

Blood and saliva samples containing various amounts of OP-AChE conjugates are standardized by the bicinchoninic acid method (Smith, 1985) to contain defined quantities of protein. The samples are directly applied to mAb-PDA-biopolymer film and the level of accuracy and sensitivity in the sensor device with standardized samples.

### Example 10

### Construction of a Device for Detecting OP-Modified AChE in Biological Samples

Described is the development and construction of a device with fluorescence film reader and output control electronics for detecting OP-modified AChE in biological samples.

Parameters such as optical absorption and emission characteristics (wavelength, efficiency, polarization, emission distribution, saturation, bleaching, etc.) are determine for specifications of optical and electrical design necessary to obtain a usable signal. Reaction of the polymer to physical stresses such as: temperature, light, humidity, water and chemicals will determine the storage requirements and robustness of the test slides are also determined. Examination evaluation of key factors affecting the generation of false positive and false negative tests is conducted.

Biosensor Film Characterization: A stimulation source emission detector and its optical design will be determined by the stimulation and emission characteristics of the biopolymer material to be used. The source requirements are determined by stimulation efficiency vs. wavelength, with consideration given to any limitations imposed by polarization, saturation and/or bleaching effects. The detector and optical design are determined by emission intensity vs. wavelength and emission distribution. To insure a robust device, the effect of temperature and humidity on the optical properties is evaluated.

Biosensor Film Reader: The system is comprised of a read head (optical detection module), control electronics, user interface module and power supply. The read head is comprised of an optical stimulation source, a sample docking port and an emission detector. The control electronics include drive/interface electronics for the read head and a microprocessor for data analysis and the user interface.

Read Head: The stimulation/emission wavelength is in the range of from about 500-650 nm. Wavelength separation between the stimulation and emission simplifies the optical and electrical design. In one embodiment the emission wavelengths 557 nm and 618 nm and allow the use of silicon-based photodiodes or CMOS image sensors for measuring the fluorescence measurement. These sensors have high sensitivity at these wavelengths and low noise, high gain devices are available. In addition, the peak excitation wavelength of 547 nm allows the use of low cost LEDs or lamps as the source. In one embodiment a hand-pass filter is used to block the excitation source and provide a filtered signal to the detectors with high signal to noise.

Control Electronics—for the electronics will provide drive circuitry for the optical source, low noise detector amplifier and measurement, and support for a simple user interface for stand alone operation. In addition, a computer interface is optionally used to support data collection and system characterization.

### Numbered Embodiments

Several aspects of the invention and related subject matter include the following numbered embodiments. These embodiments are for illustrative purposes and do not limit the scope of the invention.

### Embodiment 1

An optical sensor comprising a biopolymer material and a biomarker receptor for a biomarker wherein the biomarker receptor is immobilized by a receptor immobilization means of attachment to the biopolymer material wherein binding of a biomarker to said receptor produces a detectable change in an optical property of the biopolymer material.

### Embodiment 2

The optical sensor of embodiment 1 wherein the poly-biopolymer material is a di-acetylenic biopolymer material.

### Embodiment 3

The optical sensor of embodiment 1 wherein the optical property is a calorimetric optical property or a fluorescence optical property.

### Embodiment 4

The optical sensor of embodiment 1 wherein the receptor is an antibody or an antibody fragment, wherein the antibody fragment is comprised of an antibody hypervariable domain.

### Embodiment 5

The optical sensor of embodiment 4 wherein the antibody is a polyclonal antibody or a monoclonal antibody.

### Embodiment 6

The optical sensor of embodiment 4 wherein the antibody fragment is obtained by use of an expression vector wherein said expression vector encodes the antibody fragment or is obtained from proteolytic digestion of a polyclonal antibody or a monoclonal antibody.

### Embodiment 7

The optical sensor of embodiment 1 wherein said receptor immobilization attachment means is by covalent bonding of the antibody or antibody fragment to the biopolymer material.

### Embodiment 8

The optical sensor of embodiment 1 wherein said receptor immobilization attachment means is by noncovalent bonding of the antibody or antibody fragment to the biopolymer material.

### Embodiment 9

The optical sensor of embodiment 1 wherein the receptor immobilization attachment means is by a direct covalent attachment obtained from a combination of an amino acid functional group of an amino acid comprising the antibody or antibody fragment and a biopolymer material functional group.

### Embodiment 10

The optical sensor of embodiment 1 wherein the receptor immobilization attachment means is by an indirect covalent attachment obtained from a combination of an amino acid functional group of an amino acid comprising the antibody or antibody fragment, a biopolymer material functional group and an intervening linker precursor.

### Embodiment 11

The optical sensor of embodiment 9 or 10 wherein the amino acid functional group is the ε-amino group of a lysine amino acid.

### Embodiment 12

The optical sensor of embodiment 9 or 10 wherein the amino acid functional group is the amino group of an N-terminal amino acid.

### Embodiment 13

The optical sensor of embodiment 9 or 10 wherein the amino acid functional group is the carboxylic acid group of a C-terminal amino acid.

### Embodiment 14

The optical sensor of embodiment 9 or 10 wherein the amino acid functional group is the sulfhydryl group of a cysteine amino acid.

### Embodiment 15

The optical sensor of embodiment 9 or 10 wherein the amino acid functional group is an aldehyde group of a chemically or enzymatically modified amino acid.

### Embodiment 16

The optical sensor of embodiment 9 wherein the covalent attachment is defined by an entry of Table 1.

### Embodiment 17

The optical sensor of embodiment 10 wherein the covalent attachment is defined by an entry of Table 2.

### Embodiment 18

The optical sensor of embodiment 1 wherein said biomarker is derived from a combination of an organophosphate compound with a serine hydrolase or a cholinesterase.

### Embodiment 19

The optical sensor of embodiment 1 wherein said biomarker is derived from a combination of an organophosphate compound with a serine hydrolase fragment or a cholinesterase fragment wherein the serine hydrolase fragment or the cholinesterase fragment contains the catalytic serine amino acid.

### Embodiment 20

The optical sensor of embodiment 1 wherein said biomarker is a derived from a combination of an organophosphate compound with a peptide of SEQ ID 2.

### Embodiment 21

The optical sensor of embodiment 18, 19 or 20 wherein the organophosphate compound is a pesticide, a reactive organophosphoryl compound, a serine hydrolase inhibitor, a cholinesterase inhibitor, a pesticide metabolite or a pesticide impurity.

### Embodiment 22

The optical sensor of embodiment 21 wherein the organophosphate compound is a pesticide or a reactive organophosphoryl compound.

### Embodiment 23

The optical sensor of embodiment 21 wherein the organophosphate compound is organophosphoryl compound.

### Embodiment 24

The optical sensor of embodiment 21 wherein the organophosphate compound is a serine hydrolase inhibitor or cholinesterase inhibitor.

### Embodiment 25

The optical sensor of embodiment 21 wherein the organophosphate compound is Acephate, Azinphos-methyl, Bensulide, Chlorethoxyfos, Chlorpyrifos, Chlorpyrifos-methyl, Diazinon, Dichlorvos (DDVP), Dicrotophos, Dimethoate, Disulfoton, Ethoprop, Fenamiphos, Fenthion, Malathion, Methamidophos, Methidathion, Methyl parathion, Mevinphos, Naled, Oxydemeton-methyl, Phorate, Phosalone, Phosmet, Phostebupirim, Pirimiphos-methyl, Profenofos, Terbufos, Tetrachlorvinphos, Tribufos, Trichlorfon, or a metabolite or an impurity thereof.

### Embodiment 26

The optical sensor of embodiment 21 wherein the organophosphate compound is sarin, soman, tabun or VX.

### Embodiment 27

An optical sensor of any one of embodiments 1-26 wherein the optical sensor is capable of accepting an incident energy generated from the source of a fluorescent spectrophotometer, a UV-visible spectrophotometer, a fluorescent lamp or a UV-visible lamp and is cabable of permitting detection of a detectable change in an optical property of the biopolymer material produced by interaction of the biopolymer material with the incident energy so accepted.

### Embodiment 28

The optical sensor of embodiment 27 wherein the optical property is a colorimetric optical property or a fluorescence optical property.

### Embodiment 29

The optical sensor of embodiment 28 wherein the incident energy is generated from the source of a fluorescent spectrophotometer.

### Embodiment 30

The optical sensor of embodiment 28 or 29 wherein the optical property is fluorescence emission or fluorescence polarization.

### Embodiment 1A

An optical sensor module comprising the optical sensor of any one of embodiments 1-26.

### Embodiment 2A

An optical sensor module comprising the optical sensor of any one of embodiments 27-30.

### Embodiment 3A

The optical sensor module of embodiment 1A or 2A wherein the poly-di-acetylenic biopolymer material is a Langmuir-Blodgett film.

### Embodiment 4A

The optical sensor module of embodiment 3A wherein the biopolymer material is immobilized by a biopolymer immobilization means of attachment with the side of the poly-di-acetylenic biopolymer material opposed to the receptor to a biopolymer substrate wherein the biopolymer substrate is transparent to a first wavelength in the wavelength range characteristic of the ultraviolet-visible light spectrum.

### Embodiment 5A

The optical sensor module of embodiment 4A wherein the optical sensor module further comprises a cover opposing the biopolymer substrate wherein the cover is transparent to a second wavelength in the wavelength range characteristic of the ultraviolet-visible light spectrum wherein the edges of the cover and the edges of the biopolymer substrate are attached either directly or through an intervening material to provide a water tight seal and a gap between the cover and the optical sensor or the optical sensor film wherein said gap is at least the thickness of the optical sensor or the optical sensor film provided the optical sensor module has at least one opening for transfer of a fluid to and from the surface of the optical sensor film to which the receptors are immobilized.

### Embodiment 6A

The optical sensor module of embodiment 5A wherein the sensor module has an introduction opening and a removal opening wherein the introduction opening permits introduction of said fluid and the removal opening permits removal of said fluid.

### Embodiment 7A

The optical sensor module of embodiment 4A, 5A or 6A wherein the biopolymer substrate is transparent to a first wavelength in the wavelength range characteristic of the ultraviolet-visible light spectrum.

### Embodiment 8A

The optical sensor module of embodiment 4A, 5A or 6A wherein the biopolymer substrate is transparent to a first wavelength in the wavelength range characteristic of the ultraviolet light spectrum.

### Embodiment 9A

The optical sensor module of embodiment 4A, 5A or 6A wherein the biopolymer substrate is transparent to a first wavelength in a first wavelength range of 200-900 nm.

### Embodiment 10A

The optical sensor module of embodiment 4A, 5A or 6A wherein the biopolymer substrate is transparent to a first wavelength of about 220 nm.

### Embodiment 11A

The optical sensor module of embodiment 4A, 5A or 6A wherein the optically transparent biopolymer substrate is transparent to a first wavelength of about 254 nm.

### Embodiment 12A

The optical sensor module of embodiment 4A, 5A or 6A wherein the optically transparent biopolymer substrate is transparent to a first wavelength of about 350 nm.

### Embodiment 13A

The optical sensor module of embodiment 4A, 5A or 6A wherein the cover is transparent to a second wavelength in the wavelength range characteristic of the ultraviolet light spectrum.

### Embodiment 14A

The optical sensor module of embodiment 5A or 6A wherein the cover is transparent to a second wavelength in the wavelength range characteristic of the visible light spectrum.

### Embodiment 15A

The optical sensor module of embodiment 5A or 6A wherein the cover is transparent to a second wavelength in a second wavelength range of 200-900 nm.

### Embodiment 16A

The optical sensor module of embodiment 5A or 6A wherein the second wavelength is about 220 nm.

### Embodiment 17A

The optical sensor module of embodiment 5A or 6A wherein the second wavelength is about 254 nm.

### Embodiment 18A

The optical sensor module of embodiment 5A or 6A wherein the second wavelength is about 350 nm.

### Embodiment 19A

The optical sensor module of embodiment 5A wherein the optical sensor module comprises a cuvette wherein the biopolymer material is immobilized by the biopolymer attachment means to an inside surface of the cuvette.

### Embodiment 20A

The optical sensor module of embodiment 5A wherein the optical sensor module comprises a microtiter plate wherein the biopolymer material is immobilized to the inside bottom surface of one or more wells of the microtiter plate.

### Embodiment 21A

An optical sensor module of any one of embodiments 1A-20A wherein the optical sensor of the optical sensor module is capable of accepting an incident energy generated from the source of a fluorescent spectrophotometer, a UV-visible spectrophotometer, a fluorescent lamp or a UV-visible lamp, and capable of permitting detection of a detectable change in an optical property of the biopolymer material produced by interaction of the biopolymer material with the incident energy so accepted.

### Embodiment 22A

The optical sensor module of embodiment 21A wherein the optical property is a colorimetric optical property or a fluorescence optical property.

### Embodiment 23A

The optical sensor module of embodiment 22A wherein the incident energy is generated from the source of a fluorescent spectrophotometer.

### Embodiment 24A

The optical sensor module of embodiment 22A or 23A wherein the optical property is fluorescence emission or fluorescence polarization.

### Embodiment 1B

An array of optical sensors of any one of embodiments 1-26 wherein the array comprises an ordered arrangement of a plurality of optical sensors; wherein the array is characterized by a plurality of receptors wherein at least two receptors are selective for two different biomarkers wherein each receptor is immobilized by a receptor immobilization means of attachment to the same or different biopolymer material wherein binding of a biomarker to the receptor that is selective for the biomarker produces a detectable change in an optical property of the biopolymer material to which the selective receptor is immobilized.

### Embodiment 2B

The array of embodiment 1B wherein each receptor of the optical sensor array is selective for a different biomarker.

### Embodiment 3B

The array of embodiment 1B or 2B wherein the ordered arrangement of a plurality of optical sensors comprises a first series of optical sensors arranged in parallel rows or columns wherein the receptor of each member of said first series is selective for each member of a second series of biomarkers.

### Embodiment 4B

The array of embodiment 3B wherein the receptor of each member of said first series of receptors is an antibody or an antibody fragment, wherein the antibody fragment is comprised of an antibody hypervariable domain.

### Embodiment 5B

The array of embodiment 4B wherein the receptor is a polyclonal antibody or a monoclonal antibody.

### Embodiment 6B

The array of embodiment 4B wherein the antibody fragment is obtained by a use of an expression vector wherein said expression vector encodes the antibody fragment or is obtained from proteolytic digestion of a polyclonal antibody or a monoclonal antibody.

### Embodiment 7B

The array of embodiment 4B wherein each member of the second series of biomarkers is derived from a combination of a member of a third series of organophosphate compounds with a serine hydrolase or a cholinesterase.

### Embodiment 8B

The array of embodiment 4B wherein each biomarker member of the second series is derived from a reaction product of a different organophosphate member of the third series with a fragment of a serine hydrolase or a cholinesterase wherein the fragment contains the catalytic serine amino acid.

### Embodiment 9B

The array of embodiment 1B or 2B wherein the ordered array is a machine readable or a machine addressable array.

### Embodiment 10B

The array of embodiment 1B or 2B wherein the ordered array is a linear sequence.

### Embodiment 11B

An array of any one of embodiments 1B-10B wherein each optical sensor of the array is capable of accepting an incident energy generated from the source of a fluorescent spectrophotometer, a UV-visible spectrophotometer, a fluorescent lamp or a UV-visible lamp and permitting detection of each detectable change of each optical property in each biopolymer material produced by interaction of each biopolymer material with the incident energy so accepted.

### Embodiment 12B

The array of embodiment 11B and the incident energy is generated from the source of a fluorescent spectrophotometer.

### Embodiment 13B

The optical sensor of embodiment 11B or 12B wherein the optical property is fluorescence emission or fluorescence polarization.

### Embodiment 14B

The array of embodiment 11B wherein the ordered array is a linear sequence and the optical property is a colorimetric optical property.

### Embodiment 15B

The array of embodiment 14B wherein the incident energy is generated from the source of a UV-visible lamp.

### Embodiment 16B

The array of embodiment 14B or 15B wherein at least two of the biopolymer materials have distinct detectable changes in the same optical property wherein the incident energy so accepted is the same.

### Embodiment 17B

The array of embodiment 16B wherein the optical property is emission of visible light.

### Embodiment 18B

The array of embodiment 11B wherein each optical sensor is comprised of the same biopolymer material.

### Embodiment 19B

The array of embodiment 11B wherein the biopolymer material of each optical sensor is physically separate.

### Embodiment 20B

The array of embodiment 11B, 18B or 19B wherein the optical sensor array is a machine readable array or a machine addressable array.

### Embodiment 1C

An optical sensor module which comprises the array of any one of embodiments 1B-20B.

### Embodiment 2C

The optical sensor module of embodiment 1C wherein the poly-di-acetylenic biopolymer material of each optical sensor of the array is a Langmuir-Blodgett film.

### Embodiment 3C

The optical sensor module of embodiment 1C or 2C wherein each biopolymer material is immobilized by a biopolymer immobilization means of attachment with the side of each poly-di-acetylenic biopolymer material opposed to the receptor of each optical sensor to a biopolymer substrate.

### Embodiment 4C

The optical sensor module of embodiment 3C wherein the sensor module further comprises a cover on the side opposing each biopolymer substrate to which each biopolymer material is immobilized wherein the edges of the cover and the edges of the biopolymer substrate are attached either directly or through an intervening material to provide a water tight seal and a gap between the cover and the optical sensor or optical sensor film wherein said gap is at least the thickness of the optical sensor or the optical sensor film provided the optical sensor module has at least one opening for transfer of a fluid to and from the surface of the optical sensor or optical sensor film to which each receptor is immobilized.

### Embodiment 5C

The optical sensor module of embodiment 3C wherein the optical sensor module has an introduction opening and a removal opening wherein the introduction opening permits introduction of said fluid and the removal opening permits removal of said fluid.

### Embodiment 6C

The optical sensor module of embodiment 3C, 4C or 5C wherein the biopolymer substrate is transparent to a first wavelength in the wavelength range characteristic of the ultraviolet-visible light spectrum.

### Embodiment 7C

The optical sensor module of embodiment 3C, 4C or 5C wherein the biopolymer substrate is transparent to a first wavelength in the wavelength range characteristic of the ultraviolet light spectrum.

### Embodiment 8C

The optical sensor module of embodiment 3C, 4C or 5C wherein the biopolymer substrate is transparent to a first wavelength in a first wavelength range of 200-900 nm.

### Embodiment 9C

The optical sensor module of embodiment 3C, 4C or 5C wherein the biopolymer substrate is transparent to a first wavelength of about 220 nm.

### Embodiment 10C

The optical sensor module of embodiment 3C, 4C or 5C wherein the biopolymer substrate is transparent to a first wavelength of about 254 nm.

### Embodiment 11C

The optical sensor module of embodiment 3C, 4C or 5C wherein the biopolymer substrate is transparent to a first wavelength of about 350 nm.

### Embodiment 12C

The optical sensor module of embodiment 4C or 5C wherein the cover is transparent to a second wavelength in the wavelength range characteristic of the ultraviolet-visible light spectrum.

### Embodiment 13C

The optical sensor module of embodiment 4C or 5C wherein the cover is transparent to a second wavelength in the wavelength range characteristic of the visible light spectrum.

### Embodiment 14C

The optical sensor module of embodiment 4C or 5C wherein the cover is transparent to a second wavelength range of 200-900 nm.

### Embodiment 15C

The optical sensor module of embodiment 4C wherein the second wavelength is about 220 nm.

### Embodiment 16C

The optical sensor module of embodiment 4C wherein the second wavelength is about 254 nm.

### Embodiment 17C

The optical sensor module of embodiment 4C wherein the second wavelength is about 350 nm.

### Embodiment 18C

The optical sensor module of embodiment 4C wherein the optical sensor module comprises a microtiter plate wherein each biopolymer material of each optical sensor is immobilized to the inside bottom surface of a different well of the microtiter plate.

### Embodiment 19C

An optical sensor module of any one of embodiments 1C-18C wherein each optical sensor within the array is capable of accepting an incident energy generated from the source of a fluorescent spectrophotometer or a UV-visible spectrophotometer and permitting detection of each detectable change in each optical property in each biopolymer material produced by interaction of each biopolymer material with the incident energy so accepted.

### Embodiment 20C

The array of embodiment 19C and the incident energy is generated from the source of a fluorescent spectrophotometer.

### Embodiment 21C

The array of embodiment 19C or 20C wherein the optical property is a fluorescence optical property.

### Embodiment 22C

The array of embodiment 19C wherein the ordered array is a linear sequence and the optical property is a colorimetric optical property.

### Embodiment 23C

The array of embodiment 22C wherein the incident energy is generated from the source of a UV-visible lamp.

### Embodiment 24C

The array of embodiment 22C or 23C wherein at least two of the biopolymer materials have distinct detectable changes in the same optical property wherein the incident energy so accepted is the same.

### Embodiment 25C

The array of embodiment 24C wherein the optical property is emission of visible light.

### Embodiment 26C

The array of embodiment 19C wherein the biopolymer material of each optical sensor is the same biopolymer material.

### Embodiment 27C

The array of embodiment 19C wherein the biopolymer material of each optical sensor is physically separate.

### Embodiment 28C

The array of embodiment 27C wherein the biopolymer substrate material of each optical sensor is the same biopolymer substrate material and is physically separate.

### Embodiment 29C

The array of embodiment 28C wherein the cover of each optical material is the same cover material and is physically separate.

### Embodiment 30C

The array of embodiment of any one of embodiments 19C, 26C-29C wherein the optical sensor array is a machine readable array or a machine addressable array.

### Embodiment 1D

A kit comprising two or more optical sensors of any one of embodiments 27-30, 11B-20B or optical sensor modules of any one of embodiments 21A-24A, 19C-30C wherein each optical sensor or each optical sensor module has an optimal detectable change in an optical property for a different incident energy.

### Embodiment 2D

An article of manufacture comprising packaging material, an optical sensor, an optical sensor biosensor or a kit of embodiment 1D contained within packaging material, and a label that indicates that the module is for use in a biosensor device.

### Embodiment 1E

A biosensor device comprising the optical sensor module of any one of embodiments 21A-24A, 19C-30C.

### Embodiment 2E

A biosensor device comprising the optical sensor module of any one of embodiments 21A-24A, 19C-30C and a fluorescent spectrophotometer or a UV-visible spectrophotometer adapted for use with the optical sensor module.

### Embodiment 3E

A biosensor device comprising the optical sensor module of any one of embodiments 21A-24A, 19C-30C and a fluorescence detector system adapted for use with the optical sensor module.

### Embodiment 4E

The biosensor device of embodiment 2E or 3E further comprising a liquid handling system or a microfluidic module.

### Embodiment 1F

A method of detecting a biomarker comprising the steps of (a) applying a biological fluid containing said biomarker to an optical sensor of any one of embodiments 27-30, 11B-20B, the optical sensors within an optical sensor module of any one of embodiments 21A-24A, 19C-30C; or to one or more optical sensors within an array of 11B-20B, 19C-30C (b) directing an incident energy to the optical sensor, (c) detecting a detectable change in an optical property in a biopolymer material of at least one optical sensor produced by interaction of accepted incident energy with the biopolymer material.

### Embodiment 2F

The method of embodiment 1F further comprising the step of washing the optical sensor or optical sensor module with a buffer solution.

### Embodiment 3F

The method of embodiment 1F wherein the biological fluid is blood, serum or saliva of a mammal.

### Embodiment 4F

The method of embodiment 3F wherein the mammal has been exposed or is prone to be exposed to an organophosphate compound wherein the organophosphate compound is a pesticide or a reactive organophosphoryl compound.

### Embodiment 5F

The method of embodiment 1F wherein the incident energy has a wavelength in the wavelength range characteristic of the ultraviolet-visible light spectrum.

### Embodiment 6F

The method of embodiment 1F comprising an array of optical sensors within an optical sensor module of embodiments 19C-30C wherein two more of the optical sensors accept an incident of the same or different wavelength either simultaneously or near simultaneously.

Embodiment of 7F: The method of embodiment 1F comprising an array of optical sensors of an optical sensor module of any one of embodiments 19-30C wherein two more of the optical sensors accept an incident of the same or different wavelength in a time resolved sequence.

### Embodiment 8F

The embodiment of 6F or 7F further comprising the step of de-convolution of two or more detectable changes in the same optical property wherein said detectable changes occur simultaneously or near simultaneously.

Variations and modifications of the numbered embodiments, the claims and the remaining portion of the description will be apparent to the skilled artisan after a reading thereof. Such variations and modifications are within the scope and spirit of this invention. All patent citations reported herein are incorporated herein by reference in their entirety.

### CITATIONS

Citations indicated in the specification by author and dates are provided below and are specifically incorporated by reference in their entirity.
Aslam, M. and Dent, A. (1998) Bioconjugation, Macmillan References.
Ausubel et al. Current Protocols in Molecular Biology, for example pages 3.17.1-10).
Benoiton (2006) Chemistry of peptide synthesis CRC Press.
Bloor, D. and R. R. Chance, 1985, Polydiacetylenes: NATO ASI Series E; Applied Science. Martin Nijhoff Publishers, Dordrecht.
Bodansky (1988) Peptide synthesis: A practical textbook, Springer-Verlag.
Bruehl, R. E., F. Dasgupta, T. Katsumoto, J. H. Tan, C. R. Bertozzi, W. Spevak, D. J. Ahn, S. D. Rosen, J. O, Nagy, (2001). Polymerized Liposome Assemblies Bifunctional Macromolecular Selectin Inhibitors Mimicking Physiological Selectin Ligands. Biochem., 40, 5964.
R. R. Chance, G. N. Patel, J. D. Witt (1979), Thermal effects on the optical properties of single crystals and solution-cast films of urethane substituted polydiacetylenes. J. Chem. Phys. 71: 206
Charych, D. H., J. O. Nagy, W. Spevak, M. D. Bednarski (1993). Direct colorimetric detection of a receptor-ligand interaction by a polymerized bilayer assembly. Science, 261, 585.
Charych, D. H., Q. Cheng, A. Reichert, G. Kuziemko, M. Stroh, J. O. Nagy, W. Spevak, R. C. Stevens (1996). A ‘Litmus Test’ for Molecular Recognition Using Artificial Membranes. Chem. and Biol. 3, 113.
Coligan et al. (2000) “Native chemical ligation of polypeptides” Wiley: 18.4.1-21.
Day, D., H. Ringsdorf, (1978). Polymerization of diacetylene carbonic acid monolayers at the gas-water interface. J. Polym. Sci., Polym. Lett. Ed., 16, 205.
Dawson et al. (1994) Science 266:776-9.
Duncan, R. J. S., Weston, P. D. and Wrigglesworth, R. (1983). A new reagent which may be used to introduce sulfhydryl groups into proteins, and its use in the preparation of conjugates for immunoassay. Anal. Biochem. 132:68-73.
Edward, S. L.; Skerritt, J. H.; Hill, A. S.; McAdam, D. P. (1993) An improved immunoassay for chlorpyriphos methyl in grain. Food and Agricultural Immunology 5, 129-144.
Ellman, G. L., Courtney K. D., Andres V. and Featherstone R. M. (1961) A New and Rapid Colorimetric Determination of Acetylcholinesterase Activity. Biochem Pharmacol, 7, 88-95.
Foord, et al. (2005) “International union of pharmacology XLVI: G protein-coupled receptor list” Pharm. Rev., 57:279:288.
Frinkin, M. et al. (1974) Peptide synthesis, Ann. Rev. Biochem. 43:419-443.
George, K.; Schule, T.; Sandoval, L. E.; Jennings, L.; Taylor, P.; Thompson, C. M. (2003) Differentiation between Acetylcholinesterase and the Organophosphate-inhibited Form Using Antibodies and the Correlation of Antibody Recognition with Reactivation Mechanism and Rate. J. Biol. Chem. 278, 45512-45518.
Geoghean, K. F.; Stroh, J. G. (1992) Bioconjugate Chem., 3:138.
Geppetti, Ed. (1999) “Peptidergic G protein-coupled receptors” NATO Science series. Series A: Life Sciences, Vol. 307, IOS Press,
Gill, I., Ballesteros, A. (2003). Immunoglobulin-Polydiacetylene sol-gel nanocomposites as solid-state chromatic biosensors. Angew. Chem. Int. Ed. 2003, 42, 3264-3267.
Golding, Monoclonal Antibodies: Principles and Practice, 2 ed., (1986) Academic Press.
Goldman, L. R. and Koduru S. (2000). Chemicals in the environment and developmental toxicity to children: a public health and policy perspective. Environ Health Perspect, 108 Suppl 3:443-8 (see also: “http://www.epa.gov/pesticides/cumulative/rra-op/”)
Greene (1999), “Protective groups in organic synthesis, 3^{rd} ed.” Wiley Interscience.
Han, et al. (2004) Recent development of peptide coupling agents in organic synthesis, Tet. 60:2447-2476.
Harlow & Lane (1988), Antibodies: A Laboratory Manual, Cold Spring Harbor Laboratory.
Huston, et al. (1988) Proc. Nat. Acad. Sci. USA, 85: 5879-5883.
John, A. E., Lukacs, N. W., Berlin, A. A., Palecanda, A., Bargatze, R. F., Stoolman, L. M., Nagy, J. O. Polymerized lipid nanoparticles-novel P-selectin inhibitors with anti-inflammatory effects in allergic airway disease, FASEB J. 2003; 17:2296.
Joost (2002) Genome Biol., 3(11):research0063.1-0063.16.
Jones, W. T.; Harvey, D.; Jones, S. D; Ryan, G. B.; Wynberg, H.; Ten Hoeve, W.; Reynolds, P. H. S. (1995) Monoclonal antibodies specific for the organophosphate pesticide azinphos-methyl. Food and Agric. Immunol. 7, 9-19.
Jones et al., (1986) Nature 321, 522-525.
Horwell (1995) Trends Biotechnol. 13(4): 132-134
Li L, Wartchow C A, Danthi S N, Shen Z, Dechene N, Pease J, Choi H S, Doede T, Chu P, Ning S, Lee D Y, Bednarski M D, Knox S J. (2004). A novel antiangiogenesis therapy using an integrin antagonist or anti-Flk-1 antibody coated 90Y-labeled nanoparticles. Int J Radiat Oncol Biol Phys. 2004 58:1215-27.
Lio, A. A. Reichert, D. J. Ahn, J. O, Nagy, M. Salmeron, D. H. Charych, “Molecular Imaging of Thermochromic Carbohydrate-Modified Polydiacetylene Thin Films”, Langmuir, 1997, 13, 6524-6532.
Lucas, A. D; Gee, S. J; Hammock, B. D; Seiber, J. N (1995). Integration of immunochemical methods with other analytical techniques for pesticide residue determination. Journal of AOAC International. 78: 585-591.
Maniatis et al. (1982). Molecular Cloning: A Laboratory Manual, Cold Spring Harbor Laboratory
McAdam, D. P.; Hill, A. S.; Beasley, H. L; Skerritt, J. H. (1992). Mono- and polyclonal antibodies to the organophosphate fenitrothion. I. ApproAChEs to hapten-protein conjugation. J. Agric. Food Chem. 40: 1466-1470.
McAdam, D. P.; Skerritt, J. H. (1993). Synthesis of organothiophosphate antigens for the development of specific immunoassays. Aust. J. Chem. 46: 959-967.
Mirzayanov (1995) “Dismantling the Soviet/Russian Chemical Weapons Complex An Insider's View” Chemical Weapons Disarmament in Russia: Problems and Prospects (Washington, D.C.: Henry L. Stimson Center)
Moronne, M. M., Nagy, J. O., Charych, D. H., (2003) U.S. patent application Ser. No. 10/215,736.
Morrison et al., (1984) Proc. Nat. Acad. Sci. USA 81, 6851-6855,
W. Muller, H. Ringsdorf, E. Rump, G. Wildburg, X. Zhang, L. Angermaier, W. Knoll, M. Liley, J. Spinke (1993). Attempts to mimic docking processes of the immune system: recognition-induced formation of protein multilayers. Science, 262: 1706.
Mullis et al. (1987) Methods Enzymol. 155:335-50
R. A. Nallicheri, M. F. Rubner, (1991), Macromolecules, 24, 517.
New (1989) Liposomes: A Practical Approach, IRL Press, Oxford
O'Shannessy, D. J. and Quarles, R. H. (1985). Specific conjugation reactions of the oligosaccharide moieties of immunoglobulins. J. Applied Biochem. 7:347-355.
Paquette, Leo A.; “Principles of Modern Heterocyclic Chemistry” (W. A. Benjamin, New York, 1968), particularly Chapters 1, 3, 4, 6, 7, and 9;
Reichert. A, J. O. Nagy, W. Spevak, D. Charych, (1995), J. Am. Chem. Soc., 117, 829.
Rosenstock, L.; Keifer, M.; Daniell, W. E.; McConnell, R.; Claypoole, K. et. al. (1991) Chronic nervous system effects of acute organophosphate pesticide intoxication. The Lancet 338: 223-227.
Rosoff (1996) Vesicles, Marcel Dekker, Inc., New York
Sambrook et al. (1989) “Molecular Cloning: A Laboratory Manual,” 2nd ed.
Schlessinger (2006) “Epitome: database of structure inferred antigenic epitopes” Nucl. Acid Res., 34:D777-7890.
Skerrift, J. H; Lee, N. (1996) ApproAChEs to the synthesis of haptens for immunoassay of organophosphate and synthetic pyrethroid insecticides. Immunoassays for residue analysis:food safety. ACS, Washington, D.C., 124-149.
Simon et al. (1992) PNAS 89(20): 9367-9371.
Smith, P. K.; Krohn, R. I.; Hermanson, G. T.; Mallia, A. K.; Gartner, F. H.; Provenzano, M. D.; Fujimoto, E. K.; Goeke, N. M.; Olson, B. J.; Klenk, D. C. (1985) Measurement of protein using bicinchoninic acid, Anal. Biochem. 150, 76-85.
Spaulding, R. S.; George, K. M.; Thompson, C. M (2006) Analysis and Sequencing of the Active Site of Acetylcholinesterase by Electrospray Ionization-Quadrupole Time-of-Flight (QTOF) Mass Spectrometry. J. Chromatography B., 830, 105-113.
Spevak, W., J. O, Nagy, D. H. Charych, M. E. Schaefer, J. H. Gilbert, M. D. Bednarski. (1993) Polymerized Liposomes Containing C-Glycosides of Sialic Acid are Potent Inhibitors of Influenza Virus in vitro Infectivity. J. Amer. Chem. Soc., 115:1146.
Spevak W, Foxall C, Charych D H, Dasgupta F, Nagy J. O. (1996). Carbohydrates in an acidic multivalent assembly: nanomolar P-selectin inhibitors. J. Med. Chem. 39(5):1018-20.
W. Spevak, J. O, Nagy, D. H. Charych (1995). Molecular assemblies of functionalized polydiacetylenes. Advanced Materials, 7, 85.
Watson, S; Arkinstall, S. (1994) “The G-Protein Linked Receptor Facts Book”, Academic Press.
Tieke, B. G. Lieser (1982). Influences of the structure of long-chain diynoic acids on their polymerization properties in Langmuir-Blodgett multilayers. J. Colloid Interface Sci., 88, 471.
Thomas, B. N., C. R. Safinya, R. J. Piano, N. A. Clark, 1995, Science, 267, 1635.
Thomson A. Ed. (1994) “The Cytokine Handbook” 2^{nd} ed., Academic Press.
Ulman (1991) An Introduction to Ultrathin Organic Films: From Langmuir-Blodgett to Self-Assembly, Academic Press, Inc., Boston.
Gaines (1966) Insoluble Monolayers at Liquid-Gas Interfaces, Interscience Publishers, New York
Van Emon, J. M.; Mumma, R. O. (1990). Immunochemical Methods of Analysis. ACS Symposium Series 442; ACS, Washington, D.C.
Woodgett, Ed (1994) Nuclear Receptors Nomenclature Committee “A unified nomenclature system for the nuclear receptor superfamily” Cell 1999, 97 (2):161-3. in “Protein Kinases”, IRL Press.
Wong (1993) “Chemistry of protein conjugation and cross-linking”, CRC Press.
Zhang, Z., et al (2004) “Genomic analysis of the nuclear receptor family: New insights into structure, regulation, and evolution from the rat genome”. Genome Res, 14 (4):580-90.
“The Chemistry of Heterocyclic Compounds, A series of Monographs” (John Wiley & Sons, New York, 1950 to present), in particular Volumes 13, 14, 16, 19, and 28.
Definitive rules for nomenclature of organic chemistry J. Amer. Chem. Soc. 1960, 82:5566-5573

What is claimed is:

1. An optical sensor comprising a biopolymer material and a plurality of biomarker receptors wherein each biomarker receptor is covalently attached, optionally through a linker, to a biopolymer material wherein the biomarker receptor is an antibody or a fragment thereof capable of binding a biomarker, wherein binding of the biomarker to said biomarker receptor induces a detectable change in an optical property of the biopolymer material and wherein the biopolymer material is a poly-di-acetylene biopolymer and the biomarker is comprised of a polypeptide and a phosphorous containing moiety wherein the phosphorous containing moiety is derived from an organophosphate compound or a metabolite thereof.

2. The optical sensor of claim 1 wherein the structure of the optical sensor is represented by the formula BMR-L-BIOM wherein BMR is the biomarker receptor, L is the linker and BIOM is the poly-di-acetylene biopolymer.

3. (canceled)

4. The optical sensor of claim 2 wherein the phosphorous containing moiety is covalently bonded a serine oxygen atom of the polypeptide.

5. The optical sensor of claim 2 wherein the organophosphate compound is an organophosphoryl pesticide or a metabolite thereof.

6. The optical sensor of claim 2 wherein the organophosphate compound has structure XYP(O)Z, XYP(S)Z, 1a or 1b 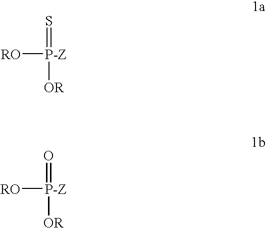
wherein R is methyl or ethyl, X and Y independently are alkoxy, alkylthioester or amine and Z is phenoxy, substituted phenoxy or other good leaving group.

7. The optical sensor of claim 2 wherein the polypeptide is comprised of the polypeptide of SEQ ID 2.

8. The optical sensor of claim 2 wherein the polypeptide is a choline esterase or a fragment thereof.

9. The optical sensor of claim 8 wherein the cholinesterase is a mammalian acetylcholinesterase.

10. The optical sensor of claim 8 wherein the organophosphate compound is an organophosphoryl pesticide of Table 1 or a metabolite thereof.

11. The optical sensor of claim 1 wherein the antibody is a polyclonal or monoclonal antibody.

12. The optical sensor of claim 11 wherein the poly-di-acetylene biopolymer is a film or liposome.

13. The optical sensor of claim 12 wherein the organophosphate compound is an organophosphoryl pesticide.

14. The optical sensor of claim 13 wherein the detectable change in optical property of the biopolymer material is fluorescence emission.

15. An optical sensor module comprising a biopolymer support and an optical sensor of claim 1 immobilized to the support by non-covalent binding.

16. The optical sensor module of claim 15 wherein the biopolymer support is a hydrophobized glass support or one or more wells of a microtiter plate wherein the biopolymer is non-covalently attached to the microtiter plate wells or the glass support.

17. An article of manufacture comprising packaging material, an optical sensor module adapted for use in a biosensor device, and a label that indicates the sensor module is for use in the biosensor device.

18. A machine addressable array of optical sensor modules comprising one or more optical sensors of claim 2 and one or more wells of a microtiter plate into which are immobilized the optical sensors.

19. A method of detecting a biomarker comprising the steps of
(a) applying a biological fluid containing or suspected of containing a biomarker to an optical sensor of claim 1;
(b) directing incident light having a wavelength in the uv-vis spectrum to the biopolymer material of the optical sensor;
(c) determining a change in an optical property of the biopolymer material.

20. A biosensor device comprising an optical sensor module of claim 15.

21. The biosensor device of claim 20 further comprising a fluorescence detector system.

22. The biosensor device of claim 21 further comprising a liquid handling system.

23. The optical sensor of claim 12 wherein the organophosphate compound has the structure XYP(O)Z wherein X is —CH₃, Y is —OiPr, —OEt or —CH(Me)(t-Bu) and Z is —F, —CN or —SCH₂CH₂N(iPr)₂.


SEQUENCE LISTING (13 sequences)

SEQ ID NO: 1 - protein, 5 aa, Artificial
  Synthetic peptide
TSXTS

SEQ ID NO: 2 - protein, 10 aa, Artificial
  Synthetic peptide
TLFGESAGAA

SEQ ID NO: 3 - protein, 12 aa, Artificial
  Synthetic peptide
VTLFGESAGAAS

SEQ ID NO: 4 - protein, 11 aa, Artificial
  Synthetic peptide
TLFGESAGAAS

SEQ ID NO: 5 - protein, 10 aa, Artificial
  Synthetic peptide
TLFGESAGAA

SEQ ID NO: 6 - protein, 10 aa, Artificial
  Synthetic peptide
LFGESAGAAS

SEQ ID NO: 7 - protein, 10 aa, Artificial
  Synthetic peptide
VTLFGESAGA

SEQ ID NO: 8 - protein, 12 aa, Artificial
  Synthetic peptide
VTIFGESAGGES

SEQ ID NO: 9 - protein, 10 aa, Artificial
  Synthetic peptide
TIFGESAGGE

SEQ ID NO: 10 - protein, 11 aa, Artificial
  Synthetic peptide
TIFGESAGGES

SEQ ID NO: 11 - protein, 11 aa, Artificial
  Synthetic peptide
VTIFGESAGGE

SEQ ID NO: 12 - protein, 10 aa, Artificial
  Synthetic peptide
IFGESAGGES

SEQ ID NO: 13 - protein, 10 aa, Artificial
  Synthetic peptide
VTIFGESAGG